(19) United States
(12) Patent Application Publication
(10) Pub. No.: US 2026/0263312 A1
(43) Pub. Date: Sep. 10, 2026

(54) VASOCOMPRESSION DEVICES AND METHODS OF DELIVERING VASOCOMPRESSION THERAPY TO A PATIENT UNDERGOING CHEMOTHERAPY TREATMENT

(71) Applicant: Guangzhou Follisave Technology Development Ltd., Guangzhou (CN)

(72) Inventors: Amir BELSON, Savyon (IL); Yuval ZIPORY, Modiin (IL)

(21) Appl. No.: 19/165,545

(22) PCT Filed: Mar. 15, 2024
(86) PCT No.: PCT/IB2024/000103

§ 371 Date: Sep. 15, 2025

Related U.S. Application Data
(60) Provisional application No. 63/490,487, filed on Mar. 15, 2023.

(51) Int. Cl. A61H 9/00

(52) U.S. Cl. CPC A61H 9/0092; A61H 2201/1607; A61H 2205/02

(57) ABSTRACT

Various devices and methods useful for providing vasocompression to a patient who is undergoing chemotherapy treatment are described. Some devices include a helmet with an inflatable bladder, a facepiece coupled to the helmet and one or more face tiles within the facepiece. Additionally, there may also be structures capable of providing a vasocompression counter force to increase the comfort of a patient who is receiving vasocompression therapy while undergoing a chemotherapy treatment protocol. During use, a patient may perform one or more of altering the pressure of an inflatable bladder, adjusting of one or more straps connected to a facepiece or altering a position or property of one or more face tiles.

### CROSS REFERENCE TO RELATED APPLICATIONS

This application claims priority to U.S. Provisional Patent Application No. 63/490,487 entitled “VASOCOMPRESSION DEVICES AND METHODS OF DELIVERING VASOCOMPRESSION THERAPY TO A PATIENT UNDERGOING CHEMOTHERAPY TREATMENT,” filed Mar. 15, 2023, which is incorporated by reference in its entirety herein.

### INCORPORATION BY REFERENCE

All publications and patent applications mentioned in this specification are herein incorporated by reference to the same extent as if each individual publication or patent application was specifically and individually indicated to be incorporated by reference.

### FIELD

The various embodiments to which this application relates are directed to devices and methods to mitigate or eliminate chemotherapy induced alopecia (CIA), chemotherapy drug-induced hair loss, hair follicle death and/or anagen effluvium. Additionally or optionally, the devices and methods may be used in conjunction with a medication which aids in these objectives.

### BACKGROUND

Chemotherapeutic agents induce CIA by interfering with the transition between the stages of the hair follicle development, stimulating follicular dystrophy or the induction of premature follicle regression {Paus et al. 1994; Trueb, 2009; Yeager et al. 2011}. Telogen effluvium occurs when a larger proportion of hairs in anagen progress prematurely into the telogen phase; for example, cyclophosphamide causes CIA by this mechanism {Patel et al. 2014}. Anagen effluvium, is the most common kind of CIA, because at any given time up to 90% of scalp hair is in anagen and occurs within days to a few weeks after the administration of cytotoxic agents {Olsen et al. 2011}; it is stimulated by alkylating agents, antimetabolites, vinca alkaloids, topoisomerase inhibitors, anthracyclines and taxanes {Espinosa et al. 2003; Yun et al. 2007}. The hair follicle is particularly sensitive to chemotherapy drugs because, as mentioned above, up to 90% of them are in anagen {Batchelor et al. 2001} with their associated matrix keratinocytes representing the most rapidly dividing cell subset, thus they are especially targeted by cell replication-targeting agents.

Currently, scalp cooling represents the only available approach to prevent chemotherapy induced alopecia and may interfere with CIA via a variety of possible mechanisms. Firstly cooling causes blood vessel vasoconstriction, which has been shown to reduce blood flow in the scalp to 20-40% of the normal rate (Janssen et al. 2007). It has been suggested that this will result in less chemotherapeutic drug being delivered to the hair follicles (Bulow et al. 1985). Another possibility is that the rate of drug diffusion across a plasma membrane may be reduced by cooling and thus lower effective drug doses may enter the cells (Lane et al. 1987). Moreover, as cell division is metabolism-driven, it is possible that this process could be decelerated by cooling as temperature can particularly affect phases such as GI and S (Watanabe and Okada. 1967), which could be especially important for drugs that target specific phases of the cell cycle, such as microtubule-destructive drugs targeting mitosis. Also a decrease in the metabolic activity of the cells in the hair follicle could cause a more general reduction in the cytotoxicity of chemotherapy drugs (Bülow et al. 1985). In practice, it is likely that a combination of these methods has a role in the success of scalp cooling in reducing CIA.

In any event scalp cooling does not work in all cases and efficacy varies from person to person.

As a result, improvements are still needed in the techniques used to mitigate, substantially eliminate or aid in the treatment of chemotherapy drug-induced hair loss, hair follicle death and/or anagen effluvium.

### SUMMARY OF THE DISCLOSURE

In general, in one embodiment, a head covering for inducing vasocompression in the blood supply of a patient receiving chemotherapy includes a shell having a front edge, a back edge, a top, a left side edge and a right side edge, a liner within the shell shaped to at least partially accommodate the hair of the patient, at least one vasocompression element within the outer shell, and a head covering retention system coupled to the shell.

This and other embodiments can include one or more of the following features. The shell retention system can include a submandibular strap connected to the shell. The shell retention system can include one or more of a rear strap, a front strap or a neck strap connected to the shell. The shell retention system can include one or more adjustment elements. The at least one vasocompression element can be an expandable bladder within the liner. The at least one vasocompression element can be coupled to the shell. The head covering can further include a pressure source in communication with the expandable bladder. The head covering can further include a continuous perimeter connecting the front edge, the back edge, the left side edge and the right-side edge. The head covering of the at least one vasocompression element can further include an expandable feature responsive to a pressure source to expand away from the shell. The head covering can further include an expandable layer coupled to the liner. The at least one vasocompression element can be on the expandable layer. The head covering of the at least one vasocompression element can further include one or more raised features. The head covering of the liner within the shell shaped to at least partially accommodate the hair of the patient can further include one or more features on the liner to engage with the hair of the patient. The liner or the at least one vasocompression element can be smooth. The liner or the at least one vasocompression element can include an array of evenly distributed protrusions having straight or circular inflatable structures. The head covering retention system can further include a hook and loop fastener, a buckle, a quick release mechanism or an adjustment feature. The head covering can further include a gas source in communication with the liner or the at least one vasocompression element for inflation of a portion of the liner or a portion of the at least one vasocompression element. The head covering can further include at least one relief valve to prevent over pressurization of an inflatable element of the head covering. The gas source can be a manual pump mechanism, a bulb, an electric pump, or pressurized tank, cartridge or reservoir. The head covering can further include a control system for adjusting the flow of gas from the gas source to maintain a desired pressure within the head covering. The desired pressure within the head covering can be sufficient to produce a vasocompression result while minimizing discomfort or side effects to the patient or harm to surrounding structures. The head covering can further include a cooling gas source in communication with the liner or the at least one vasocompression element wherein a portion of the liner or a portion of the at least one vasocompression element is adapted to provide cooling to an interior portion of the head covering. The head covering can further include one or more pressure sensors within an interior portion of the head covering. The one or more pressure sensors can provide an indication of pressure to the control system. The one or more pressure sensors can be adapted to indicate an amount of pressure applied to a patient undergoing chemotherapy when the patient is wearing the head covering. The head covering can further include one or more sensors to measure skin perfusion (O₂ sat) or Doppler blood flow monitor or infra-red pulse oximeter sensor. Sensors may also be used to detect that blood flow in the vasculature receiving vasocompression therapy is stopped or slowed sufficiently to prevent exposure of hair follicles to the chemotherapeutic agent. The head covering can further include a continuous structure from the occiput area to the eyebrows or to the forehead. The head covering can further include a structure or an inflatable structure to compress the eyebrows for a vasocompression effect on the eyebrows. The head covering can further include a timer and a control system adapted to provide an automatic deflation protocol when the timer has elapsed or to cease operation of any active vasocompression element. The head covering can further include a timer and a control system adapted for programmed automatic inflation and deflation of the liner or the one or more vasocompression elements or the activation and deactivation of the one or more vasocompression elements. The liner can be a rigid or semi-elastic layer configured for an exact fit for the patient to fit exactly the shape of the patient's head and hair. The head covering retaining system can include a tightening system. The head covering can further include one or more selective pressure points positioned to compress one or more vessels of the cranial circulatory system of a patient. The head covering can further include a vibrating element coupled to the liner or the at least one vasocompression element. The liner and the at least one vasocompression elements can be sized, shaped, or positioned relative to the head covering based on the hair style of the patient.

In general, in one embodiment, a head covering for inducing vasocompression in the blood supply of a patient receiving chemotherapy includes a cap having a front edge, a back edge, a top, a left side edge and a right-side edge, a liner within the cap shaped to at least partially accommodate the hair of the patient, and at least one vasocompression element within the cap.

This and other embodiments can include one or more of the following features. The cap can include a flexible structure, a partially flexible structure, a combination of semi-rigid structure and flexible structure or an adjustable structure sized to remain in place on the head when the at least on vasocompression element is active. The at least one vasocompression element can be an expandable bladder within the cap or the liner. The at least one vasocompression element can be coupled to the cap or the liner. The head covering can further include a pressure source in communication with the expandable bladder or the at least one vasocompression element. The head covering can further include a continuous perimeter connecting the front edge, the back edge, the left side edge and the right side edge. The head covering of the at least one vasocompression element can further include an expandable feature responsive to a pressure source to expand away from the cap. The head covering can further include an expandable layer coupled to the liner wherein the at least one vasocompression element can be on the expandable layer. The head covering of the at least one vasocompression element can further include one or more raised features. The head covering of the liner within the shell shaped to at least partially accommodate the hair of the patient can further include one or more features on the liner to engage with the hair of the patient. The liner or the at least one vasocompression element can be smooth. The liner or the at least one vasocompression element can include an array of evenly distributed protrusions having straight or circular inflatable structures. The head covering can further include a gas source in communication with the liner or the at least one vasocompression element for inflation of a portion of the liner or a portion of the at least one vasocompression element. The head covering can further include at least one relief valve to prevent over pressurization of an inflatable element of the head covering. The gas source can be a manual pump mechanism, a bulb, an electric pump, or pressurized tank, cartridge or reservoir. The head covering can further include a control system for adjusting the flow of gas from the gas source to maintain a desired pressure within the head covering. The desired pressure within the head covering can be sufficient to produce a vasocompression result while minimizing discomfort or side effects to the patient or harm to surrounding structures. The head covering can further include a cooling gas source in communication with the liner or the at least one vasocompression element wherein a portion of the liner or a portion of the at least one vasocompression element is adapted to provide cooling to an interior portion of the head covering. The head covering can further include one or more pressure sensors within an interior portion of the head covering. The one or more pressure sensors can provide an indication of pressure to the control system. The one or more pressure sensors can be adapted to indicate an amount of pressure applied to a patient undergoing chemotherapy when the patient is wearing the head covering. The head covering can further include one or more sensors to measure skin perfusion (O₂ sat) or Doppler blood flow monitor or infra-red pulse oximeter sensor. The head covering can further include a continuous structure from the occiput area to the eyebrows or to the forehead. The head covering can further include a structure or an inflatable structure to compress the eyebrows for a vasocompression effect on the eyebrows. The head covering can further include a timer and a control system adapted to provide an automatic deflation protocol when the timer has elapsed or to cease operation of any active vasocompression element. The head covering can further include a timer and a control system adapted for programmed automatic inflation and deflation of the liner or the activation and deactivation of the one or more vasocompression elements. The liner can be a rigid or semi-elastic layer configured for an exact fit for the patient to fit exactly the shape of the patient's head and hair. The head covering retaining system can include a tightening system. The head covering can further include one or more selective pressure points positioned to compress one or more vessels of the cranial circulatory system of a patient. The head covering can further include a vibrating element coupled to the liner or the at least one vasocompression element. The liner and the at least one vasocompression elements can be sized, shaped, or positioned relative to the head covering based on the hair style of the patient.

In general, in one embodiment, a method of inducing vasocompression in a cranial circulatory system of a patient receiving chemotherapy includes: (1) placing a head covering about the head of the patient; (2) at least partially inducing vasocompression in a portion of the cranial circulatory system of the patient using a vasocompression element of the head covering; (3) Initiating a chemotherapy session with the patient; (4) concluding the chemotherapy session with the patient; and (5) ceasing the step of at least partially inducing vasocompression in a portion of the cranial circulatory system of the patient when a period of time has elapsed after the step of concluding the chemotherapy session sufficient to partially mitigate an effect of chemotherapy induced alopecia in the patient.

This and other embodiments can include one or more of the following features. The method can further include engaging a head covering retention system to immobilize the head covering relative to the head of the patient. The period of time that has elapsed after the step of concluding the chemotherapy session can be up to 24 hours. The period of time that has elapsed after the step of concluding the chemotherapy session can be at least one hour. The period of time that has elapsed after the step of concluding the chemotherapy session can be at least 48 hours. The vasocompression element of the head covering can be a liner wherein a step of expanding the liner or a step of contracting the liner is performed during the step of at least partially inducing vasocompression in a portion of the cranial circulatory system of the patient. There can be an increase in the amount of vasocompression during the step of expanding the liner corresponding to an increase of the force of the liner against the scalp of the patient. There can be a decrease in the amount of vasoconstriction during the step of contracting the liner corresponding to a decrease of the force of the liner against the scalp of the patient. The vasocompression element of the head covering can be a liner having one or more features wherein a step of expanding the liner or a step of contracting the liner is performed during the step of at least partially inducing vasocompression in a portion of the cranial circulatory system of the patient. There can be an increase in the amount of vasocompression during the step of expanding the liner corresponding to an increase of the force of the one or more features on the liner against the scalp of the patient. There can be a decrease in the amount of vasocompression during the step of contracting the liner corresponding to a decrease of the force of the one or more features on the liner against the scalp of the patient. The vasocompression element of the head covering can be an inflatable bladder wherein the degree of vasocompression during the step of at least partially inducing vasocompression in a portion of the cranial circulatory system of the patient corresponds at least in part to the amount of pressure within the inflatable bladder. The vasocompression element of the head covering can be an inflatable bladder wherein the degree of vasocompression during the step of at least partially inducing vasocompression in a portion of the cranial circulatory system of the patient is increased or decreased by raising or lowering the pressure in the inflatable bladder against the skull. The vasocompression element of the head covering can be one or more features arranged on an interior surface of the head covering which are urged into contact with a portion of the patient's scalp having hair follicles during the step of at least partially inducing vasocompression in a portion of the cranial circulatory system of the patient. The vasocompression element of the head covering can be one or more features arranged on an interior surface of the head covering which correspond to an arrangement of the patient's hair, wherein the one of more features act on the arrangement of the patient's hair to further the performance of the step of least partially inducing vasocompression in a portion of the cranial circulatory system of the patient. During the at least partially inducing vasocompression step, the vasocompression element of the head covering can compress a portion of the scalp containing a plurality of hair follicles against a portion of the cranium to at least partially vasocompress the blood supply to the plurality of hair follicles. The portion of the cranium can be one or more of a superior temporal line, an inferior temporal line, a parietal bone, a squamous suture, a temporal bone, a lambdoid suture, an occipital bone, a mastoid process, a sphenoid, a portion in proximity to the supraorbital foramen, a frontal bone, a coronal suture. During the at least partially inducing vasocompression step, the vasocompression element of the head covering can be positioned to at least partially vasocompress an artery or a vein of the cranial circulatory system of the patient by compressing the artery or the vein against a portion of the cranium. The artery or the vein of the cranial circulatory system can be a branch of an external carotid artery or a branch of the internal carotid artery. The artery or the vein of the cranial circulatory system can be an ophthalmic artery. The artery or the vein of the cranial circulatory system can be a superficial temporal artery. The artery or the vein of the cranial circulatory system can be a posterior auricular artery. The artery or the vein of the cranial circulatory system can be an occipital artery. The artery or the vein of the cranial circulatory system can be a supraorbital artery or a supratrochlear artery. The portion of the cranium can be one or more of a superior temporal line, an inferior temporal line, a parietal bone, a squamous suture, a temporal bone, a lambdoid suture, an occipital bone, a mastoid process, a sphenoid, a portion in proximity to the supraorbital foramen, a frontal bone, a coronal suture. The step of at least partially inducing vasocompression in a portion of the cranial circulatory system can result from compression of a portion of the scalp containing a blood vessel of the cranial circulatory system of the patient against a portion of the periosteum of the patient. The step of at least partially inducing vasocompression in a portion of the cranial circulatory system can be produced by interaction between the vasocompression element of the head covering and a blood vessel of the cranial circulatory system of the patient. The method of the step of at least partially inducing vasocompression in a portion of the cranial circulatory system can further include compressing a portion of the scalp against a portion of the periosteum to induce vasocompression in a portion of a vascular bed of a hair follicle. The vasocompression element of the head covering can be positioned with respect to the patient's head wherein during the engaging step the vasocompression element is positioned in proximity a specific vein or a specific artery of the cranial circulatory system of the patient to at least partially vasocompress the specific vein or the specific artery. The specific vein or the specific artery can be any of the blood vessels mentioned above. The method can further include delivery of a vasoconstrictor medication to the patient before, during or after use of any methods or devices described herein. The vasoconstrictor medication can be one of an alpha-adrenoceptor agonist, a vasopressin analog, an epinephrine, a norepinephrine, a phenylephrine, a dopamine, a dobutamine, a migraine medication, a headache medication, a serotonin 5-hydroxytryptamine agonist, and a triptans. The method or device as in any of the above, wherein the patient can be receiving chemotherapy by an injection, an infusion, a targeted therapy, a hormone therapy or an immunotherapy. The patient can be receiving chemotherapy by an intravenous method, an intrathecal method, an intra-arterial method, an intracavitary method, an intramuscular method, an intralesional method, or an intravesical method. The patient can be receiving chemotherapy in a home, a health care provider office, a clinic, an outpatient infusion center, a hospital infusion center, or a hospital in-patient room.

In general, in one embodiment, a head covering for inducing vasocompression in the blood supply of a patient receiving chemotherapy includes a shell having a front edge, a back edge, a top, a left side edge and a right side edge, a liner within the shell shaped to at least partially accommodate the hair of the patient, at least one vasocompression element within the shell or the liner and a head covering retention system including a full-face support structure that encircles the mandible body and Ramus, extends across and at least partially covering the Zygomatic, Maxilla and Frontal bones of the skull of the patient with an opening in the full-face support structure for a pair of eyes, a nose and a mouth of the patient, a cushion along an inner wall of the full-face support between an inner wall of the full-face support and the patient; and one or more straps between the head covering and the full-face support.

This and other embodiments can include one or more of the following features. The head covering of the cushion can further include a plurality of tile face cushions arranged along the inner wall of the full-face support wherein the size, shape, firmness, compression modulus characteristics of each tile face cushion of the plurality of tile face cushions may be selected based on a location within the full-face support and a loading characteristic of the location while vasocompression therapy is being delivered.

The head covering can further include a plurality of tile face cushions arranged along the inner wall of the full-face support, wherein each tile face cushion of the plurality of tile face cushions can include a post; and a plurality of apertures within the full-face support wherein the location of each aperture can be adapted and configured to correspond to a post of a tile face cushion proximate to the aperture.

The one or more straps can further include at least two front support straps extending from the front edge of the shell to a top edge of the full-face support, or at least two right side support straps extending from a right side of the full-face support structure to right side of the shell, or at least two left side support straps extending from a left side of the full-face support structure to left side of the shell; or any of a combination of the above.

The liner can be an inflatable liner.

The inflatable liner can have an inner zone against the liner and an outer zone to engage the patient.

A separation zone can be provided between the inner zone and the outer zone. The separation zone can form a perimeter that corresponds to the perimeter of the shell. The separation zone can retain its shape during inflation and deflation of the inflatable liner. Each of the plurality of tile face cushions can include an inner surface contoured to a portion of a face and an outer surface contoured to engage with a portion of the full-face support and a mid-portion between the inner surface and the outer surface.

The mid-portion of each face tile can be one or a combination of a gel, a foam, a silicon, a uniform durometer materials, a mixed durometer material or an inflation zone. Each face tile can have a post for alignment of each face piece tile to a corresponding opening in the full face covering.

Each face tile can have a shaft and a knob for mechanical adjustment of the spacing of the inner surface of the face tile from the inner surface of the full face covering or a valve for adjusting a pressure within a face tile.

In general, in one embodiment, a head covering for inducing vasocompression in the blood supply of a patient receiving chemotherapy includes a shell having a front edge, a back edge, a top, a left side edge and a right side edge, an inflatable liner within the shell, a head covering retention system adapted to maintain the position of the shell and the inflatable liner relative to a head of the patient, a face covering coupled to the shell, and at least one pad support within the face covering.

This and other embodiments can include one or more of the following features. The face covering can include a plurality of connection sites providing access for a plurality of support pads within the face covering.

The at least one pad support can be a plurality of individual pads adapted and configured to at least partially conform to a bony structure of the patient.

The plurality of individual pads can be controllable in applying pressure to a portion of the patient's face using a mechanical adjustment system.

The plurality of individual pads can be controllable in applying pressure to a portion of the patient's face using an electrotechnical adjustment system.

The electrotechnical adjustment system can further include a time based or used selected program of increasing or decreasing the pressure applied by a pad to a portion of the patient's face.

The user selected program can be controlled using a control interface, remote device, smart device or app selected by the user based on pre-set pressure settings, prior chemotherapy sessions or based on time-based adjustments to relieve intermittent fatigue while using the vasocompression device.

The head covering can further include a vasocompression counterforce structure can be a ring having an inner diameter from 1 inch to 4 inches.

The vasocompression counterforce structure can have a frustoconical shape with a base having an outer diameter of 6 to 8 inches and an upper surface having a ring with a diameter ranging from 1 inch to 4 inches.

The vasocompression counterforce structure can have a base, a stem and an upper support surface. A diameter of the base can be from 1-3 inches. The vasocompression counterforce structure can have a base, an elongated stem and a rectangular upper support surface.

The vasocompression counterforce structure can have a frustoconical shape with a base having an outer diameter of 6 to 8 inches and an upper ring supporting a base, an elongated stem and an upper support surface.

In general, in one embodiment, a head covering for inducing vasocompression in the blood supply of a patient receiving chemotherapy includes a shell having a front edge, a back edge, a top, a left side edge and a right side edge, at least one vasocompression element within the shell, a head covering retention system adapted to maintain the position of the shell and liner relative to a head of the patient, and a vasocompression counterforce structure coupled to the shell.

This and other embodiments can include one or more of the following features. The vasocompression counterforce structure can be a ring having an inner diameter from 1 inch to 4 inches.

The vasocompression counterforce structure can have a frustoconical shape with a base having an outer diameter of 6 to 8 inches and an upper surface having a ring with a diameter ranging from 1 inch to 4 inches.

The vasocompression counterforce structure can have a base, a stem and an upper support surface, wherein a diameter of the base is from 1-3 inches.

The vasocompression counterforce structure can have a base, an elongated stem and a rectangular upper support surface.

The vasocompression counterforce structure can have a frustoconical shape with a base having an outer diameter of 6 to 8 inches and an upper ring supporting a base, an elongated stem and an upper support surface.

In general, in one embodiment, a head covering for inducing vasocompression in the blood supply of a patient receiving chemotherapy includes a shell having a front edge, a back edge, a top, a left side edge and a right side edge, a liner within the shell shaped to at least partially accommodate the hair of the patient, at least one vasocompression element within the shell, a head covering retention system including a submandibular rigid support extending from a left side of a mandible body around the mental protuberance to a right side of a mandible body, a cushion on submandibular rigid support, a front left support strap extending from the front edge of the shell to the submandibular rigid support, a front right support strap extending from the front edge of the shell to the submandibular rigid support a left-side support strap extending from the left-side edge of the shell to the left end of the submandibular rigid support, and a right-side support strap extending from the right-side edge of the shell to the right end of the submandibular rigid support.

This and other embodiments can include one or more of the following features. The cushion can extend above the submandibular rigid support and beyond the left and right ends of the submandibular rigid support.

The cushion can be a foam filler within the structure of the submandibular rigid support.

The submandibular rigid support can extend from a position proximate to a left side angle of the mandible, along the left side of a mandible body around the mental protuberance, along the right side of a mandible body to a position proximate to a right-side angle of the mandible.

The head covering can further include a vasocompression counterforce structure with a dynamic facepiece.

The head covering can further include a vasocompression counterforce structure or a dynamic facepiece.

The head covering retention system can include one or more of a rear strap, a front strap or a neck strap connected to the shell.

The shell retention system can include one or more adjustment elements.

The at least one vasocompression element can be an expandable bladder within the liner.

The at least one vasocompression element can be coupled to the shell.

The head covering can further include a pressure source in communication with the expandable bladder.

The head covering can further include a continuous perimeter connecting the front edge, the back edge, the left side edge and the right side edge.

The head covering of the at least one vasocompression element can further include an expandable feature responsive to a pressure source to expand away from the shell.

The head covering can further include an expandable layer coupled to the liner wherein the at least one vasocompression element is on the expandable layer.

The head covering of the at least one vasocompression element can further include one or more raised features.

The liner within the shell can be shaped to at least partially accommodate the hair of the patient and can further include one or more features on the liner to engage with the hair of the patient.

The liner or the at least one vasocompression element can be smooth.

The liner or the at least one vasocompression element can include an array of evenly distributed protrusions having straight or circular inflatable structures.

The head covering retention system can further include a hook and loop fastener, a buckle, a quick release mechanism or an adjustment feature.

The head covering can further include a gas source in communication with the liner or the at least one vasocompression element for inflation of a portion of the liner or a portion of the at least one vasocompression element.

The head covering can further include at least one relief valve to prevent over pressurization of an inflatable element of the head covering.

The gas source can be a manual pump mechanism, a bulb, an electric pump, or pressurized tank, cartridge or reservoir.

The head covering can further include a control system for adjusting the flow of gas from the gas source to maintain a desired pressure within the head covering.

The desired pressure within the head covering can be sufficient to produce a vasocompression result while minimizing discomfort or side effects to the patient or harm to surrounding structures.

The head covering can further include a cooling gas source in communication with the liner or the at least one vasocompression element wherein a portion of the liner or a portion of the at least one vasocompression element can be adapted to provide cooling to an interior portion of the head covering.

The head covering can further include one or more pressure sensors within an interior portion of the head covering.

The one or more pressure sensors can provide an indication of pressure to the control system.

The one or more pressure sensors can be adapted to indicate an amount of pressure applied to a patient undergoing chemotherapy when the patient is wearing the head covering.

The head covering can further include one or more sensors to measure skin perfusion (O₂ sat) or Doppler blood flow monitor or infra-red pulse oximeter sensor or for confirmation that blood flow in vessels undergoing vasocompression has stopped or slowed sufficiently.

The head covering can further include a continuous structure from the occiput area to the eyebrows or to the forehead.

The head covering can further include a structure or an inflatable structure to compress the eyebrows for a vasocompression effect on the eyebrows.

The head covering can further include a timer and a control system adapted to provide an automatic deflation protocol when the timer has elapsed or to cease operation of any active vasocompression element.

The head covering can further include a timer and a control system adapted for programmed automatic inflation and deflation of the liner or the one or more vasocompression elements or the activation and deactivation of the one or more vasocompression elements.

The liner can be a rigid or semi-elastic layer configured for an exact fit for the patient to fit exactly the shape of the patient's head and hair.

The head covering retaining system can further include a tightening system.

The head covering can further include one or more selective pressure points positioned to compress one or more vessels of the cranial circulatory system of a patient.

The head covering can further include a vibrating element coupled to the liner or the at least one vasocompression element.

The liner and the at least one vasocompression elements can be sized, shaped, or positioned relative to the head covering based on the hair style of the patient.

In general, in one embodiment, a head covering for inducing vasocompression in the blood supply of a patient receiving chemotherapy includes a cap having a front edge, a back edge, a top, a left side edge and a right side edge, a liner within the cap shaped to at least partially accommodate the hair of the patient, and at least one vasocompression element within the cap.

This and other embodiments can include one or more of the following features. The liner can further include a first layer adjacent to the inner surface of the cap and a second layer adapted and configured to engage with the patient's head and a separation zone separating the first layer from the second layer wherein the separation zone has a perimeter that corresponds to the edges of the cap.

The cap can include a flexible structure, a partially flexible structure, a combination of semi-rigid structure and flexible structure or an adjustable structure sized to remain in place on the head when the at least one vasocompression element is active.

The at least one vasocompression element can be an expandable bladder within the cap or the liner.

The at least one vasocompression element can be coupled to the cap or the liner.

The head covering can further include a pressure source in communication with the expandable bladder or the at least one vasocompression element.

The head covering can further include a continuous perimeter connecting the front edge, the back edge, the left side edge and the right side edge.

The at least one vasocompression element can include an expandable feature responsive to a pressure source to expand away from the cap.

The head covering can further include an expandable layer coupled to the liner wherein the at least one vasocompression element can be on the expandable layer.

The at least one vasocompression element can further include one or more raised features.

The liner within the shell shaped to at least partially accommodate the hair of the patient can further include one or more features on the liner to engage with the hair of the patient.

The liner or the at least one vasocompression element can be smooth.

The liner or the at least one vasocompression element can include an array of evenly distributed protrusions having straight or circular inflatable structures.

The head covering can further include a gas source in communication with the liner or the at least one vasocompression element for inflation of a portion of the liner or a portion of the at least one vasocompression element.

The head covering can further include at least one relief valve to prevent over pressurization of an inflatable element of the head covering.

The gas source can be a manual pump mechanism, a bulb, an electric pump, or pressurized tank, cartridge or reservoir.

The head covering can further include a control system for adjusting the flow of gas from the gas source to maintain a desired pressure within the head covering.

The desired pressure within the head covering can be sufficient to produce a vasocompression result while minimizing discomfort or side effects to the patient or harm to surrounding structures.

The head covering can further include a cooling gas source in communication with the liner or the at least one vasocompression element. A portion of the liner or a portion of the at least one vasocompression element can be adapted to provide cooling to an interior portion of the head covering.

The head covering can further include one or more pressure sensors within an interior portion of the head covering.

The one or more pressure sensors can provide an indication of pressure to the control system.

The one or more pressure sensors can be adapted to indicate an amount of pressure applied to a patient undergoing chemotherapy when the patient is wearing the head covering.

The head covering can further include one or more sensors to measure skin perfusion (O₂ sat) or Doppler blood flow monitor or infra-red pulse oximeter sensor.

The head covering can further include a continuous structure from the occiput area to the eyebrows or to the forehead.

The head covering can further include a structure or an inflatable structure to compress the eyebrows for a vasocompression effect on the eyebrows. The head covering can further include a timer and a control system adapted to provide an automatic deflation protocol when the timer has elapsed or to cease operation of any active vasocompression element.

The head covering can further include a timer and a control system adapted for programmed automatic inflation and deflation of the liner or the activation and deactivation of the one or more vasocompression elements.

The liner can be a rigid or semi-elastic layer configured for an exact fit for the patient to fit exactly the shape of the patient's head and hair.

The head covering retaining system can include a tightening system.

The head covering can further include one or more selective pressure points positioned to compress one or more vessels of the cranial circulatory system of a patient.

The head covering can further include a vibrating element coupled to the liner or the at least one vasocompression element.

In general, in one embodiment, a method of inducing vasocompression in a cranial circulatory system of a patient receiving chemotherapy includes: (1) Placing a head covering about the head of the patient; (2) At least partially inducing vasocompression in a portion of the cranial circulatory system of the patient using a vasocompression element of the head covering; (3) Inducing a counter force applied to the head covering sized and directed to offset at least some of the forces introduced to the head of the patient as a result of the at least partially inducing vasocompression step; (4) Initiating a chemotherapy session with the patient; (5) Concluding the chemotherapy session with the patient; and (6) Ceasing the step of at least partially inducing vasocompression in a portion of the cranial circulatory system of the patient when a period of time has elapsed after the step of concluding the chemotherapy session sufficient to partially mitigate an effect of chemotherapy induced alopecia in the patient.

This and other embodiments can include one or more of the following features. The step of inducing a counter force applied to the head covering can be performed by the force acting directly on the shell of the head covering.

The step of inducing a counter force applied to the head covering can be performed by the force acting on a vasocompression counterforce structure coupled to the shell of the head covering.

The method can further include engaging a head covering retention system to immobilize the head covering relative to the head of the patient.

The period of time that has elapsed after the step of concluding the chemotherapy session can be up to 24 hours.

The period of time that has elapsed after the step of concluding the chemotherapy session can be at least one hour.

The period of time that has elapsed after the step of concluding the chemotherapy session can be at least 48 hours.

The vasocompression element of the head covering can be a liner wherein a step of expanding the liner or a step of contracting the liner is performed during the step of at least partially inducing vasocompression in a portion of the cranial circulatory system of the patient.

There can be an increase in the amount of vasocompression during the step of expanding the liner corresponding to an increase of the force of the liner against the scalp of the patient.

There can be a decrease in the amount of vasoconstriction during the step of contracting the liner corresponding to a decrease of the force of the liner against the scalp of the patient.

The vasocompression element of the head covering can be a liner having one or more features wherein a step of expanding the liner or a step of contracting the liner can be performed during the step of at least partially inducing vasocompression in a portion of the cranial circulatory system of the patient.

There can be an increase in the amount of vasocompression during the step of expanding the liner corresponding to an increase of the force of the one or more features on the liner against the scalp of the patient.

There can be a decrease in the amount of vasocompression during the step of contracting the liner corresponding to a decrease of the force of the one or more features on the liner against the scalp of the patient.

The vasocompression element of the head covering can be an inflatable bladder wherein the degree of vasocompression during the step of at least partially inducing vasocompression in a portion of the cranial circulatory system of the patient corresponds at least in part to the amount of pressure within the inflatable bladder.

The vasocompression element of the head covering can be an inflatable bladder wherein the degree of vasocompression during the step of at least partially inducing vasocompression in a portion of the cranial circulatory system of the patient can be increased or decreased by raising or lowering the pressure in the inflatable bladder against the skull.

The vasocompression element of the head covering can be one or more features arranged on an interior surface of the head covering which are urged into contact with a portion of the patient's scalp having hair follicles during the step of at least partially inducing vasocompression in a portion of the cranial circulatory system of the patient.

The vasocompression element of the head covering can be one or more features arranged on an interior surface of the head covering which correspond to an arrangement of the patient's hair, wherein the one of more features act on the arrangement of the patient's hair to further the performance of the step of least partially inducing vasocompression in a portion of the cranial circulatory system of the patient.

During the at least partially inducing vasocompression step, the vasocompression element of the head covering can compress a portion of the scalp containing a plurality of hair follicles against a portion of the cranium to at least partially vasocompress the blood supply to the plurality of hair follicles.

The portion of the cranium can be one or more of a superior temporal line, an inferior temporal line, a parietal bone, a squamous suture, a temporal bone, a lambdoid suture, an occipital bone, a mastoid process, a sphenoid, a portion in proximity to the supraorbital foramen, a frontal bone, a coronal suture.

During the at least partially inducing vasocompression step, the vasocompression element of the head covering can be positioned to at least partially vasocompress an artery or a vein of the cranial circulatory system of the patient by compressing the artery or the vein against a portion of the cranium.

The artery or the vein of the cranial circulatory system can be a branch of an external carotid artery or a branch of the internal carotid artery.

The artery or the vein of the cranial circulatory system can be an ophthalmic artery.

The artery or the vein of the cranial circulatory system can be a superficial temporal artery.

The artery or the vein of the cranial circulatory system can be a posterior auricular artery.

The artery or the vein of the cranial circulatory system can be an occipital artery.

The artery or the vein of the cranial circulatory system can be a supraorbital artery or a supratrochlear artery.

The portion of the cranium can be one or more of a superior temporal line, an inferior temporal line, a parietal bone, a squamous suture, a temporal bone, a lambdoid suture, an occipital bone, a mastoid process, a sphenoid, a portion in proximity to the supraorbital foramen, a frontal bone, a coronal suture.

The step of at least partially inducing vasocompression in a portion of the cranial circulatory system can result from compression of a portion of the scalp containing a blood vessel of the cranial circulatory system of the patient against a portion of the periosteum of the patient.

The step of at least partially inducing vasocompression in a portion of the cranial circulatory system can be produced by interaction between the vasocompression element of the head covering and a blood vessel of the cranial circulatory system of the patient.

The method of the step of at least partially inducing vasocompression in a portion of the cranial circulatory system can include compressing a portion of the scalp against a portion of the periosteum to induce vasocompression in a portion of a vascular bed of a hair follicle.

The vasocompression element of the head covering can be positioned with respect to the patient's head wherein during the engaging step the vasocompression element can be positioned in proximity a specific vein or a specific artery of the cranial circulatory system of the patient to at least partially vasocompress the specific vein or the specific artery.

The specific vein or the specific artery can be any of the blood vessels.

The method can further include delivery of a vasoconstrictor medication to the patient before, during or after any step.

The vasoconstrictor medication can be one of an alpha-adrenoceptor agonist, a vasopressin analog, an epinephrine, a norepinephrine, a phenylephrine, a dopamine, a dobutamine, a migraine medication, a headache medication, a serotonin 5-hydroxytryptamine agonist, and a triptans.

The patient can be receiving chemotherapy by an injection, an infusion, a targeted therapy, a hormone therapy or an immunotherapy.

The patient can be receiving chemotherapy by an intravenous method, an intrathecal method, an intra-arterial method, an intracavitary method, an intramuscular method, an intralesional method, or an intravesical method.

The patient can be receiving chemotherapy in a home, a health care provider office, a clinic, an outpatient infusion center, a hospital infusion center, or a hospital in-patient room.

In general in one embodiment, a method for comfort wear of headgear to protect against hair loss during chemotherapy includes: (1) placing a helmet with a facepiece attached to the helmet via a plurality of straps over a head and face of a user; (2) adjusting the plurality of straps to make contact between a plurality of tiles on the facepiece and the face of the user; (3) inflating a balloon liner of the helmet; (4) adjusting one or more of: (i) a pressure from the contact between the plurality of tiles and the face of the user, (ii) a pressure from the inflated balloon liner of the helmet on the head of the user, and (iii) the plurality of straps, until vasocompression of a blood supply to hair follicles of the user is achieved; (5) initiating a chemotherapy session with the user; and (6) continuing to adjust for comfort of the user during the chemotherapy session, one or more of: the pressure from the contact between the plurality of tiles on the face and the user, the pressure from the inflated balloon liner of the helmet on the head of the user, and the plurality of straps.

This and other embodiments can include one or more of the following features. The plurality of tiles can have an outer surface facing an interior of the facepiece and an inner surface facing the face of the user.

The balloon liner can be inflated via an air adaptor coupled to an inflation line and inflation source.

Adjusting the pressure from the contact between the plurality of tiles on the facepiece can include engaging a knob having a plurality of ridges each having a smooth edge and a jagged edge, wherein the smooth edge can be configured to indicate a first direction of rotation to increase the pressure of the contact between the plurality of tiles and the face of the user by moving one or more of the plurality of tiles towards the face of the user, wherein the jagged edge can be configured to indicate a second direction of rotation to decrease the pressure of the contact between the plurality of tiles and the face of the user by moving one or more of the plurality of tiles away from the face of the user.

The knob can interface with padding and a support piece to increase or decrease one or more of: (i) a distance between the padding/support piece and the face of a patient, and (ii) a pressure on the face of the patient, wherein the padding can be a low durometer silicon pad having 5-20 units Shore A.

The inflatable ballon liner can have a first zone configured to interface with the head of the patient, a second zone configured to interface with an interior surface of the helmet and a boundary layer between the first zone and the second zone, wherein the second zone and the boundary layer can each have a higher durometer than a durometer of the first zone of the balloon liner.

The first zone can have a thickness of 10-100 microns, wherein the second zone can have a thickness of 50-300 microns.

The boundary layer can be of a consistent width in a range of 20-40 mm around a perimeter of the helmet between the first zone and the second zone.

One or more of the facepiece and a position of the plurality of tiles can be pre-contoured to a facial topography of the user prior to putting the facepiece on the face of the user.

The method can further include a waiting period to confirm vasocompression of the blood supply to the hair follicles of the user before initiating the chemotherapy session with the user.

The method can further include offsetting load and pressure on the head or face of the user by resting one or more of the helmet and facepiece being worn by the user against a support surface to generate a desired counter force vector.

The method can further include adjusting one or more of: the pressure from the contact between the plurality of tiles on the face and the user, the pressure from the inflated balloon liner of the helmet (inflation state) on the head of the user, and the plurality of straps before or during the chemotherapy session manually or automatically via a graphical user interface (GUI) of a computer implemented application or a web application, wherein the GUI can adjust one or more of the plurality of tiles, one or more of the plurality of straps, and an inflation state of the balloon liner.

The GUI can adjust the one or more of the plurality of tiles, one or more of the plurality of straps, and the inflation state of the balloon liner via interfacing with a combination chemotherapy and vasocompression therapy control and delivery system via sensors and valves on the helmet, balloon liner, facepiece, plurality of tiles, and pressure sources, further wherein the combination chemotherapy and vasocompression therapy control and delivery system can be responsive to user prescription and treatment preferences, chemotherapy session history, and electronic medical records (EMR) of the user.

In general, in one embodiment, a method for comfort wear of headgear to protect a user against hair loss during chemotherapy includes: (1) Placing a headband about the head of the user; (2) placing a helmet with a facepiece and headband attached to the helmet via a plurality of straps over a head and face of the user so that the headband is between the helmet and the facepiece; (3) adjusting the plurality of straps to make contact between one or more of: (i) a plurality of tiles on the facepiece and the face of the user, and (ii) the headband and a circumference of the head of the user; (4) inflating a balloon liner of the helmet; (5) adjusting one or more of: (i) a pressure from the contact between the plurality of tiles and the face of the user, (ii) a pressure from the inflated balloon liner of the helmet on the head of the user, and (iii) the plurality of straps, until vasocompression of a blood supply to hair follicles of the user is achieved; (6) initiating a chemotherapy session with the user; and (7) continuing to adjust for comfort of the user during the chemotherapy session, one or more of: the pressure from the contact between the plurality of tiles and the face of the user, the pressure from the inflated balloon liner of the helmet on the head of the user, and the plurality of straps.

This and other embodiments can include one or more of the following features. The plurality of straps can include one or more of straps, bands, and adhesive, wherein the headband is configured to perform one or more of: (i) stabilizing the helmet during inflation and deflation of the balloon liner, (ii) offsetting a load on the helmet, and (iii) further restricting circulation of blood to hair follicles of the patient.

The headband can be separately put on the forehead of the user and positioned to be located at a gap between the helmet and the facepiece.

In general, in one embodiment, a device for protecting against hair loss during chemotherapy includes a helmet having an inflatable balloon liner and a plurality of helmet connection points, a facepiece having a plurality of facepiece connection points at a perimeter of the facepiece, and a plurality of straps to connect the facepiece to the helmet via the plurality of helmet connection points and facepiece connection points.

This and other embodiments can include one or more of the following features. The device can further include a headband coupled to the helmet.

The device can further include the inflatable ballon liner having: a first zone that can be configured to interface with the head of the patient, and a second zone that can be configured to interface with an inner surface of the helmet, wherein the first zone can have a lower durometer than a durometer of the second zone of the balloon liner and a boundary zone between the first zone and the second zone wherein the boundary zone can follow a perimeter of the helmet.

The facepiece can have a plurality of tiles, wherein the tiles can be comprised of materials including one or more of foam, foam composite, gel, air, wherein one or more of the tiles can be configured to be adjusted via mechanical or pneumatic mechanisms.

In still other various alternative embodiments, the various method described above may be performed using any of the vasocompression devices described above or elsewhere herein.

In general, in one embodiment, a computer controller can be adapted and configured to control the pressure applied to the scalp and face a patient using any of the above described methods using any one or a combination of the above devices before, during and after a chemotherapy session.

A graphical user interface can be adapted and configured for interaction with the computer controller that can enable selection of one or more of an inflatable element or pressure applying element that may be increased or decreased.

The computer controller or the graphical user interface can further be configured to control, monitor, initiate, suspend or otherwise control delivery a chemotherapy protocol to a patient.

The computer controller or graphical user interface can be adapted and configured for use with a device or implement a method as described above or here in below.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A is a perspective view of a cancer patient at the beginning of a chemotherapy treatment to treat a tumor or malignant lesion in a breast. This view includes a bag containing a chemotherapeutic agent and the amount in the bag at the initiation of the chemotherapeutic treatment session.

FIG. 1B is an enlarged view of the tumor or malignant lesion in the breast in the patient of FIG. A.

FIG. 2A is a view of the bag in FIG. 1A showing the amount delivered and remaining of the chemotherapeutic agent.

FIG. 2B is a graph showing an expected concentration curve of the chemotherapeutic agent in the patient's blood stream as a function of Drug Concentration (mg/L) and Time (min). The triangle marks the estimated concentration based on the amount of chemotherapy agent delivered as indicated by the level of fluid visible in the bag.

FIG. 3A is a view of the patient and the bag in FIG. 1A showing the amount delivered and remaining of the chemotherapeutic agent as indicated by the decreased level of fluid in the bag.

FIG. 3B is the graph of FIG. 2B showing an expected concentration curve of the chemotherapeutic agent in the patient's blood stream as a function of Drug Concentration (mg/L) and Time (min) for the status of the patient in FIG. 3A. The triangle marks the estimated concentration based on the amount of chemotherapy agent delivered as indicated by the decreased level of fluid visible in the bag which is nearly at the peak or maximum drug concentration.

FIG. 4 is a view of the state of a process of chemotherapy-induced necrosis in the targeted tumor or malignant lesion of FIG. 1B.

FIG. 5A is a view of the patient and the bag in FIG. 3A showing the amount delivered and remaining of the chemotherapeutic agent as indicated by the decreased level of fluid in the bag. A Drug Concentration (mg/L) and Time (min) curve for the patient in FIG. 5A would be at, near or beyond the highest level.

FIG. 5B is a view of the advancing state of the process of chemotherapy-induced necrosis in the targeted tumor or malignant lesion of FIGS. 1B and 4.

FIG. 5C is a cross section of the scalp and a hair follicle including the vascular bed before any chemotherapeutic agent reaches the vasculature of the skull, scalp or vascular bed of the hair.

FIG. 5D is a cross section of the scalp and a hair follicle vascular bed of FIG. 5C showing the infiltration and circulation of the chemotherapeutic agent.

FIG. 6A is a view of the final state of chemotherapy-induced necrosis in the targeted tumor or malignant lesion of FIGS. 1B, 4 and 5B.

FIG. 6B is a view of the final state of wide spread chemotherapy-induced necrosis in the follicles of the patient which began in FIG. 5D.

FIG. 6C is a side view of the state of wide spread hair loss in the patient of FIGS. 1A, 3A, and 5A produced by one or a combination of chemotherapy-induced alopecia, hair loss, hair follicle death and/or anagen effluvium.

FIG. 7A is a perspective view of a pulled back portion of the skin and scalp to reveal the layers of tissue over a bony portion of a skull.

FIG. 7B is a cross section of view of the pulled back portion of the skin and scalp of FIG. 7A.

FIG. 7C is a cross section of view of the indicated area of the skin and scalp of FIG. 7B.

FIG. 8 is a cross section of a healthy scalp and a hair follicle including the vascular bed.

FIG. 9 is a perspective view of a head covering for inducing vasocompression in the blood supply of a patient receiving chemotherapy. This view also shows a shell and interior liner which is connected to an external pump or controller for one or more vasocompression elements in the head covering. A submandibular strap connected to the shell is also visible in this view. The head covering is configured to deliver vasocompression to the scalp and eyebrows.

FIG. 10A is a perspective view and partial interior view of a head covering for inducing vasocompression in the blood supply of a patient receiving chemotherapy. This view also shows a shell and interior liner which is connected to a shell mounted pump or controller for one or more vasocompression elements in the head covering. A submandibular strap connected to the shell is also visible in this view. The vasocompression element illustrated is an inwardly expanding manifold where inwardly is toward scalp—away from shell. The head covering is configured to deliver vasocompression to the scalp and eyebrows.

FIG. 10B is a cross section view of a portion of a liner having a vasocompression element with one or more raised features. The raised features have a rounded shape facing the patient who is wearing the head covering.

FIG. 10C is a cross section view of a portion of a liner having a vasocompression element with one or more raised features. The raised features have a rectangular pyramid shape with a flat top section facing the patient who is wearing the head covering.

FIG. 11A is a side view of a patient's head who is wearing a vasocompression element used to deliver vasocompression in the blood supply of the patient while receiving chemotherapy. This view also shows an inflatable liner which is connected to a pump or controller (not visible in this view). The inflatable liner vasocompression element is configured to deliver vasocompression to the scalp and eyebrows.

FIG. 11B is a side view of the patient in FIG. 11A showing the shell and an under the chin strap of the head covering for inducing vasocompression in the blood supply of a patient receiving chemotherapy using the vasocompression element of FIG. 11A to deliver vasocompression to the scalp and eyebrows.

FIG. 12A is a side view of a patient's head who is wearing a head covering including at least one vasocompression element used to deliver vasocompression. This is the state of the patient prior to or shortly after initiation of the patient receiving chemotherapy. A submandibular strap connected to a shell of the head covering is also visible in this view. The head covering is configured to deliver vasocompression to the scalp and eyebrows of the patient.

FIG. 12B is a cross section of a portion of the hair, scalp and hair follicles of the patient in FIG. 12A. This view shows the hair pressed against the scalp when the head covering in donned (FIG. 12A) and prior to the delivery of vasocompression.

FIG. 12C is a cross section of a portion of the hair, scalp and hair follicles of the patient in FIG. 12B. This view shows the expansion of one or more vasocompression elements in the head covering causing vasocompression within the vascular bed of the follicle.

FIG. 12D is an exterior view of the patient undergoing chemotherapy and vasocompression with arrows indicating that vasocompression may be applied to numerous portions of the scalp including the eyebrows.

FIG. 13A is a view of the patient of FIGS. 12A-12D receiving vasocompression from the head covering during a chemotherapy session which is nearly completed as indicated by the nearly empty bag of chemotherapeutic agent.

FIG. 13B is the graph showing an expected concentration curve of the chemotherapeutic agent in the patient's blood stream as a function of Drug Concentration (mg/L) and Time (min) for the status of the patient in FIG. 13A. The triangle marks the estimated concentration based on the amount of chemotherapy agent delivered as indicated by the decreased level of fluid visible in the bag of FIG. 13A. The arrow indicates that the drug concentration amount is nearly at or just beyond the peak or maximum drug concentration.

FIG. 13C is a view of the advancing state of the process of chemotherapy-induced necrosis in the targeted tumor or malignant lesion of the patient of FIGS. 12A, 12D and 13A who has been receiving vasocompression during the delivery of the chemotherapeutic agent.

FIG. 13D is a cross section of the head covering, scalp and a hair follicle of the patient of FIGS. 12A, 12D and 13A who has been receiving vasocompression during the delivery of the chemotherapeutic agent (FIG. 13B). This view shows that the vascular bed is completely free or substantially free of any chemotherapeutic agent or that the level of chemotherapeutic agent that reaches the vascular bed is maintained below a level to harm the follicles.

FIG. 14 is a side view of the patient of FIG. 13A at a time period after the delivery of chemotherapy with vasocompression. The patient has maintained hair on both the head and the eyebrows as a result of the beneficial effects of vasocompression.

FIG. 15 is a flow chart of a representative method for providing vasocompression to a patient undergoing a chemotherapy treatment.

FIG. 16 is a front view of a head covering for inducing vasocompression in the blood supply of a patient receiving chemotherapy. A partial submandibular rigid support extends continuously from a left portion of the mandible body, the mental protuberance or chin, and a right portion of the mandible body. An oversized cushion is shown positioned between the patient's jaw and the rigid support. The rigid support is attached to the shell above the eyebrows and two front attachment points. The rigid support is attached to the shell at the left and right sides from attachment points at the left and right ends of the support.

FIG. 17 is a front view of a head covering for inducing vasocompression in the blood supply of a patient receiving chemotherapy. Similar to FIG. 16, there is a submandibular rigid support attached to the shell. In contrast to FIG. 16, the support extends continuously from a left portion of the mandible body at the left side angle of the mandible, along the mental protuberance or chin, and a right portion of the mandible body to the right-side angle of the mandible. A cushion of similar size to the rigid support is positioned between the patient's jaw and the rigid support. The rigid support is attached to the shell above the eyebrows and two front attachment points adjacent the chin. The rigid support is attached to the shell at the left and right sides from attachment points at the left and right ends of the support.

FIG. 18 is a front view of a head covering for inducing vasocompression in the blood supply of a patient receiving chemotherapy that includes a full-face support structure. The full-face support structure encircles the mandible body and Ramus, extends across and at least partially covering the Zygomatic, Maxilla and Frontal bones of the skull. There is an opening in the full-face support structure for the eyes, nose and mouth. A cushion of similar size and shape to the full-face support structure is positioned between the patient and the full-face support. The full-face support is attached to the shell above the eyes and two top front attachment points. The rigid support is attached to the shell at the left and right sides from attachment points at the left and right ends of the support along the mandible body and a location adjacent to a superior aspect of the Ramus. The full-face support has additional unused attachment points such as in the middle of the forehead, left and right sides adjacent to the nose and left and right sides adjacent to the chin. One or more of the additional attachment points may be used to further adjust the distribution of forces generated during vasocompression.

FIG. 19A is a front view of a head covering for inducing vasocompression in the in the blood supply of a patient receiving chemotherapy that includes a full-face support structure. The full-face support structure encircles the mandible body and Ramus, extends across and at least partially covering the Zygomatic, Maxilla and Frontal bones of the skull. There is an opening in the full-face support structure for the eyes, nose and mouth. A series of openings in the full-face support structure permit the use of an array of independently selectable cushion tiles to be added to the full-face support structure. FIGS. 20 to 25 illustrate various configurations of the cushion tiles adapted for use with the full-face support. The cushion tiles are not shown in the various views of FIGS. 19A, 19B and 19C. The full-face support is attached to the shell at nine points, three each along the forehead, the right side and the left side. Along the forehead is one each above each eye and one in the middle of the forehead above the nose. A pair of upper attachment points are located near the temples. Left and right lower attachment points are located adjacent to the Ramus below the ear. Each of the attachment points are D-shaped connectors sized to receive a loop that is part of a strap attached to an adjustable connection point on the shell. Each one of the straps and associated connectors may be adjusted to fit the patient and while also increasing comfort during use by the distribution of forces generated during vasocompression.

FIG. 19B is a right-side view of a head covering for inducing vasocompression as shown in FIG. 19A. In this view, the top three attachment points along with the pair of right upper attachment points and the right lower attachment point are visible.

FIG. 19C is a left-side view of a head covering for inducing vasocompression as shown in FIG. 19A. In this view, two of the top three attachment points along with the pair of left upper attachment points and the left lower attachment point are visible.

FIG. 19D is a rear view of a head covering for inducing vasocompression as shown in FIG. 19A. In this view, a number of the attachment points on the shell are shown. Additionally, the pneumatic connection and line in the back rear of the shell are visible.

FIG. 20 is a top isometric view of an embodiment of a multiple tile face cushion adapted for use with the embodiments of FIGS. 19A-19D. The full-face shield and the straps are omitted from this view. Each of the tiles has a plug which is sized and positioned to align with and be received by the corresponding opening in the face shield. The cushion properties for each face shield may be adapted by selecting cushion tiles of desired shape, size, composition along with the placement of specific tiles in the location and orientation in the face shield to improve comfort for the patient experiencing vasocompression therapy.

FIG. 21A is a side view of a vasocompression device of FIGS. 19A-19D with the face shield removed showing the position of an exemplary multiple tile face cushion. In this view, the patient is in a prone position with the shell positioned against a support structure which produces a force vector indicated by the arrow. The force vector is used to provide additional comfort to the patient undergoing vasocompression therapy by offsetting a portion of the forces generated during the application of vasocompression therapy. In this embodiment, the counter force is directed from the contact point on the support structure towards the upper attachment point and the nose of the patient.

FIG. 21B is a side view of a vasocompression device of FIG. 21A with the face shield removed showing the position of an exemplary multiple tile face cushion. In this view, the patient is in a prone position with the shell positioned against a support structure which produces a force vector indicated by the arrow. The force vector is used to provide additional comfort to the patient undergoing vasocompression therapy by offsetting a portion of the forces generated during the application of vasocompression therapy. In this embodiment, the counter force is directed from the contact point on the support structure towards the upper attachment point, past the ear and below the chin of the patient.

FIG. 21C is a side view of a vasocompression device of FIG. 21A with the face shield removed showing the position of an exemplary multiple tile face cushion. In this view, the patient is in a prone position with the shell positioned against a support structure which produces a force vector indicated by the arrow. The force vector is used to provide additional comfort to the patient undergoing vasocompression therapy by offsetting a portion of the forces generated during the application of vasocompression therapy. In this embodiment, the counter force is directed from the contact point on the support structure towards the upper attachment point, past the ear and above the chin of the patient.

FIG. 21D is a side view of a vasocompression device of FIG. 21A with the face shield removed showing the position of an exemplary multiple tile face cushion. In this view, the patient is in an upright position and the external force is imparted to the shell which produces a force vector indicated by the arrow. The force vector is used to provide additional comfort to the patient undergoing vasocompression therapy by offsetting a portion of the forces generated during the application of vasocompression therapy. In this embodiment, the counter force is directed from the contact point towards the upper attachment point above the ear and towards a line between the nose and the chin of the patient.

FIG. 22 is a side view of a vasocompression device of FIG. 21A with the face shield removed showing the position of an exemplary multiple tile face cushion. There is a support ring shown in cross section at a point on the shell central to the counter force vector shown in FIG. 21D. In use, the support ring would be placed in contact with a support structure or other fixture used to impart the counter force. The ring would be used to distribute the force across a larger area of the head covering.

FIG. 23 is a rear perspective view of a vasocompression device of FIG. 21A with the face shield removed showing the position of an exemplary multiple tile face cushion. There is a support ring shown in position on the head covering. The support ring has a frustoconical shape with a larger diameter base against the surface of the shell. The flat upper ring surface may be used to receive and distribute a counter force. Optionally, the upper support ring may be used as a support base for other or additional support structures such as in FIG. 26.

FIG. 24A is a side view of a vasocompression device of FIG. 21A with the face shield removed showing the position of an exemplary multiple tile face cushion. There is a support structure shown in position on the head covering. The support structure has a base, a stem and an upper support surface. The base may be sized to distribute the counter force across a larger portion of the shell while the direction and contour of the stem and upper support surface may be configured to engage with a corresponding support surface.

FIG. 24B is a front perspective view of the embodiment of FIG. 24A.

FIG. 25 is a side view of a vasocompression device of FIG. 21A with the face shield removed showing the position of an exemplary multiple tile face cushion.

FIG. 26A is a left side view of an embodiment of a vasocompression device having a stabilization weight and a head support on an adjustable, sliding mount.

FIG. 26B is a front view of the vasocompression device in FIG. 26A.

FIG. 27A is a bottom up perspective view of an interior of a head covering showing the shell and inflatable liner. The air adaptor and connection for adjusting the pressure within the balloon liner is also shown as the rear of the shell.

FIG. 27B is a cross section view of the head covering of FIG. 27A showing the arrangement of the shell, the liner and air adaptor and connections.

FIGS. 28A and 28B are front and rear views of an embodiment of the head covering of FIGS. 27A and 27B shown in position on a head. A series of attachment points are shown extending rearward from the upper corners in the view of FIG. 28A and along the rear surface as shown in FIG. 28B.

FIGS. 29A-29C is an inferior perspective and left perspective view of a balloon liner, respectively.

FIG. 30A is a cross section of an inflatable bladder or balloon.

FIG. 30B is a rear perspective view of the inflatable bladder or balloon of FIG. 30A.

FIG. 31 is a cross section view of an inflatable bladder joint to a helmet with an inflation port.

FIG. 32 is a cross section view of a head covering for inducing vasocompression embodiment similar to FIG. 19C having the tile cushions in place. The detailed section view of the shell shows that there is a secondary layer of material between the outer wall of the inflatable liner and the inner wall of the shell.

FIG. 33A is a left-side perspective view of the balloon liner inside a helmet.

FIGS. 33B-33C is an inferior and left-side perspective view of a balloon liner inside a helmet having a head rest, respectively.

FIG. 34 is a right-side view of an embodiment of the head covering of FIGS. 33A-33C shown in position on a head.

FIG. 35A is a bottom up view of six air cells around the chin.

FIG. 35B is a top down view of the air cells in FIG. 35A.

FIG. 35C is a cross section view of an air cell within support structure in FIG. 35A.

FIG. 35D is a side view of the air cell/pads in FIG. 35A with an exemplary inflation tube connected to one of the air cells/pads.

FIG. 35E is a bottom up view of exemplary size and arrangement of inflated air cells/pads around the chin.

FIG. 36A is a front isometric view of a face shield having eight individual pad adjustment zones with knobs on the face shield in relation to the helmet/shell.

FIG. 36B is a front view of the face shield of FIG. 36A.

FIG. 36C is a front isometric view of only the face shield of FIG. 36A without the helmet.

FIG. 36D is a front view of the face shield of FIG. 36A without the helmet.

FIG. 36E is a rear (interior) view of the face shield of FIG. 36D showing the individual pad shapes and positions each one controllable by operation of the dedicated pad adjustment knob.

FIG. 37A is an isometric view of a helmet with moveable support and dynamic pressure adjustment facepiece.

FIG. 37B is an exploded view of the vasocompression device of FIG. 37A.

FIG. 37C is a front view of the vasocompression device of FIG. 37A.

FIG. 37D is a rear view of the vasocompression device of FIG. 37A.

FIG. 37E is a side view of the vasocompression device of FIG. 37A.

FIG. 38A is a right-side view of a head covering and face support for inducing vasocompression before straps connect the two pieces.

FIG. 38B is a left-side view of a head covering and face support for inducing vasocompression with straps connecting the two pieces and an optional headband.

FIG. 39A is an isometric view of a dynamic facepiece having 18 independently controlled support pads.

FIG. 39B is an interior view of the dynamic facepiece of FIG. 39A.

FIG. 39C is a section view of the dynamic facepiece of FIGS. 39A and 39B with moveable support and various shaped pads.

FIG. 39D is an isometric front view showing an exploded view of a pad adjustment knob in relation to a pad and pad support.

FIG. 39E illustrates an exploded view of a haptic feedback embodiment of a pad adjustment knob.

FIG. 39F is an isometric interior view of the dynamic facepiece of FIG. 39B.

FIG. 40 is a flow chart of a method of delivering chemotherapy having vasocompression and controllable, adjustable pressure relief of a rigid facepiece.

FIG. 41 is a perspective view of a representation of the anatomical landmarks and structure of a mandible.

FIGS. 42A-42B illustrate a left side and front view of the bones of the face and skull, respectively.

FIG. 43 is an exemplary Graphical User Interface (GUI) for controlling pressure in a head covering and face covering.

FIG. 44 is an exemplary Graphical User Interface (GUI) for controlling an individual tile on a face covering.

FIG. 45 is a block diagram of an exemplary computer controller for operating a combination chemotherapy and vasocompression therapy system.

FIG. 46A-46B is a block diagram illustrating a method for comfort wear of headgear for protecting against hair loss during chemotherapy.

FIG. 47A-47B is a block diagram illustrating another method for comfort wear of headgear for protecting against hair loss during chemotherapy.

### DETAILED DESCRIPTION

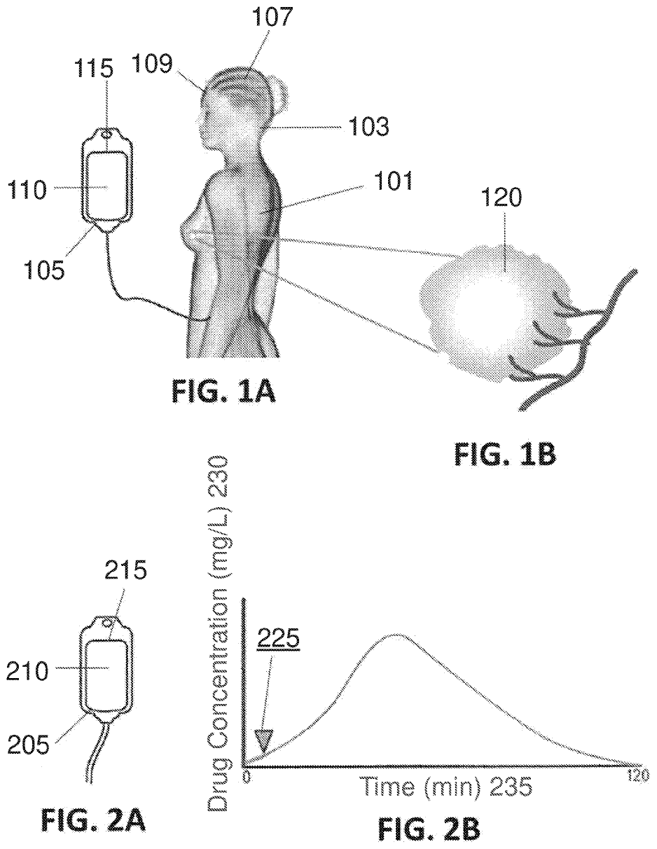

FIG. 1A is a perspective view of a cancer patient 100 at the beginning of a chemotherapy treatment to treat a tumor or malignant lesion 120 in a breast. This view includes an IV bag 105 containing a chemotherapeutic agent 110 and the amount in the bag 115 at the initiation of the chemotherapeutic treatment session. Also shown are the patient's hair 107 and eyebrow region 109. Also shown is the patient's head 103.

FIG. 1B is an enlarged view of the tumor or malignant lesion 120 in the breast in the patient 101 of FIG. 1A.

FIG. 2A is a view of the IV bag 205 corresponding to IV bag 105 from FIG. 1A showing the amount delivered and remaining 215 of the chemotherapeutic agent 210.

FIG. 2B is a graph showing an expected concentration curve of the chemotherapeutic agent 210 in the patient's blood stream as a function of Drug Concentration (mg/L) 230 and Time (min) 235. The triangle 225 marks the estimated concentration based on the amount of chemotherapy agent 210 delivered as indicated by the level of fluid 215 visible in the bag 205.

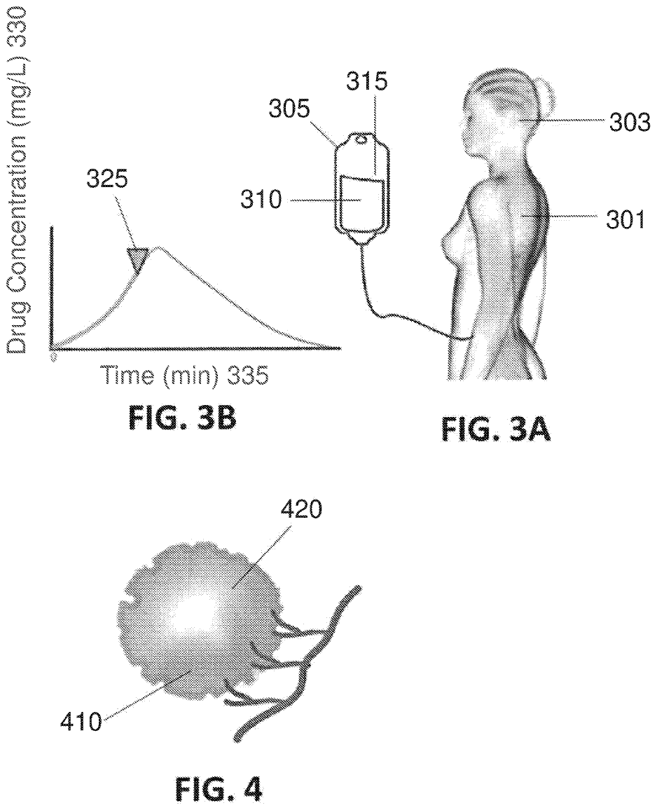

FIG. 3A is a view of the patient and the IV bag 305 in FIG. 1A showing the amount delivered 315 and remaining of the chemotherapeutic agent 310 as indicated by the decreased level of fluid in the bag. Also shown here are the patient's hair follicles 303 and back region 301.

FIG. 3B is the graph of FIG. 2B showing an expected concentration curve of the chemotherapeutic agent 310 in the patient's blood stream as a function of Drug Concentration (mg/L) 330 and Time (min) 335 for the status of the patient in FIG. 3A. The triangle 325 marks the estimated concentration based on the amount of chemotherapy agent delivered as indicated by the decreased level of fluid visible in the bag which is nearly at the peak or maximum drug concentration.

FIG. 4 is a view of the state of a process of chemotherapy-induced necrosis 410 in the targeted tumor or malignant lesion 420 of FIG. 1B.

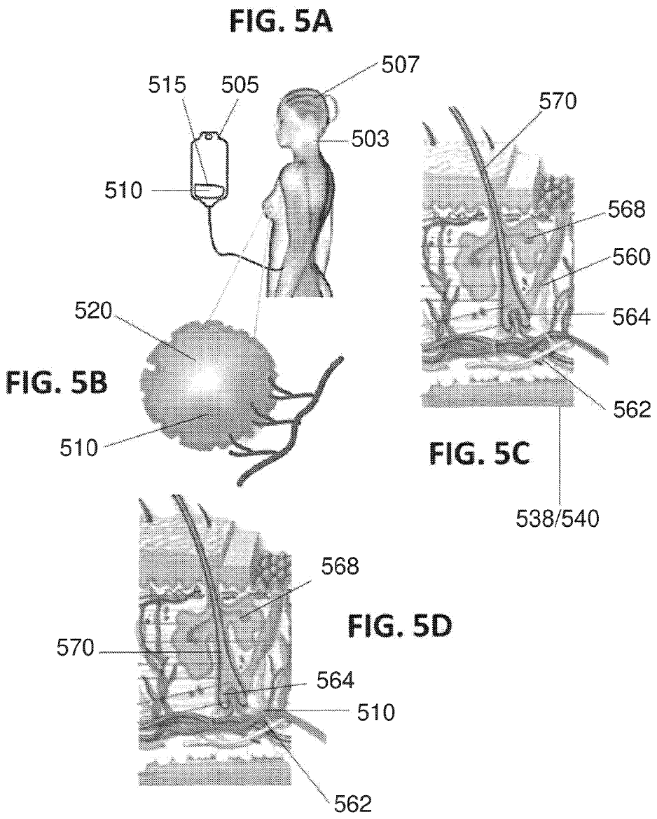

FIG. 5A is a view of the patient and the bag in FIG. 3A showing the amount delivered 515 and remaining of the chemotherapeutic agent 510 as indicated by the decreased level of fluid 515 in the IV bag 505. A Drug Concentration (mg/L) and Time (min) curve for the patient in FIG. 5A would be at, near or beyond the highest level. (See for example the peak level in FIG. 3B). Also shown are patient's hair 507 and head 503.

FIG. 5B is a view of the advancing state of the process of chemotherapy-induced necrosis 510 in the targeted tumor or malignant lesion 520, corresponding to tumor/malignant lesion 120 and 420 of FIGS. 1B and 4, respectively. A large amount of the chemotherapeutic agent 510 has been delivered to and absorbed by the tumor or lesion 520.

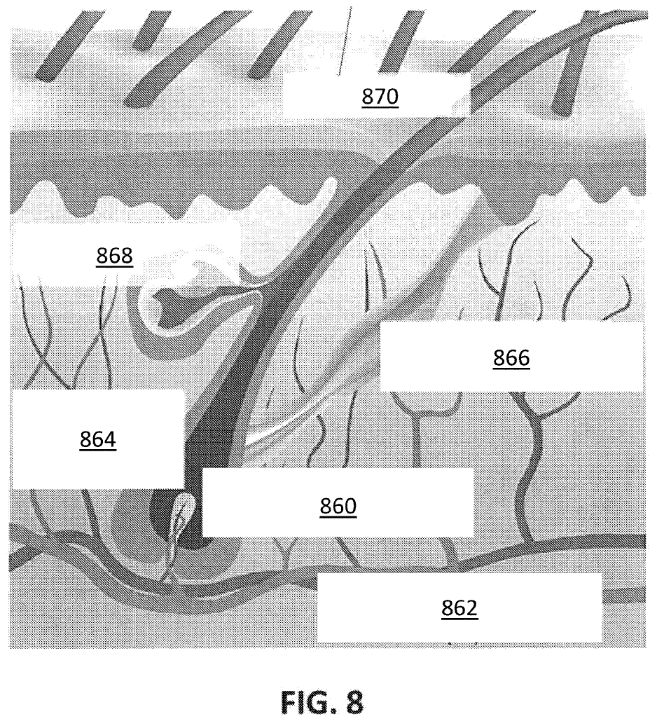

FIG. 5C is a cross section of the scalp and a hair follicle similar to FIG. 8. In this view, the vascular bed is shown before any chemotherapeutic agent 510 reaches the vasculature of the skull 560, scalp or vascular bed of the hair 568, bulb 564, hair shaft 570, as well as vasculature 562, periosteum 538, and bone 540.

FIG. 5D is a cross section of the scalp and a hair follicle vascular bed of FIG. 5C showing the infiltration and circulation of the chemotherapeutic agent 510 within the vasculature 562. Again shown are hair shaft 570, vascular bed of the hair 568, bulb 564, as well as vasculature 562.

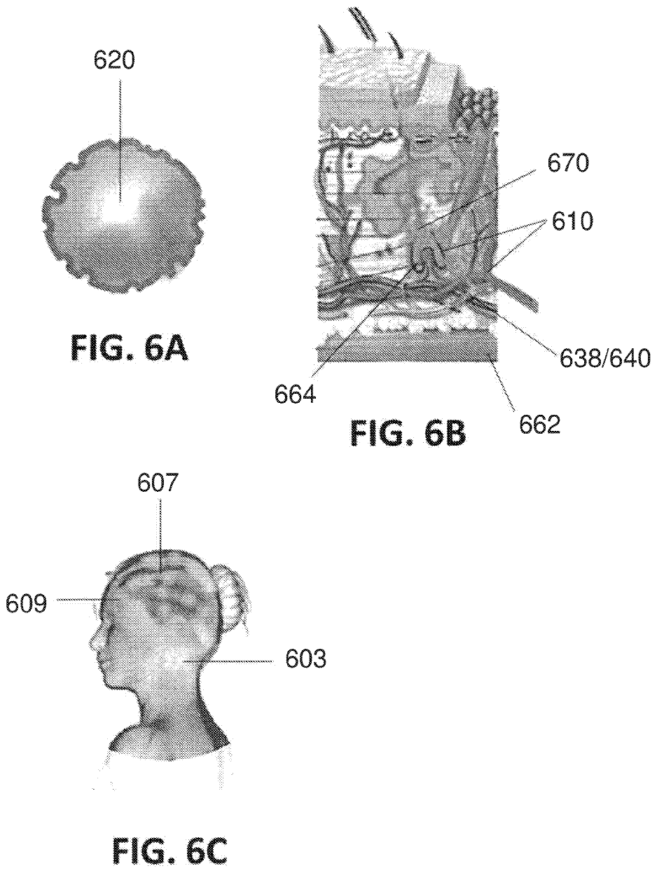

FIG. 6A is a view of the final state of chemotherapy-induced necrosis in the targeted tumor or malignant lesion 620, corresponding to tumor or malignant lesion 120, 420, and 520 of FIGS. 1B, 4 and 5B, respectively.

FIG. 6B is a view of the final state of wide spread chemotherapy-induced necrosis in the follicles of the patient which began in FIG. 5D. The vasculature 662 contains the chemotherapeutic agent 610 which has been delivered to and destroyed the bulb 664 and associated hair shaft 670. Introduction of the chemotherapeutic agent 610 into the vascular bed of the scalp and eyebrows produces in the patient one or more chemotherapy induced alopecia (CIA), chemotherapy drug-induced hair loss, hair follicle death and/or anagen effluvium. Also shown are periosteum 638 and bone 640.

FIG. 6C is a side view of the state of wide spread hair loss in the patient of FIGS. 1A, 3A, and 5A produced by one or a combination of chemotherapy-induced alopecia, hair loss, hair follicle death and/or anagen effluvium. The amount of hair 607 on the head 603 and eyebrows is less than the amounts in the prior views of FIGS. 1A, 3A and 5A. Also shown in eyebrow region 609.

The deleterious consequences described above may be mitigated or avoided to a large extent using an embodiment of a device and method described herein that impede or prevent chemotherapeutic agents from reaching the hair follicles. As used herein, vasocompression refers to a force applied to the head of a patient sufficient to overcome filling pressure or systolic pressure in the arterial supply to the scalp. Put another way, vasocompression achieved by the various embodiments and methods described herein block off the arterial blood supply to the scalp so that chemotherapeutic agents in the blood stream during chemotherapy are prevented from reaching the hair follicle blood supply.

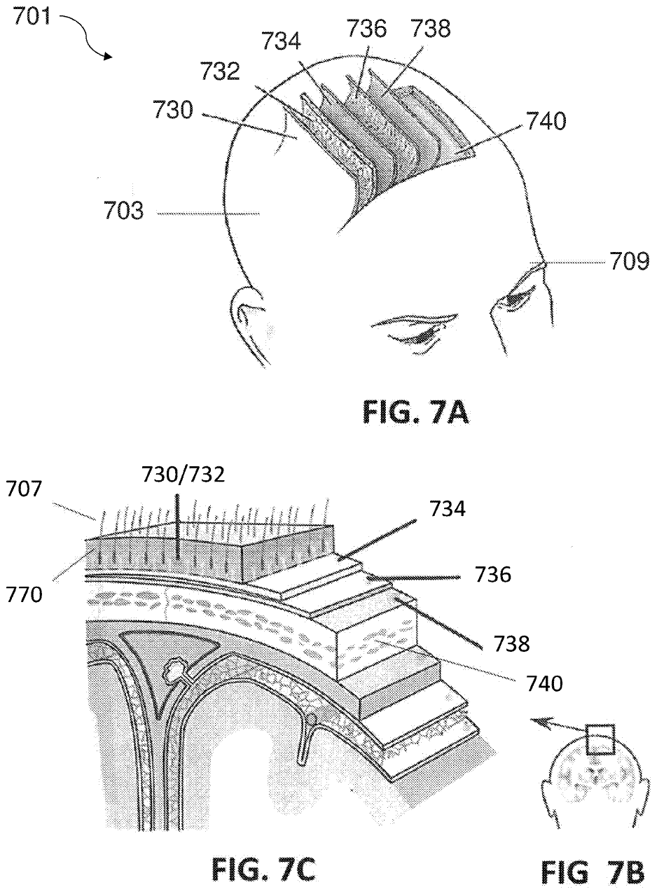

FIG. 7A is a perspective view of a head 703 of a patient 701 with pulled back portion of the skin 730 and scalp to reveal the layers of tissue over a bony portion of a skull. Beneath the skin 730 of head 703 is shown the connective tissue 732, aponeurosis or epicranial layer 734 and the loose areolar tissue 736. Below the loose areolar layer 736 is the pericranium 738 and then bone 740. An eyebrow region 709 is also shown.

FIG. 7B is a cross section of view of the pulled back portion of the skin and scalp of FIG. 7A.

FIG. 7C is a cross section of view of the indicated area of the skin and scalp of FIG. 7B. This view illustrates the relationship of the skin and dense connective tissue 730/732 to the epicranial aponeurosis 734, the loose areolar connective tissue 736 and the periosteum 738 and bone 740. Also shown is hair shaft 770 and scalp/hair 707.

FIG. 8 is a cross section of a healthy scalp and a hair follicle including the vascular bed. The blood supply 862 includes arteries and veins that supply nutrients to the individual hair follicles. The hair shaft 870 and bulb 864 are shown in relation to the dermal papilla 860 and blood supply 862. The hair shaft 870 is the portion of the hair visible above the scalp. The bulb 864 is the living part of the hair that surrounds the dermal papilla 860. Cells divide faster here than anywhere in the body. The dermal papilla 860 contain tiny blood vessels called capillaries that nourish the cells at the base of the hair follicle. The sebaceous gland 868 and arrector pili muscle 866 are also shown in this view. The sebaceous gland 868 produces sebum, which conditions the hair and skin. The arrector pili muscle 866 causes the hair to stand up and the sebaceous gland 868 to secrete oil.

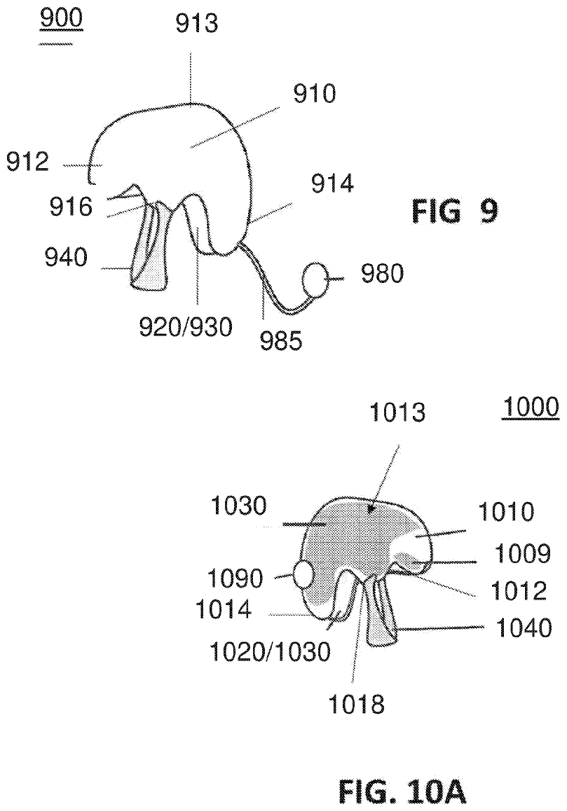

FIG. 9 is a perspective left side view of an embodiment of a head covering 900 for inducing vasocompression in the blood supply of a patient receiving chemotherapy, such as patient 100 from FIG. 1A. This view also shows a shell 910 along with a front edge 912, a top 913, a left side edge 916 and a back edge 914. Also shown in this view is portion of the head covering interior with a portion of the liner 920 and one or more vasocompression elements 930 visible. The one or more vasocompression elements 930 are connected to an external pump or controller 980 using a suitable connector 985. The pump or controller 980 is used to execute the desired vasocompression therapy to the patient using the one or more vasocompression elements 930 in the head covering 900. A submandibular strap connected to the shell 910 is also visible in this view. The head covering 900 has a shell 910 adapted and configured to deliver vasocompression to the scalp with a front edge 912 selected to provide vasocompression to the eyebrows. In one aspect, one or more vasocompressive elements 930 may be placed continuously along the liner or interior of the shell 910 to treat the scalp/hair 107 and eyebrows 109 (from FIG. 1A) and the space on the forehead between the hair line and the eyebrows. Additionally or optionally, separate vasocompression elements 930 are used for the eyebrows and the hair with no elements 930 in the forehead region between the hair line and the eyebrows. Also shown in head covering retention system 940.

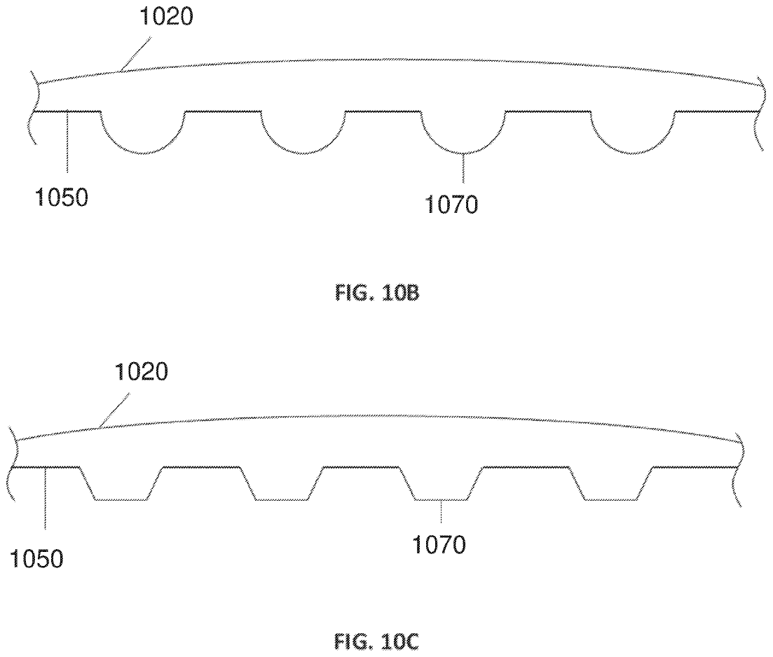

FIG. 10A is a perspective right side view and partial interior view of an embodiment of a head covering 1000 for inducing vasocompression in the blood supply of a patient 100 from FIG. 1A receiving chemotherapy. This view also shows a shell 1010 with a top 1013, a front edge 1012, a back edge 1014 and a right-side edge 1018. The shell 1010 is illustrated as transparent to show the location of the vasocompression elements 1030 aligned to delivery vasocompression therapy to the hair/scalp 107 and eyebrows 109 of the patient 100 from FIG. 1A. Also shown in this view is an interior portion of the liner 1020 adjacent to the left side edge 916 from FIG. 9. In this embodiment of the head covering, the pump or controller 1090 is mounted directly onto the head covering 1000—such as to a portion of the shell 1010. The pump or controller 1090—like the pump or controller 980 (from FIG. 9)—is appropriately connected to the one or more vasocompression elements 1030 in the head covering 1000. The mounted pump or controller 1090 is used to execute the desired vasocompression therapy to the patient using the one or more vasocompression elements 1030 in the head covering 1000. A submandibular strap 1040 connected to the shell 1010 is also visible in this view. The vasocompression element 1030 illustrated is an inwardly expanding manifold where inwardly is toward scalp—away from shell 1010. The head covering 1000 is configured to deliver vasocompression to the scalp 107 and eyebrows 109 (from FIG. 1A) as can be seen by the size, orientation and location of the vasocompression element 1030 positioned adjacent to the scalp and another separate or spaced apart vasocompression element 1030 positioned for engagement with the eyebrows 109 (from FIG. 1A).

FIG. 10B is a cross section view of a portion of a liner 1020 having a vasocompression element/expandable bladder 1050B with one or more raised features 1070. In this illustrative embodiment, the raised features 170 have a rounded shape facing the patient who is wearing the head covering 100.

FIG. 10C is a cross section view of a portion of a liner 1020 having a vasocompression element/expandable bladder 1050B with one or more raised features 1070. In this illustrative embodiment, the raised features 1070 have a rectangular pyramid shape with a flat top section facing the patient who is wearing the head covering, for example head covering 900 from FIG. 9 or head covering 1000 from FIG. 10A.

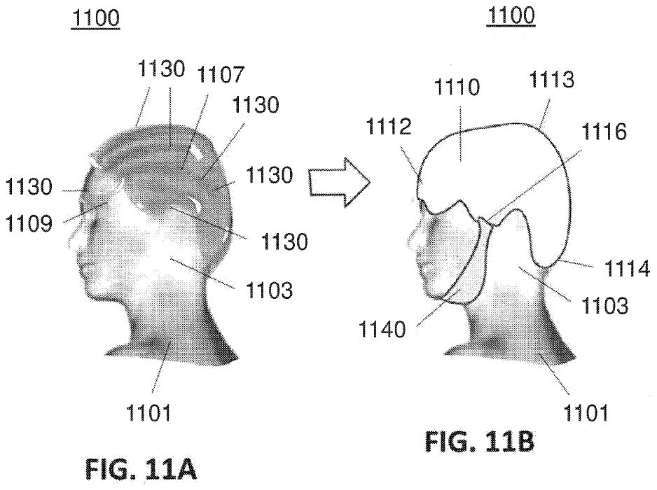

FIG. 11A is a left side view of a patient's head 1103 who is wearing a vasocompression element used to deliver vasocompression in the blood supply of the patient while receiving chemotherapy. The outer shell 1110 of the head covering 1100 is removed in this view (but can be seen in the view of FIG. 11B). An array of different sized and shaped vasocompression elements 1130 are visible in this view. Additionally or optionally, the one or more elements 1130 may be configured as an inflatable liner which is connected to a pump or controller 980/1090 (not visible in this view, see FIGS. 9 and 10A, respectively). In this configuration, the inflatable liner vasocompression elements 1130 are configured to deliver vasocompression to the scalp 107 and eyebrows 109 from FIG. 1A.

FIG. 11B is a left side view of the patient in FIG. 11A showing a complete head covering 1100 with the shell 1110 in place. The head covering 1100 is held to the head 1103 using an exemplary submandibular strap 1140. FIG. 11B illustrates an exemplary position of the head covering 1100 on the head 1103 for inducing vasocompression in the blood supply of a patient 1101 receiving chemotherapy using the vasocompression element configuration of FIG. 11A. In one aspect, the head covering 1110 will deliver vasocompression for protection of the scalp 1107 and eyebrows 1109.

The head coverings 900, 1000, and 1100 illustrated and described in FIGS. 9, 10, 11A and 11B, respectively, are subject to a number of variations and alternatives. Turning now to a number of additional details of the features and operations of the various head covering embodiments.

In one embodiment, there is provided a head covering 900 for inducing vasocompression in the blood supply of a patient receiving chemotherapy having a shell 910 having a front edge, a back edge, a top, a left side edge and a right side edge. There is a liner within the shell shaped to at least partially accommodate the hair of the patient. There is also included at least one vasocompression element within the outer shell and a head covering retention system coupled to the shell.

In one aspect, the head covering retention system 940 (from FIG. 9) may include a submandibular strap connected to the shell 910. Optionally, the head covering retention system 940 may include one or more of a rear strap, a front strap or a neck strap connected to the shell 910. In some embodiments, the shell retention system 940 comprises one or more adjustment elements. Optionally, the head covering retention system 940 may include a hook and loop fastener, a buckle, a quick release mechanism or an adjustment feature.

In additional aspects, as shown in FIGS. 10B-10C, the at least one vasocompression element is an expandable bladder 1050B within the liner 1020. In an additional aspect, the at least one vasocompression element is coupled to the shell 1010. There may also be a pressure source (not shown) in communication with the expandable bladder 1050B. Additionally or optionally, in FIG. 9, there may be provided a continuous perimeter (not shown) connecting the front edge 912, the back edge 914, the left side edge 916 and the right side edge 918. Additionally, in one embodiment the head covering 900 is a continuous structure from the occiput area to the eyebrows or to the forehead. Still further, there is provided a structure or an inflatable structure to compress the eyebrows for a vasocompression effect on the eyebrows. It is to be appreciated that the pressure source may be used alone or as part of a control system as configured according to control system 980 and connector 985 in FIG. 9 or according to control system 1090 as configured in FIG. 10A. In one embodiment, the pressure source (not shown) is in communication with the components of the head covering 900 using a suitable connector 985 as shown in FIG. 9. In another embodiment, the pressure source (not shown) is mounted on the head covering 1000 as shown in the configuration of the control system 1090 in FIG. 10A.

In additional embodiments, the at least one vasocompression element includes an expandable feature responsive to a pressure source to expand away from the shell. There may also be provided an expandable layer coupled to the liner wherein the at least one vasocompression element is on the expandable layer. Additionally or optionally, the at least one vasocompression element includes one or more raised features. The liner within the shell shaped to at least partially accommodate the hair of the patient may also include one or more features on the liner to engage with the hair of the patient. Still further, the liner or the at least one vasocompression element may be smooth, textured, have irregular shapes of include an array of evenly distributed protrusions having straight or circular semi-rigid, flexible or inflatable structures.

In still other aspects, there is included a gas source in communication with the liner or the at least one vasocompression element for inflation of a portion of the liner or a portion of the at least one vasocompression element. This system may also include at least one relief valve to prevent over pressurization of an inflatable element of the head covering 1000. In various embodiments, the gas source is a manual pump mechanism, a bulb, an electric pump, or pressurized tank, cartridge or reservoir. Optionally, the gas source may be worn or carried by the patient. Still further, the head covering 1000 may include fittings or connectors for use with house gas, clean dry air or other building supplied gas source. Additionally or optionally, there may also be provided a control system, such as control system 980 or control system 1090 configured for adjusting the flow of gas from the gas source to maintain a desired pressure within the head covering 900 and 1000, from FIGS. 9 and 10A, respectively.

In still further embodiments, the control system 980 or the control system 1090 may be a gas source and control system adapted and configured to include other additional capabilities such as these described herein. The desired pressure within the head covering 1000 is sufficient to produce a vasocompression result while minimizing discomfort or side effects to the patient or harm to surrounding structures. There may also be included a cooling gas source in communication with the liner or the at least one vasocompression element wherein a portion of the liner or a portion of the at least one vasocompression element is adapted to provide cooling to an interior portion of the head covering 1000. There may also be included one or more pressure sensors within an interior portion of the head covering 1000. The one or more pressure sensors may be selected and provided appropriately to generate a signal of an indication of pressure to the control system 980/1090. Still further, the one or more pressure sensors are adapted to indicate an amount of pressure applied to a patient undergoing chemotherapy when the patient is wearing the head covering 1000. Additionally, the one or more sensors may be configured to measure skin perfusion (O₂ saturation) or Doppler blood flow monitor or infra-red pulse oximeter sensor. The sensors described herein may also be adapted and configured to determine blood flow rates in the vasculature receiving vasocompression to determine that the flow has stopped or slowed sufficiently so that to prevent flow of blood containing a chemotherapeutic agent. In one aspect, the detection of substantially reduced blood flow or stopped blood flow could also be used as a trigger within the control system 180/190 to increase the amount of vasocompression by an additional margin to increase confidence that the amount of vasocompression applied is sufficient. This additional margin of vasocompression may be included in a feedback loop performed by the control system 180/190 that measures amount of blood flow, increases vasocompression, measures blood flow to determine if slowed or stopped and then increasing vasocompression level as needed. Thereafter, an additional step of increasing vasocompression level after detection of stopped blood flow may be added as a safety margin to ensure temporary and reversible vasocompression induced vascular closure.

Additionally, the head covering 1000 may be coupled to a timer and a control system 180/190 adapted to provide an automatic deflation protocol when the timer has elapsed or to cease operation of any active vasocompression element. In a variation, the head covering 1000 may be coupled to a timer and a control system 980/1090 adapted for programmed automatic inflation and deflation of the liner or the one or more vasoconstriction elements or the activation and deactivation of the one or more vasoconstriction elements. The system 980/1090 may also include a display and Bluetooth, near field or other wireless or wired communication capabilities. Still further, the system may communicate status, programming, patient information or other aspects of vasocompression therapy or operations to a mobile phone, network, remote network or health information system. In still other aspects, the display may be positioned on the head covering 1000 for providing information to a health care provider or the patient. Additionally or optionally, a display may be located on, in, within or integral to a portion of the shell 1010. An embodiment of the display may be on a movable mount to allow viewing of the display by the patient using the head covering. Additionally or optionally, the head covering may be adapted with a suitable mount, connector or connectivity to allow a smart phone or other separate electronic device to be mounted to the head covering and configured for electronic communications, interactions and control of the components of the head covering. In additional aspects, the head covering 1000 may also include ear buds, headphones, speakers or other audio components adjacent to the ears of the patient. In these embodiments, the system is also configured to provide noise cancellation, music, media or other entertainment to the patient while undergoing chemotherapy or using the head covering 1000.

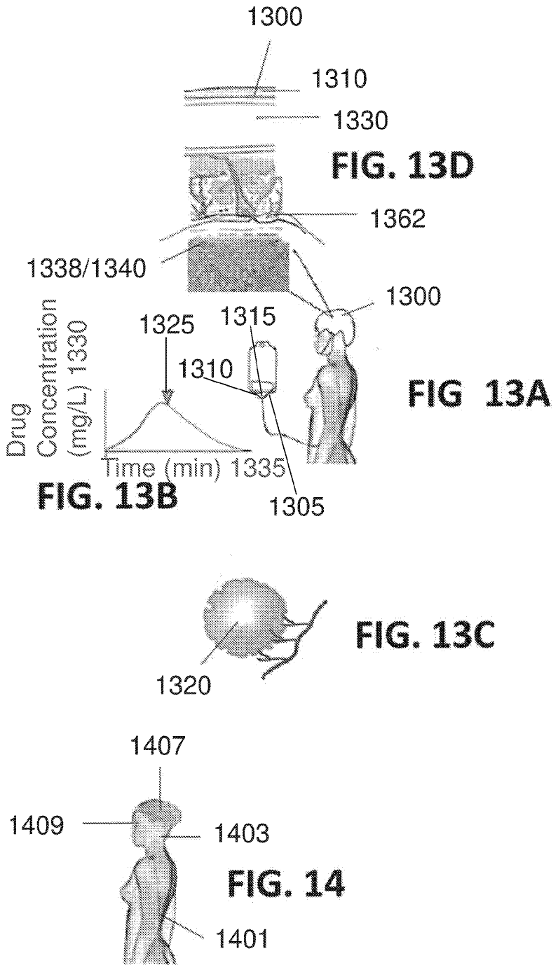

Additionally, with regard to the head covering 100 being operated by a timer sequence of an embodiment of the control system 980/1090, the timing may be adapted to correspond to the expected half-life or toxicity level of the chemotherapy agent is use. In this way, the initiation of vasocompression therapy and the duration of the vasocompression therapy may be related to the introduction of the chemotherapeutic agent as well as the expected duration of the vasocompression therapy to correspond to the lingering toxicity in the blood of the residual chemotherapeutic agent. Considering the drug concentration profiles in FIGS. 3B and 13B, the duration of the vasocompression therapy would be selected to that time duration where the drug concentration level is lower than the peak level and approaching zero or pre-chemotherapy levels in the blood. This amount may vary by the patient as well as the specific toxicity profiles and half-lives of specific chemotherapeutic agents in use. As such, the timing function of the controller 980/1090 may provide an automatic deflation protocol when the timer has elapsed which was selected to address the specifics of the patient and the agent used. Optionally, an additional timing margin may be added to provide additional confidence that the toxic level of chemotherapeutic agent in the blood poses no further thread or is a significantly reduced threat to the health of the scalp and hair structures (FIG. 8). Additionally or optionally, once the timer has elapsed or to cease operation of any active vasocompression element, the control system may stage or stagger the cessation of vasocompression therapy to sequence the re-introduction of blood flow after vasocompression therapy. In a variation, the head covering 100 may be coupled to a timer and a control system 980/1090 adapted for programmed automatic inflation and deflation of the liner or the one or more vasoconstriction elements or the activation and deactivation of the one or more vasoconstriction elements in accordance with the guidelines discussed above.

In still other embodiments of a head covering 1000 having a liner, the liner may be a rigid or semi-elastic layer configured for an exact fit for the patient to fit exactly the shape of the patient's head and hair.

The various hair covering embodiments described herein may also one or more selective pressure points positioned to compress one or more vessels of the cranial circulatory system of a patient. Additionally, other embodiments may include a vibrating element coupled to the liner or the at least one vasocompression element. In still further embodiments, the liner 1020 and the at least one vasocompression element 1030 are sized, shaped, or positioned relative to the head covering 1000 based on the hair style of the patient.

In yet another alternative embodiment of a head covering 1000, there is a cap having a front edge, a back edge, a top, a left side edge and a right side edge. There is a liner within the cap shaped to at least partially accommodate the hair of the patient. There is at least one vasocompression element within the cap. In one alternative, the cap comprises a flexible structure, a partially flexible structure, a combination of semi-rigid structure and flexible structure or an adjustable structure sized to remain in place on the head when the at least on vasocompression element is active.

In still other various alternative embodiments, the head covering 1000 with the cap form factor may also be adapted and configured to include any of the above mentioned alternative, optional and additional variations and embodiments.

In some embodiments, one or more vasocompression elements are urged into contact with the scalp via springs, mechanical linkage, electromechanical drive, or electromagnetic arrangement.

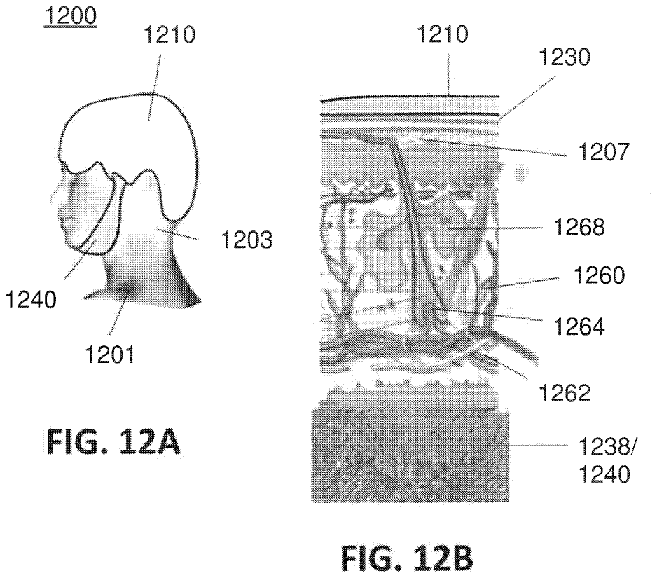

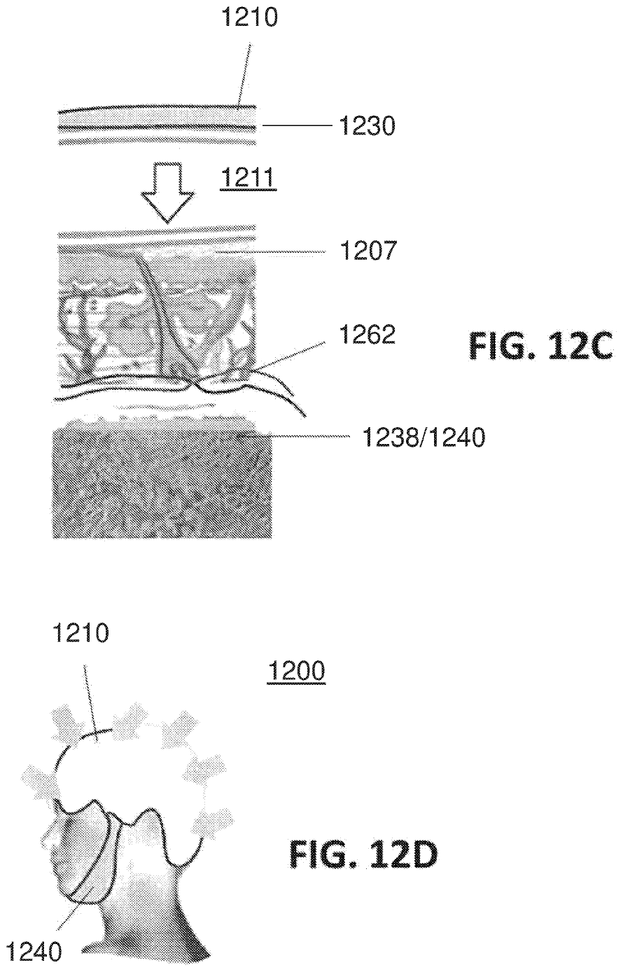

Turning now to FIGS. 12A-15 which will be used to describe an exemplary method of providing vasocompression to a patient undergoing chemotherapy. It is to be appreciated that the methods and variations thereof may be modified for implementation using any of the above described head covering 1000 embodiments or vasocompression elements 1030.

FIG. 12A is a left side view of a patient's head who is wearing a head covering 1200 including at least one vasocompression element such as vasocompression element 1230 from FIG. 12B used to deliver vasocompression. This is the state of the patient prior to or shortly after initiation of the patient receiving chemotherapy. A submandibular strap 1240 connected to a shell 1210 of the head covering 1200 is also visible in this view. The head covering 1200 is configured to deliver vasocompression to the scalp 1207 and eyebrows 109 (from FIG. 1) of the patient.

FIG. 12B is a cross section of a portion of the hair 1207, scalp and hair follicles of the patient in FIG. 12A. This view shows the hair 1207 pressed against the scalp when the head covering 1200 in donned (FIG. 12A) and prior to the delivery of vasocompression. Also shown is vasculature for skull 1260, scalp 1207 or vascular bed of the hair 1268, bulb 1264, as well as vasculature 1262, periosteum 1238/bone 1240, vasocompression element 1230 and shell 1210.

FIG. 12C is a cross section of a portion of the hair, scalp and hair follicles of the patient in FIG. 12B. There is a rigid outer shell 1210. This view shows the expansion of one or more vasocompression elements 1230 in the head covering 1200 (inwardly expanding manifold in the direction of the arrow 1211) causing vasocompression within the vascular bed 1262 of the follicle. Also shown is scalp 1207 and periosteum 1238/bone 1240.

FIG. 12D is an exterior view of the patient 1200 undergoing chemotherapy while vasocompression is being delivered by the head covering 1200. A submandibular strap 1240 connected to a shell 1210 of the head covering 1200 is also visible in this view. The arrows indicating that vasocompression may be simultaneously or sequentially applied to numerous portions of the scalp 1207 including the eyebrows 109 (from FIG. 1A).

FIG. 13A is a view of the patient of FIGS. 12A-12D receiving vasocompression from the head covering during a chemotherapy session which is nearly completed as indicated by the nearly empty IV bag 1305 of chemotherapeutic agent 1310, and the amount of fluid in the bag 1315. Also shown is head covering 1300.

FIG. 13B is the graph showing an expected concentration curve of the chemotherapeutic agent 1310 in the patient's blood stream as a function of Drug Concentration (mg/L) 1330 and Time (min) 1335 for the status of the patient in FIG. 13A. The triangle 1325 marks the estimated concentration based on the amount of chemotherapy agent delivered as indicated by the decreased level of fluid visible in the bag of FIG. 13A. The arrow indicates that the drug concentration amount is nearly at or just beyond the peak or maximum drug concentration.

FIG. 13C is a view of the advancing state of the process of chemotherapy-induced necrosis in the targeted tumor or malignant lesion 1320 of the patient of FIGS. 12A, 12D and 13A who has been receiving vasocompression during the delivery of the chemotherapeutic agent.

FIG. 13D is a cross section of the head covering 1300, scalp and a hair follicle of the patient of FIGS. 12A, 12D and 13A who has been receiving vasocompression during the delivery of the chemotherapeutic agent 1310 (FIG. 13B). This view shows that the vascular bed is completely free or substantially free of any chemotherapeutic agent 1310 or that the level of chemotherapeutic agent 1310 that reaches the vascular bed is maintained below a level to harm the follicles. As shown here, there is head covering 1300, shell 1310, manifold 1330, as well as vascular bed 1362, periosteum 1338/bone 1340.

In contrast to the disastrous damage experiences by the unprotected patient in FIG. 6B, the patient who received vasocompression during chemotherapy has closed off vasculature 662 preventing the chemotherapeutic agent 10 from being delivered to or damaging the bulb 664 and associated hair shaft 670. As a result of the advantageous prevention of the introduction of the chemotherapeutic agent into the vascular bed of the scalp and eyebrows, the patient who receives vasocompression as described herein in combination with chemotherapy avoids one or more of chemotherapy induced alopecia (CIA), chemotherapy drug-induced hair loss, hair follicle death and/or anagen effluvium.

FIG. 14 is a side view of the patient 1401 of FIG. 13A at a time period after the delivery of chemotherapy with vasocompression. The patient has maintained hair 1407 on both the head 1403 and the eyebrows 1409 as a result of the beneficial effects of vasocompression.

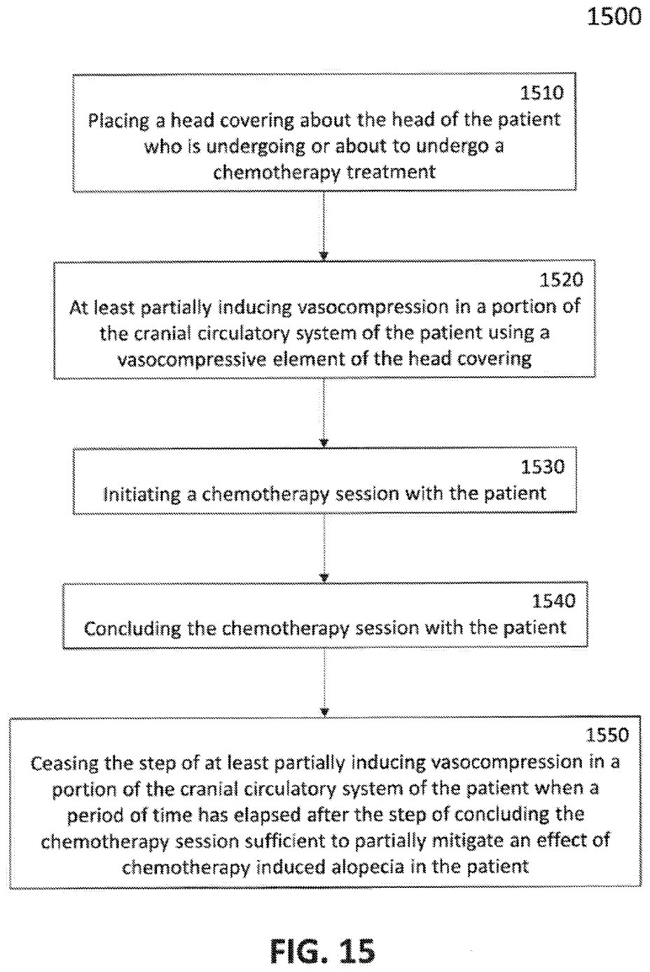

FIG. 15 is a flow chart of a representative method 1500 for providing vasocompression to a patient undergoing a chemotherapy treatment.

The representative method of inducing vasocompression in a cranial circulatory system of a patient receiving chemotherapy begins by placing a head covering 100 about the head of the patient (step 1510).

Next, there is a step of at least partially inducing vasocompression in a portion of the cranial circulatory system of the patient using a vasocompression element of the head covering 100 (step 1520).

Next, there is a step of initiating a chemotherapy session with the patient (step 1530).

Next, there is a step of concluding the chemotherapy session with the patient (step 1540).

Finally, there is a step of ceasing the step of at least partially inducing vasocompression in a portion of the cranial circulatory system of the patient when a period of time has elapsed after the step of concluding the chemotherapy session sufficient to partially mitigate an effect of chemotherapy induced alopecia in the patient (step 1550).

Optionally or additionally, the step of inducing vasocompression in a portion of the cranial circulatory system may be adjusted before, during or after the steps of initiating or concluding the chemotherapy session.

The method 1500 may also include engaging a head covering retention system 940 to immobilize the head covering 900 (from FIG. 9) relative to the head of the patient.

The period of time that has elapsed after the step of concluding the chemotherapy session is more than 15 minutes, 30 minutes, 45 minutes, 1 hour, 2 hours, 3 hours or a period of time related to when the level of drug concentration in the patient's blood stream is below a level to harm the hair follicles.

In an additional aspect of the method, as shown in FIG. 9, the vasocompression element 930 of the head covering 900 is a liner 920 wherein a step of expanding the liner or a step of contracting the liner is performed during the step of at least partially inducing vasocompression in a portion of the cranial circulatory system of the patient. Additionally or optionally, there is an increase or a decrease in the amount of vasocompression during the step of expanding the liner corresponding to an increase of the force of the liner against the scalp of the patient.

In an additional aspect of the method, there is provided a vasocompression element of the head covering that is a liner having one or more features wherein a step of expanding the liner or a step of contracting the liner is performed during the step of at least partially inducing vasocompression in a portion of the cranial circulatory system of the patient. In a still further aspect, there is an increase in the amount of vasocompression during the step of expanding the liner corresponding to an increase of the force of the one or more features on the liner against the scalp of the patient. Additionally or optionally, there a decrease in the amount of vasocompression during the step of contracting the liner corresponding to a decrease of the force of the one or more features on the liner against the scalp of the patient.

In yet another variation on the method, the vasocompression element 930 of the head covering is an inflatable bladder wherein the degree of vasocompression during the step of at least partially inducing vasocompression in a portion of the cranial circulatory system of the patient corresponds at least in part to the amount of pressure within the inflatable bladder.

In yet another variation on the method, the vasocompression element 930 of the head covering is an inflatable bladder wherein the degree of vasocompression during the step of at least partially inducing vasocompression in a portion of the cranial circulatory system of the patient is increased or decreased by raising or lowering the pressure in the inflatable bladder against the skull.

In yet another variation, the vasocompression element of the head covering is one or more features arranged on an interior surface of the head covering which are urged into contact with a portion of the patient's scalp having hair follicles during the step of at least partially inducing vasocompression in a portion of the cranial circulatory system of the patient.

Another variation includes a vasocompression element of the head covering is one or more features arranged on an interior surface of the head covering which correspond to an arrangement of the patient's hair, wherein the one of more features act on the arrangement of the patient's hair to further the performance of the step of least partially inducing vasocompression in a portion of the cranial circulatory system of the patient.

In still another variation of the method, during the at least partially inducing vasocompression step the vasocompression element of the head covering compresses a portion of the scalp containing a plurality of hair follicles against a portion of the cranium to at least partially vasocompress the blood supply to the plurality of hair follicles. Additionally or optionally, there is provided a step wherein the portion of the cranium is one or more of a superior temporal line, an inferior temporal line, a parietal bone, a squamous suture, a temporal bone, a lambdoid suture, an occipital bone, a mastoid process, a sphenoid, a portion in proximity to the supraorbital foramen, a frontal bone, a coronal suture

In another alternative version of the method, during the at least partially inducing vasocompression step, the vasocompression element of the head covering is positioned to at least partially vasocompress an artery or a vein of the cranial circulatory system of the patient by compressing the artery or the vein against a portion of the cranium. Still further, the artery or the vein of the cranial circulatory system is a branch of an external carotid artery or a branch of the internal carotid artery, the artery or the vein of the cranial circulatory system is an ophthalmic artery, the artery or the vein of the cranial circulatory system is a superficial temporal artery, the artery or the vein of the cranial circulatory system is a posterior auricular artery, the artery or the vein of the cranial circulatory system is an occipital artery, the artery or the vein of the cranial circulatory system is a supraorbital artery or a supratrochlear artery Additionally, optionally or in combination, the portion of the cranium is one or more of a superior temporal line, an inferior temporal line, a parietal bone, a squamous suture, a temporal bone, a lambdoid suture, an occipital bone, a mastoid process, a sphenoid, a portion in proximity to the supraorbital foramen, a frontal bone, a coronal suture. Referring to FIG. 10A, it is to be appreciated that the size, shape and orientation in use of the shell 1010 along with the size, shape, overall position and relative positions of a front edge 1012, a back edge 1014, a top 1013, a left side edge 916 (from FIG. 9) and a right side edge 1018 of a shell 1010 may be advantageously selected alone or in any combination with one or more vasocompression element embodiments described herein to selectively apply active and controllable vasocompression to one or more of the arteries or veins of the cranial circulatory system listed above. In one specific example, considering the scalp receives a rich arterial supply via the external carotid artery and the ophthalmic artery (a branch of the internal carotid), there is provided an embodiment of a vasocompression element or arrangement of elements that will compress the feeding vessels at a higher pressure (not like a torniquet but rather a direct point pressure) than the pressure otherwise applied to the regions of the scalp/hair or eyebrows.

In an additional variation, the method includes the step of at least partially inducing vasocompression in a portion of the cranial circulatory system results from compression of a portion of the scalp containing a blood vessel of the cranial circulatory system of the patient against a portion of the periosteum of the patient.

In an additional variation, the method includes the step of at least partially inducing vasocompression in a portion of the cranial circulatory system is produced by interaction between the vasocompression element of the head covering and a blood vessel of the cranial circulatory system of the patient.

In still another variation, there is a step of at least partially inducing vasocompression in a portion of the cranial circulatory system further comprising compressing a portion of the scalp against a portion of the periosteum to induce vasocompression in a portion of a vascular bed of a hair follicle.

In another alternative version of the method, there is a step wherein the vasocompression element of the head covering is positioned with respect to the patient's head wherein during the engaging step the vasocompression element is positioned in proximity a specific vein or a specific artery of the cranial circulatory system of the patient to at least partially vasocompress the specific vein or the specific artery.

Another alternative of the method includes delivery of a vasoconstrictor medication to the patient before, during or after any of the above mentioned methods. In one aspect, the vasoconstrictor medication is one of an alpha-adrenoceptor agonist, a vasopressin analog, an epinephrine, a norepinephrine, a phenylephrine, a dopamine, a dobutamine, a migraine medication, a headache medication, a serotonin 5-hydroxytryptamine agonist, and a triptans.

Additionally, the above methods and devices and all variations thereof may be used to delivery vasocompression while a patient is receiving chemotherapy by an injection, an infusion, a targeted therapy, a hormone therapy or an immunotherapy. Still further, these methods and devices of providing vasocompression may be employed before or while the patient is receiving chemotherapy by an intravenous method, an intrathecal method, an intra-arterial method, an intracavitary method, an intramuscular method, an intralesional method, or an intravesical method. Additionally or optionally, because these methods and devices of providing vasocompression are compact, portable and mobile with the patient, the methods and devices may be readily employed regardless of whether the patient is receiving chemotherapy in a home, a health care provider office, a clinic, an outpatient infusion center, a hospital infusion center, or a hospital in-patient room.

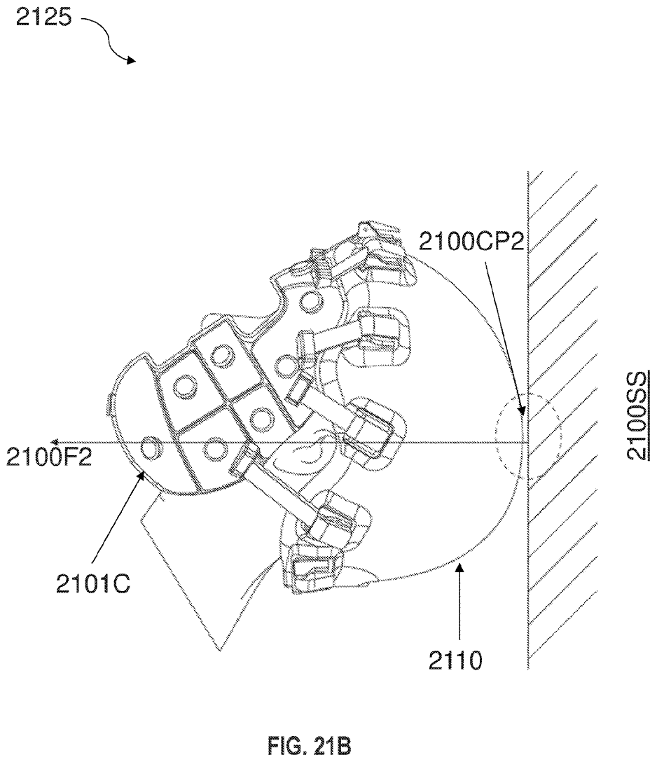

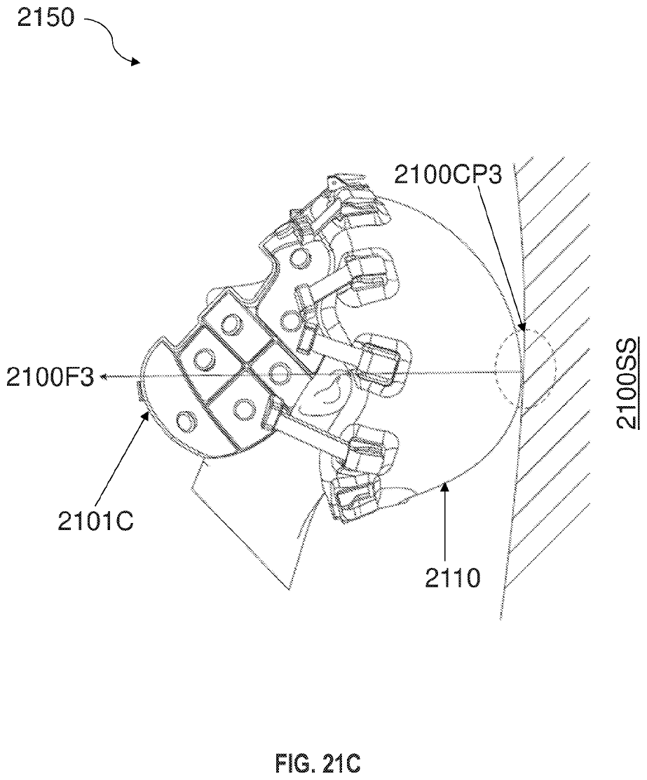

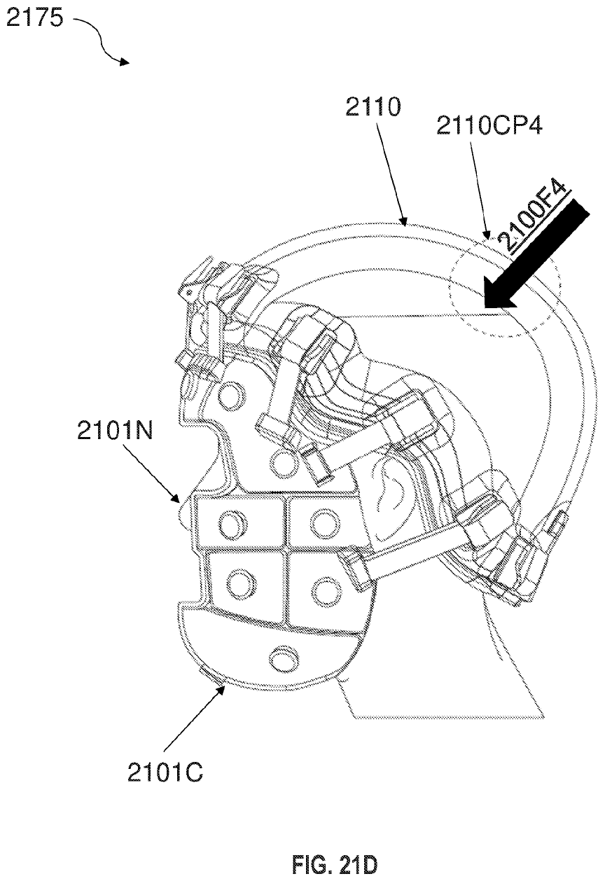

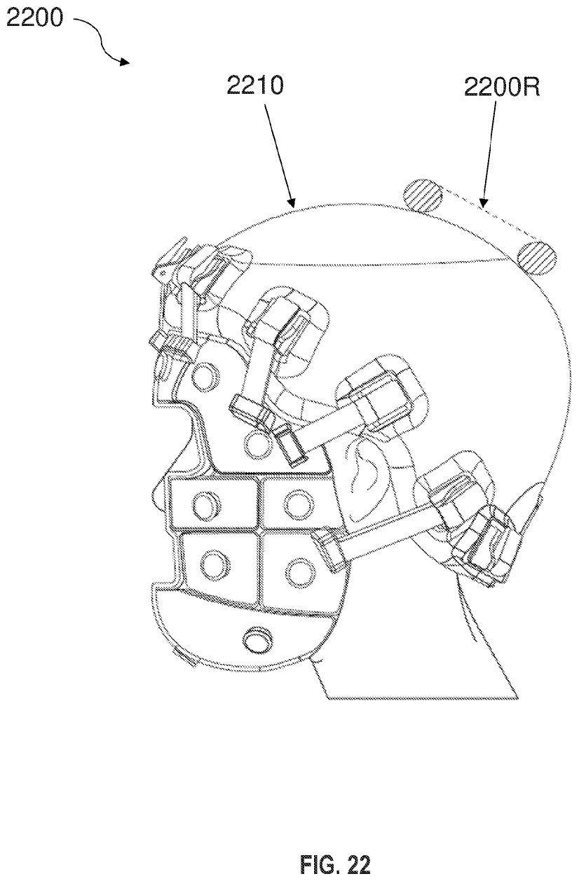

In another alternative version of the method 1500, there is provided a step wherein a counter force is applied to the head covering which is used to offset the forces introduced to the head of the patient as a result of the vasocompression therapy. The counter force may be applied directly to the shell of the head covering in a suitable location based on patient position and the desired vector of the counter force as illustrated and described with regard to FIGS. 21A-21D. Additionally or optionally, a support structure may be placed on, releasably coupled to the shell of the head covering in order to facilitate the repeatable or controllable application of a generated counter force as illustrated and described with regard to the embodiments of FIGS. 22-26. The method and amount of the application of the vasocompression counterforce may vary during a vasocompression therapy session. The amount of the counter force and direction of application of the counter force may also change during a session or be different during subsequent sessions based on patient comfort and the vasocompression forces generated during a chemotherapy session. In one aspect, a criteria in the selection and application of a specific embodiment of a vasocompression counter force is patient preference and comfort.

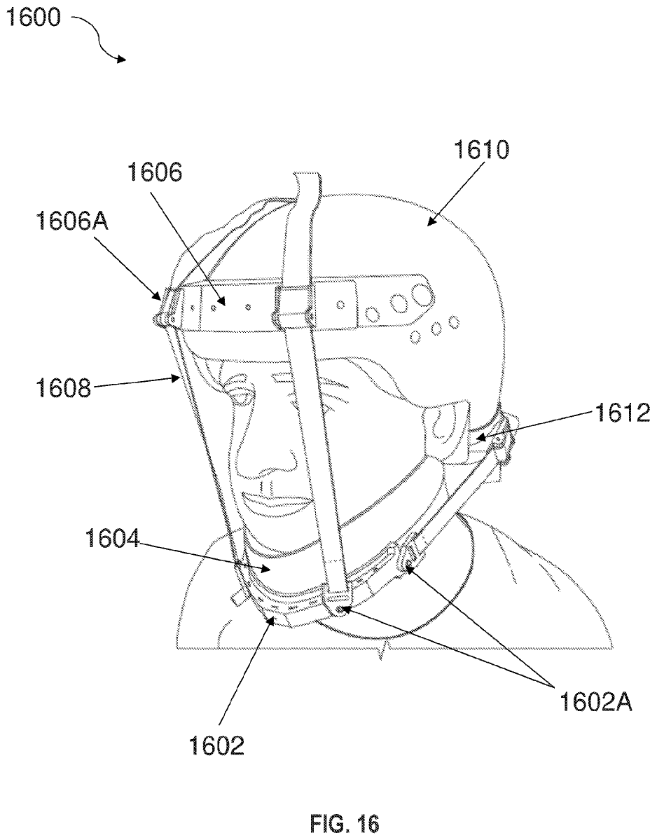

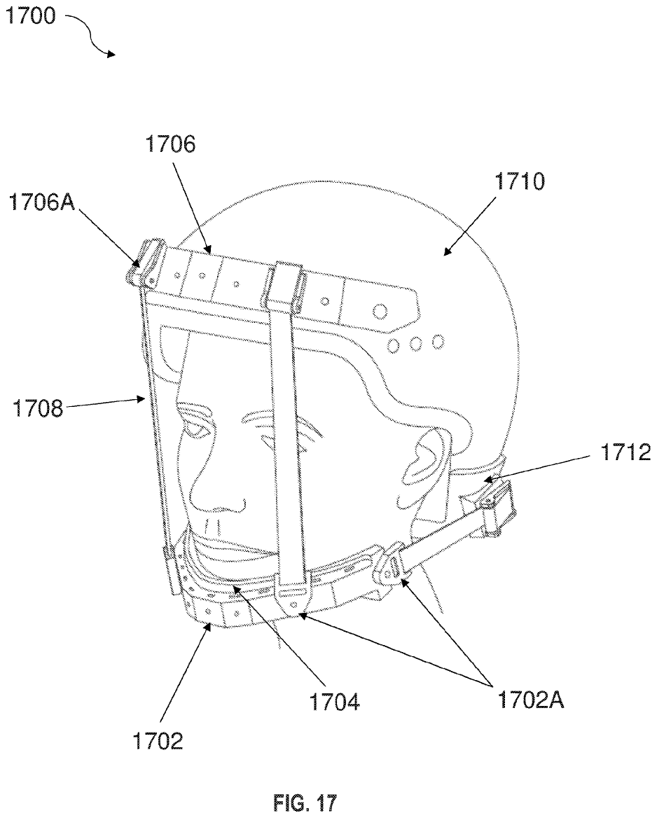

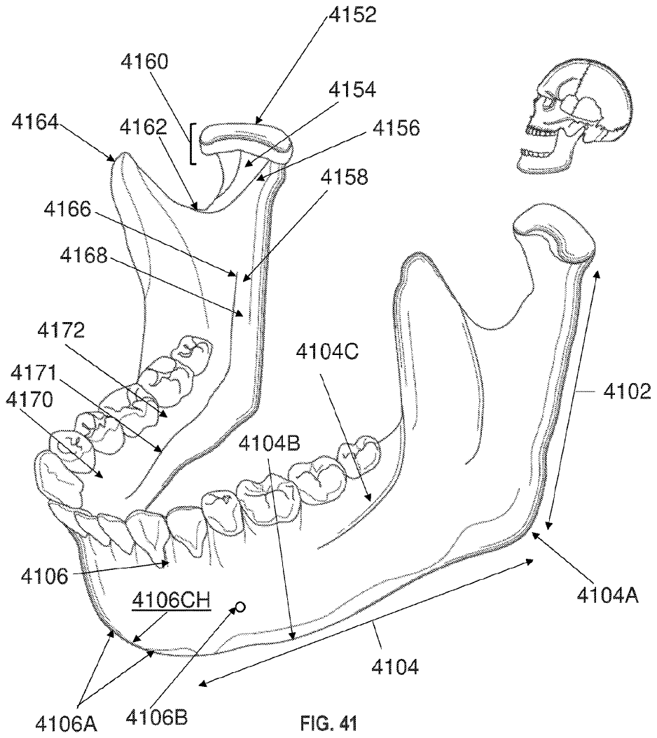

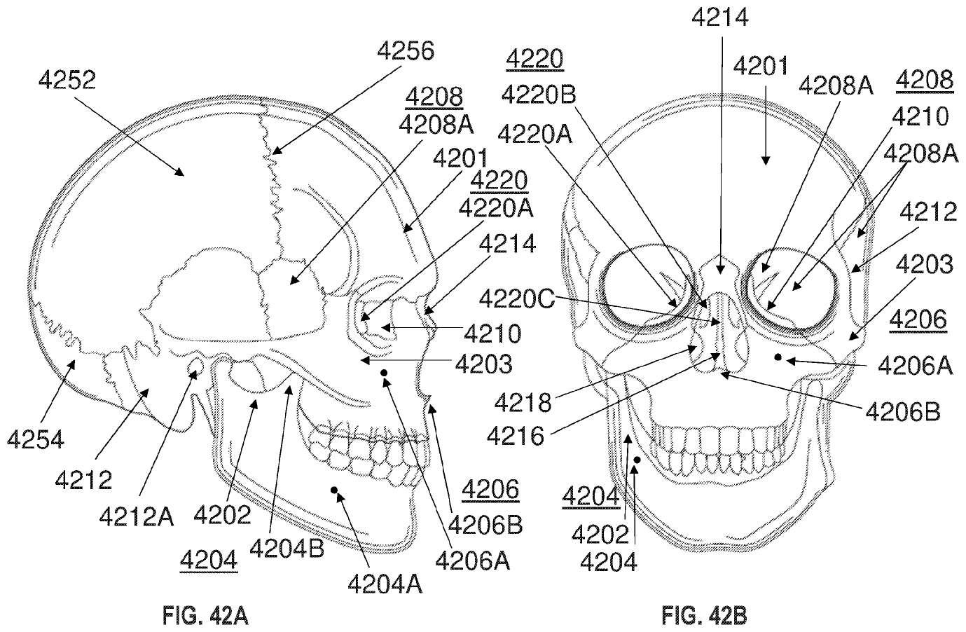

FIGS. 16 and 17 utilize a lower jaw line support coupled to the helmet for distributing force along the jawbone (see FIG. 41). The use of the facepiece alone, with padding or tiles in any of the variations are adapted and configured to distribute force along the bones of the skull and face as well as the jaw (See FIGS. 41, 42A and 42B). According to certain embodiments, lower jaw support may be part of a facepiece.

FIG. 16 is a front view of a head covering (helmet/shell) for inducing vasocompression in the blood supply of a patient receiving chemotherapy 1600. A partial submandibular rigid support or partial mandible frame 1602 extends continuously from a left portion of the mandible body, the mental protuberance or chin, and a right portion of the mandible body. An oversized cushion or full mandible padding 1604 is shown positioned between the patient's jaw and the rigid support 1602. The rigid support 1602 is attached to the helmet/shell 1610 above the eyebrows and two front attachment points or strap adjuster and connector 1606A on a front strap support frame 1606 via straps 1608. The rigid support 1602 is attached to the shell 1610 at the left and right sides from attachment points or strap connector on mandible frame 1602A at the left and right ends of the support 1602. There is also a rear strap support frame 1612 which may also have attachment points for straps 1608.

FIG. 17 is a front view of a head covering (shell) for inducing vasocompression in the blood supply of a patient receiving chemotherapy 1700. Similar to FIG. 16, there is a submandibular rigid support 1702 attached to the shell 1710. Submandibular rigid support 1702 may be a rigid chin support structure in a full mandible support frame. In contrast to FIG. 16, the support 1702 extends continuously from a left portion of the mandible body at the left side angle of the mandible, along the mental protuberance or chin, and a right portion of the mandible body to the right-side angle of the mandible. A cushion 1704 of similar size to the rigid support is positioned between the patient's jaw and the rigid support, which may be a soft foam filler along full mandible support frame 1702. The rigid support 1702 is attached to the shell 1710 above the eyebrows and two front attachment points adjacent the chin such as strap connectors on mandible frame 1702A. The rigid support 1702 is attached to the shell 1710 at the left and right sides from attachment points at the left and right ends of the support such as strap adjuster and connector 1706A on front strap support frame 1706. There is also a rear strap support frame 1712. Straps 1708 connect rigid support 1702 to shell 1710 via front strap support frame 1706 on shell 1710 via strap connectors on mandible frame 1702A and strap adjuster and connector 1706A, as well as attachment points on rear strap support frame 1712.

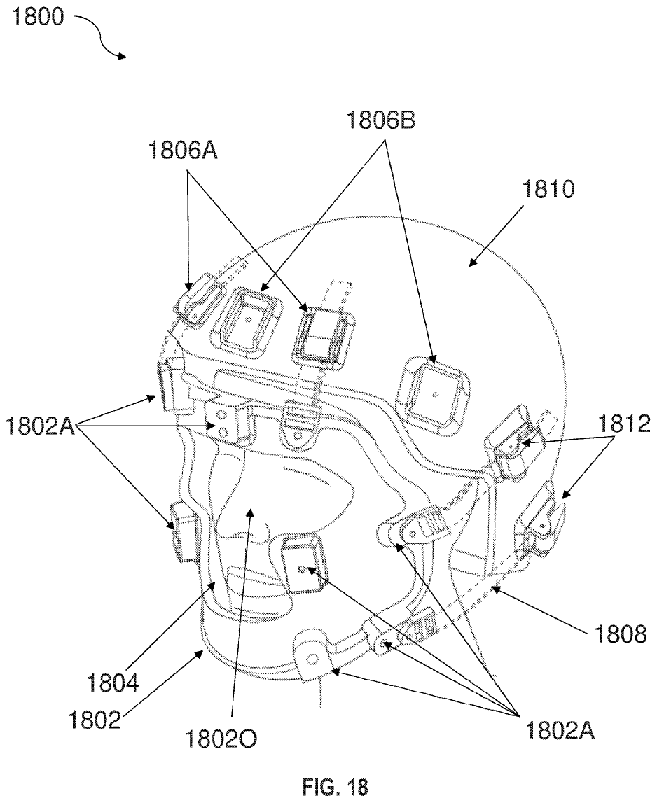

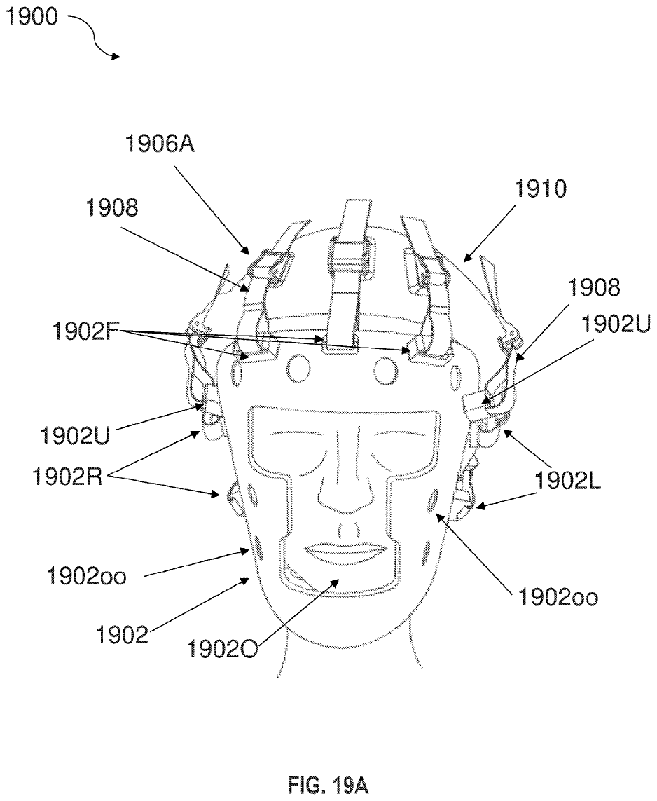

FIG. 18 is a front view of a head covering for inducing vasocompression in the blood supply of a patient receiving chemotherapy that includes a full-face support structure 1800. The full-face support structure 1802 encircles the mandible body and Ramus, extends across and at least partially covering the Zygomatic, Maxilla and Frontal bones of the skull. There is an opening 1802O in the full-face support structure 1802 for the eyes, nose and mouth. A cushion 1804 of similar size and shape to the full-face support structure 1802 is positioned between the patient and the full-face support 1802. The full-face support 1802 is attached to the shell 1810 above the eyes and two top front attachment points or front strap support connector 1806A. The rigid support 1802 is attached to the shell 1810 at the left and right sides from attachment points at the left and right ends of the support 1802 along the mandible body and a location adjacent to a superior aspect of the Ramus, such as strap connectors on face support 1802A. The full-face support 1802 has additional unused attachment points 1802B such as in the middle of the forehead, left and right sides adjacent to the nose and left and right sides adjacent to the chin. One or more of the additional attachment points may be used to further adjust the distribution of forces generated during vasocompression, by connecting to additional unused strap support connector sockets 1806B. There is also a side and rear strap support connector 1812 at the side and rear of shell 1810. Straps 1808 connect full-face support structure 1802 to shell 1810 via front strap support connector 1806A on shell 1810 via strap connectors on face support 1802A, as well as attachment points on side and rear strap support connector 1812.

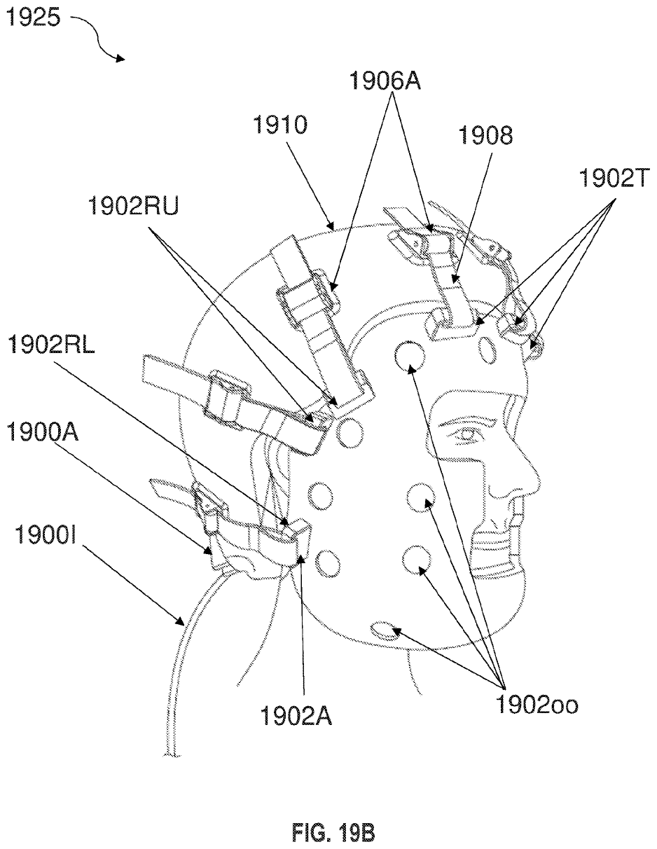

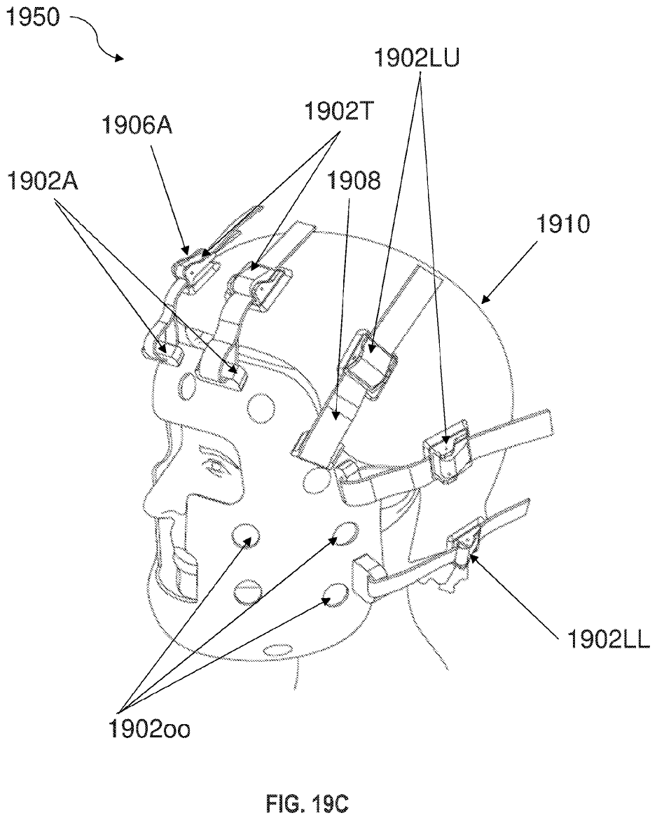

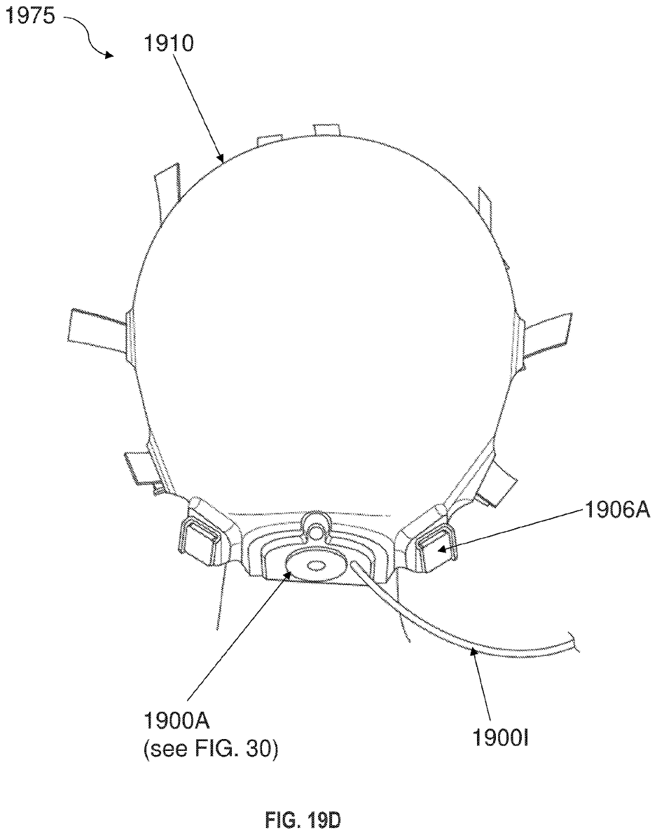

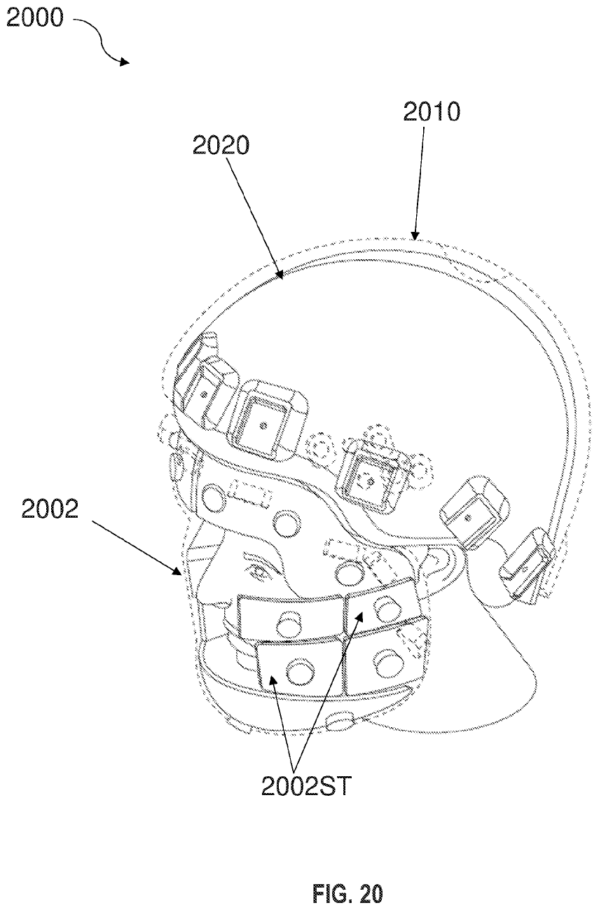

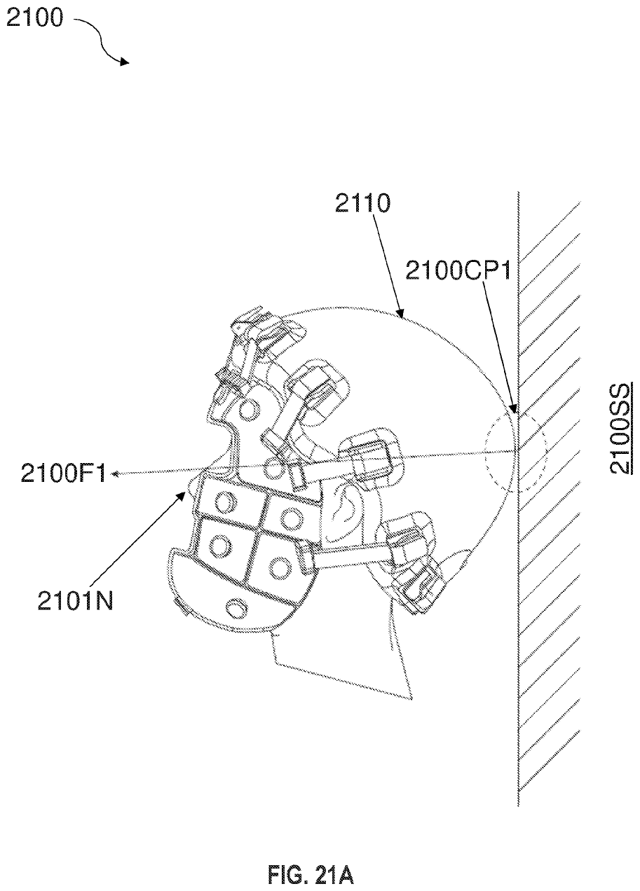

FIG. 19A is a front view of a head covering for inducing vasocompression in the in the blood supply of a patient receiving chemotherapy that includes a full-face support structure 1900. The full-face support structure 1902 encircles the mandible body and Ramus, extends across and at least partially covering the Zygomatic, Maxilla and Frontal bones of the skull. There is an opening 1902O in the full-face support structure for the eyes, nose and mouth. A series of sockets or openings 1902oo in the full-face support structure permit the use of an array of independently selectable cushion tiles or other localized support structures to be added to the full-face support structure 1902. FIGS. 20 to 26 illustrate various configurations of the cushion tiles adapted for use with the full-face support. The cushion tiles are not shown in the various views of FIGS. 19A, 19B and 19C. According to certain embodiments, the full-face support 1902 is attached to the shell 1910 at nine points, three each along the forehead 1902F, the right side 1902R and the left side 1902L. Along the forehead is one each above each eye and one in the middle of the forehead above the nose. A pair of upper attachment points 1902U are located near the temples. Left and right lower attachment points are located adjacent to the Ramus below the ear. Each of the attachment points on face support 1902F/1902U/1902R/1902L are D-shaped connectors sized to receive a loop that is part of a strap 1908 attached to an adjustable connection point/strap support connector 1906A on the shell 1910. Each one of the straps 1908 and associated connectors 1902F/1902U/1902R/1902L/1906A may be adjusted to fit the patient and while also increasing comfort during use by the distribution of forces generated during vasocompression.

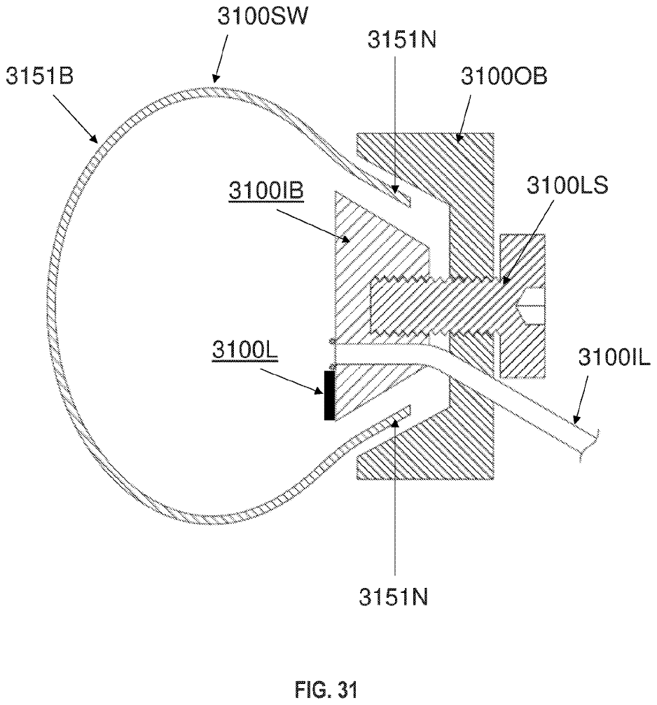

FIG. 19B is a right-side view of a head covering for inducing vasocompression as shown in FIG. 19A 1925. In this view, the top three attachment points 1902T along with the pair of right upper attachment points 1902RU and the right lower attachment point 1902RL are visible. There is also inflation line 1925I and inflation adapter 1925A (see FIG. 31) which connect to shell 1910. Also shown are sockets to place localized support structures/strap connector on face support 1902oo, strap connector on face support 1902A, strap 1908, and strap support connector 1906A on the shell 1910.

FIG. 19C is a left-side view of a head covering for inducing vasocompression as shown in FIG. 19A 1950. In this view, two of the top three attachment points 1902T along with the pair of left upper attachment points 1902LU and the left lower attachment point 1902LL are visible. Also shown are sockets to place localized support structures/strap connector on face support 1902oo, strap connector on face support 1902A, strap 1908, and strap support connector 1906A on the shell 1910.

FIG. 19D is a rear view of a head covering for inducing vasocompression as shown in FIG. 19A 1975. In this view, a number of the attachment points on the shell 1910 are shown. Additionally, the pneumatic connection (inflation adapter) 1900A and inflation line 1900I in the back rear of the shell 1910 are visible. Also visible is strap support connector 1906A on the shell 1910.

According to certain embodiments, headband 1900H may be connected to head covering/shell/helmet 1910 via bands, straps 1908A, or other structures in an adjustable gap between helmet 1910 and face support 1902 that is adjusted by straps 1908. Headband 1900H may stabilize helmet 1910 during or before/after inflation of helmet 1910 via inflation line 1900I which may be coupled to an inflation source. In some embodiments, the patient may don the headband 1900H before wearing the helmet 1910, followed by face support 1902.

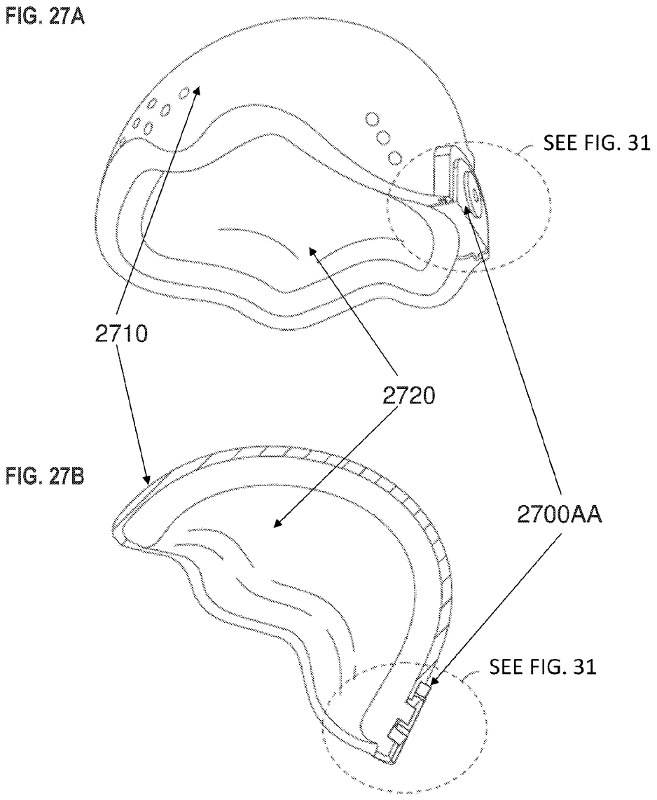

FIG. 20 is a top isometric view of an embodiment of a multiple tile face cushion adapted for use with the embodiments of FIGS. 19A-19D 2000. The full-face shield and the straps and connectors are omitted from this view. As shown here, there is helmet 2010 which is transparent to show location of inflatable structure 2020 (see also FIGS. 27A, 27B, 29A, 29B, 29C, 30A, 30B, 31). Also shown is rigid face support structure 2002, which is transparent to show location of tile elements/support tiles 2002ST. Each of the tiles 2002ST has a plug which is sized and positioned to align with and be received by the corresponding opening in the face shield. To achieve load spreading, the cushion properties for each face shield may be adapted by selecting cushion tiles 2002ST of desired shape, size, composition along with the placement of specific tiles in the location and orientation in the face shield to improve comfort for the patient experiencing vasocompression therapy. According to certain embodiments, and to further achieve load spreading, the tiles 2002ST may have different rigidities, durometer, flexibilities, contours, and other characteristics based on position on face support 2002 and adjacent bony anatomy and patient comfort (see FIGS. 41 and 42A-B).

FIGS. 21A-D represent the variation of force on the helmet to produce a counterforce acting through the user's head and face to provide some relief to discomfort experienced during treatment.

FIG. 21A is a side view of a vasocompression device of FIGS. 19A-19D with the face shield removed showing the position of an exemplary multiple tile face cushion 2100. In this view, the patient is in a prone position with the shell 2110 positioned against a support structure 2100SS which produces a counter force vector indicated by the arrow 2100F1. The counter force vector 2100F1 is used to provide additional comfort to the patient undergoing vasocompression therapy by offsetting a portion of the forces generated during the application of vasocompression therapy. In this embodiment, the counter force 2100F1 is directed from the contact point 2100CP1 on the support structure 2100SS towards the upper attachment point and the nose of the patient 2101N.

FIG. 21B is a side view of a vasocompression device of FIG. 21A with the face shield removed showing the position of an exemplary multiple tile face cushion 2125. In this view, the patient is in a prone position with the shell 2110 positioned against a support structure 2100SS which produces a counter force vector 2100F2 indicated by the arrow. The force vector 2100F2 is used to provide additional comfort to the patient undergoing vasocompression therapy by offsetting a portion of the forces generated during the application of vasocompression therapy. In this embodiment, the counter force 2100F2 is directed from the contact point 2100CP2 on the support structure 2100SS towards the upper attachment point, past the ear and below the chin of the patient 2101C.

FIG. 21C is a side view of a vasocompression device of FIG. 21A with the face shield removed showing the position of an exemplary multiple tile face cushion 2150. In this view, the patient is in a prone position with the shell 2110 positioned against a support structure 2100SS which produces a force vector indicated by the arrow 2100F3. The force vector 2100F3 is used to provide additional comfort to the patient undergoing vasocompression therapy by offsetting a portion of the forces generated during the application of vasocompression therapy. In this embodiment, the counter force 2100F3 is directed from the contact point 2100CP3 on the support structure towards the upper attachment point, past the ear and above the chin of the patient 2101C.

FIG. 21D is a side view of a vasocompression device of FIG. 21A with the face shield removed showing the position of an exemplary multiple tile face cushion 2175. In this view, the patient is in an upright position and the external force is imparted to the shell 2110 which produces a force vector indicated by the arrow 2100F4. The force vector 2100F4 is used to provide additional comfort to the patient undergoing vasocompression therapy by offsetting a portion of the forces generated during the application of vasocompression therapy. In this embodiment, the counter force 2100F4 is directed from the contact point 2100CP4 towards the upper attachment point, above the ear and towards a line between the nose 2101N and the chin 2101C of the patient.

FIGS. 22-25 illustrate a number of different support structures to aid in force distribution by changing contact area or location of addition of weighted structures to provide comfort during treatment.

FIG. 22 is a side view of a vasocompression device of FIG. 21A with the face shield removed showing the position of an exemplary multiple tile face cushion 2200. There is a support ring 2200R shown in cross section at a point on the shell 2210 central to the counter force vector 2100F4 shown in FIG. 21D. In use, the support ring 2200R would be placed in contact with a support structure or other fixture used to impart the counter force, such as support structure 2100SS shown in FIG. 21C. The ring 2200R would be used to distribute the force across a larger area of the head covering.

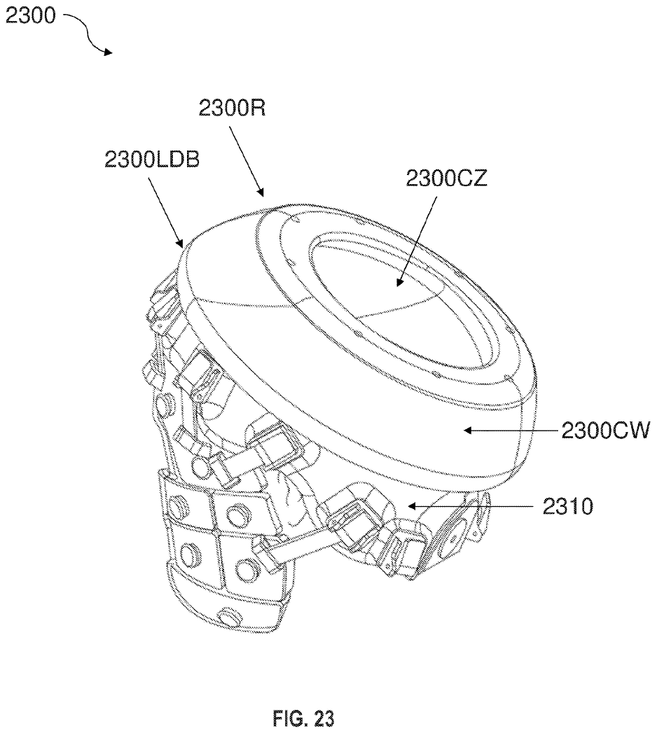

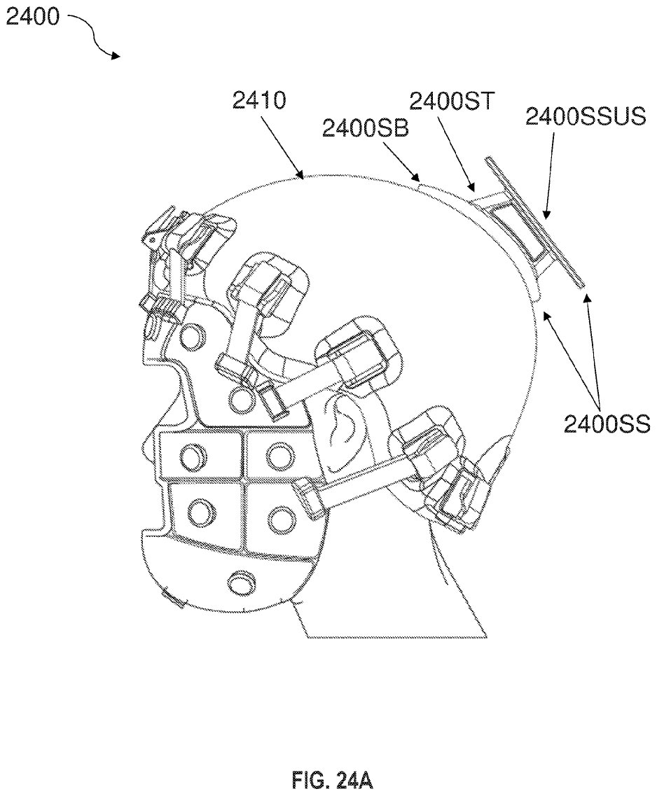

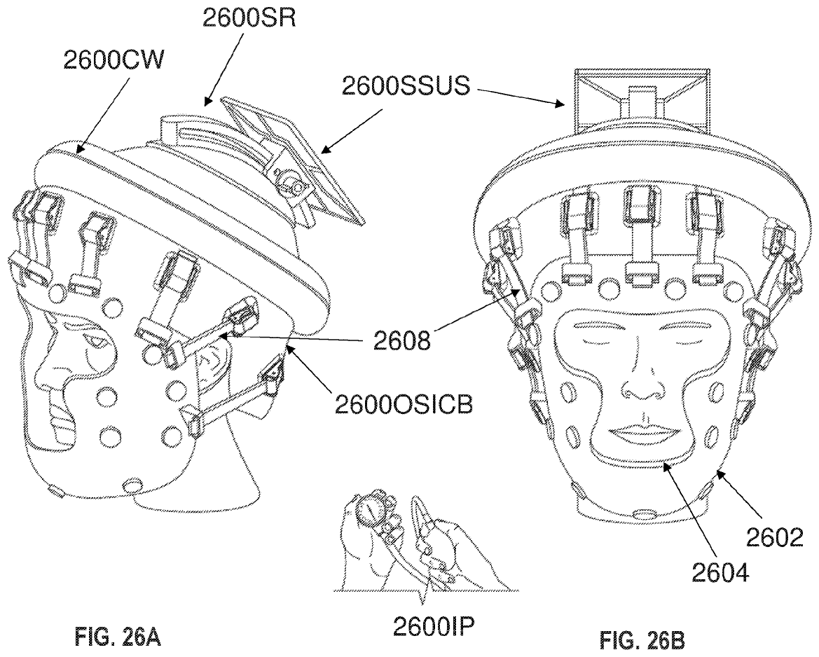

FIG. 23 is a rear perspective view of a vasocompression device of FIG. 21A with the face shield removed showing the position of an exemplary multiple tile face cushion 2300. There is a support ring 2300R shown in position on the head covering 2310. The support ring 2300R has a frustoconical shape with a larger diameter base 2300LDB against the surface of the shell 2310. The flat upper ring surface may be used to receive and distribute a counter force such as a counterweight in rim 2300CW. Optionally, the upper support ring 2300R may be used as a contact zone/second support base 2300CZ for other or additional support structures such as in FIG. 26.

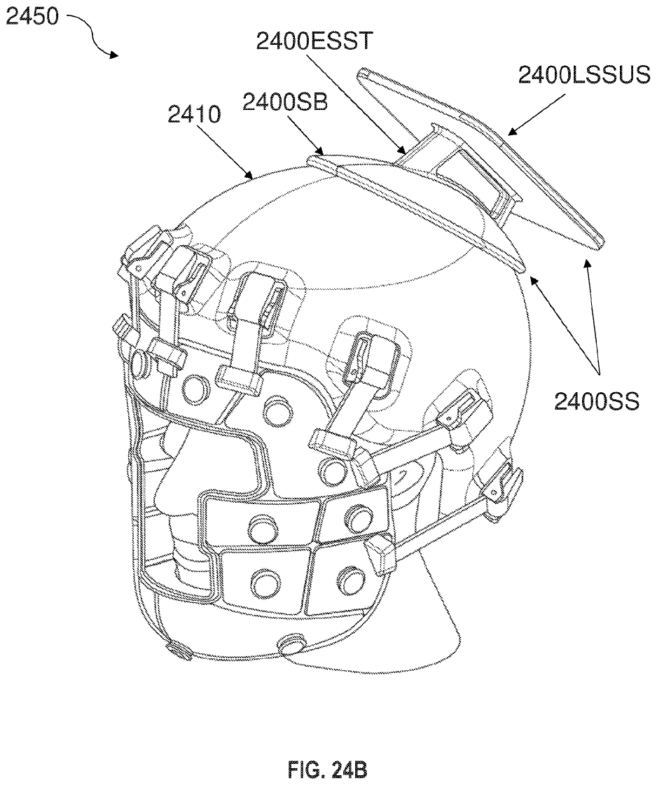

FIG. 24A is a side view of a vasocompression device of FIG. 21A with the face shield removed showing the position of an exemplary multiple tile face cushion 2400. There is a support structure 2400SS shown in position on the head covering 2410. The support structure 2400SS has a base 2400SB, a stem 2400SST and an upper support surface 2400SSUS. The base 2400SB may be sized to distribute the counter force across a larger portion of the shell 2410 while the direction and contour of the stem 2400SST and upper support surface 2400SSUS may be configured to engage with a corresponding support surface.

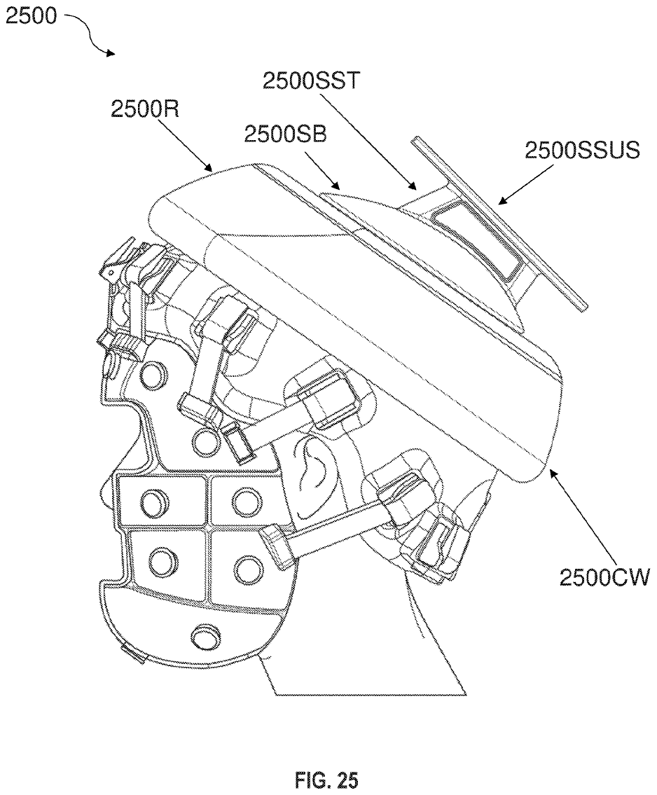

FIG. 24B is a front perspective view of the embodiment of FIG. 24A 2450. Similar to FIG. 24A, FIG. 25B illustrates a support structure 2400SS shown in position on the upper rear of the head covering 2410, approximately near the crown of the head of the patient 2401 wearing the head covering 2410. Like the embodiment in FIG. 24A, the support structure has a base 2400SB. In contrast to the embodiment of FIG. 24A, the embodiment shown in FIG. 25B includes an elongated stem 2400ESST and a relatively larger upper support surface 2400LSSUS. The base 2400SB may be sized to distribute the counter force across a larger portion of the shell 2410 while the direction and contour of the elongated stem 2400ESST and upper support surface 2400LSSUS may be configured to engage with a corresponding support surface. According to certain embodiments, upper support surface 2400LSSUS may have an anti-slip surface or grip treatment for improved contact and stability with a corresponding support surface.

FIG. 25 is a side view of a vasocompression device of FIG. 21A with the face shield removed showing the position of an exemplary multiple tile face cushion 2500. The support ring 2500R shown in position on the head covering is similar to the embodiment of FIG. 23. The support ring 2500R has a frustoconical shape with a larger diameter base 2500SB against the surface of the shell 2510. Base 2500SB may be contoured to mate with upper surface of counterweight rim/annulus 2500CW or 2300CW on shell 2310 (see FIG. 23). The flat upper ring surface 2500SSUS may be used to receive and distribute a counter force, and is supported by helmet support/stem 2500SST. In this view, the upper support ring 2500R is used as a support base 2500SB for the support structure illustrated in FIGS. 25A and 25B. The combination advantageously allows for a wider distribution of the counter force while permitting better accommodation with the support structure because of the elongate stem and support surface.

FIGS. 26A and 26B provide a range of variations to adjust the fit and provide comfort. One or more of the following adjustable or user-determined aspects may be used to used alone or in a number of combinations to provide comfort or adjust the application of forces used to induce vasocompression: the variable position platform on the top of the helmet, the pressure within the balloon liner/bladder, individual straps around the perimeter of the facepiece, as well as user/patient customized and adjustable tiles within the facepiece and overall shape and size of the facepiece.

FIG. 26A is a left side view of an embodiment of a vasocompression device having a stabilization weight 2600CW and a head support/head rest 2600SSUS on an adjustable, sliding mount 2600SR which may be used to adjust head rest angle. As shown here, there is also an outer shell with internal compression balloon 2600OSICB, which may be inflated via an inflation bulb/pump 2600IP to balloon and adjustment strap(s) 2608.

FIG. 26B is a front view of the vasocompression device in FIG. 26A. Shown here are adjustment straps 2608, facial support 2602, and facial padding 2604.

FIG. 27A is a bottom-up perspective view of an interior of a head covering showing the helmet/shell 2710 and inflatable balloon liner 2720. The air adaptor 2700AA and connection for adjusting the pressure within the balloon liner 2720 is also shown at the rear of the shell 2710 (see also FIG. 31).

FIG. 27B is a cross section view of the head covering of FIG. 27A showing the arrangement of the shell 2710, the liner 2720 and air adaptor 2700AA and connections.

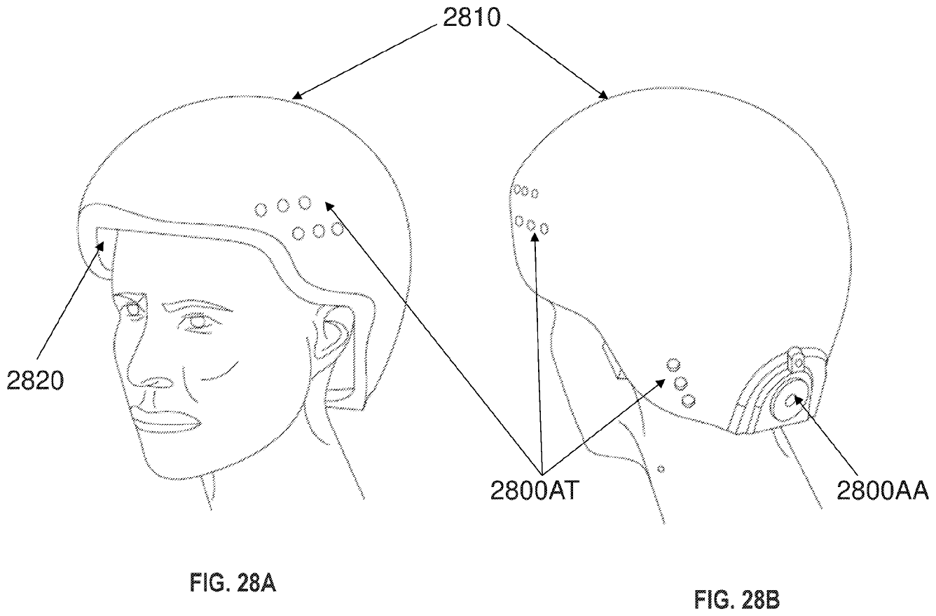

FIGS. 28A-28B are front and rear views of an embodiment of the head covering of FIGS. 27A-27B shown in position on a head. A series of attachment points 2800AT for various accessories, connectors, and supports are shown extending rearward from the upper corners of helmet 2810 in the view of FIG. 28A and along the rear surface as shown in FIG. 28B. Also shown is balloon liner 2820 in FIG. 28A and air adaptor 2800AA in FIG. 28B.

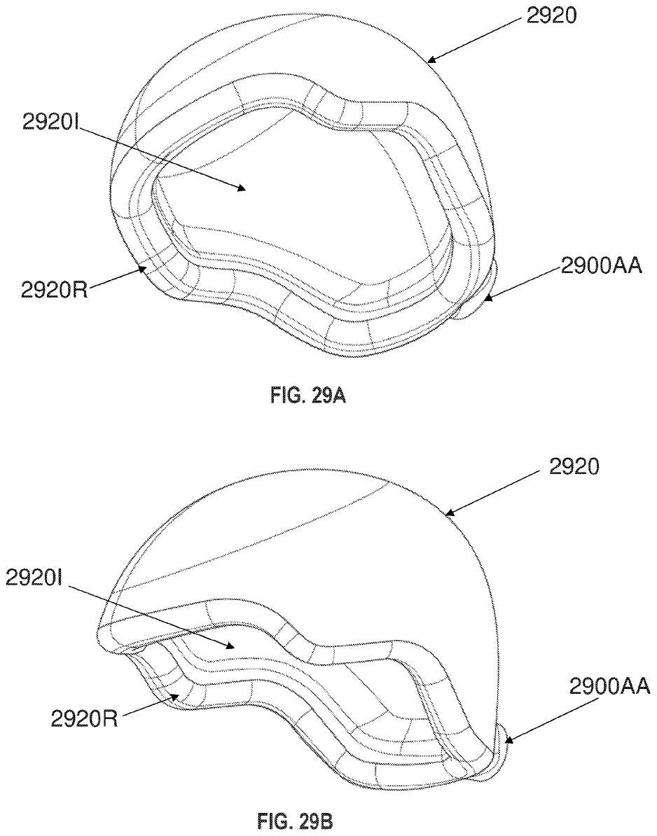

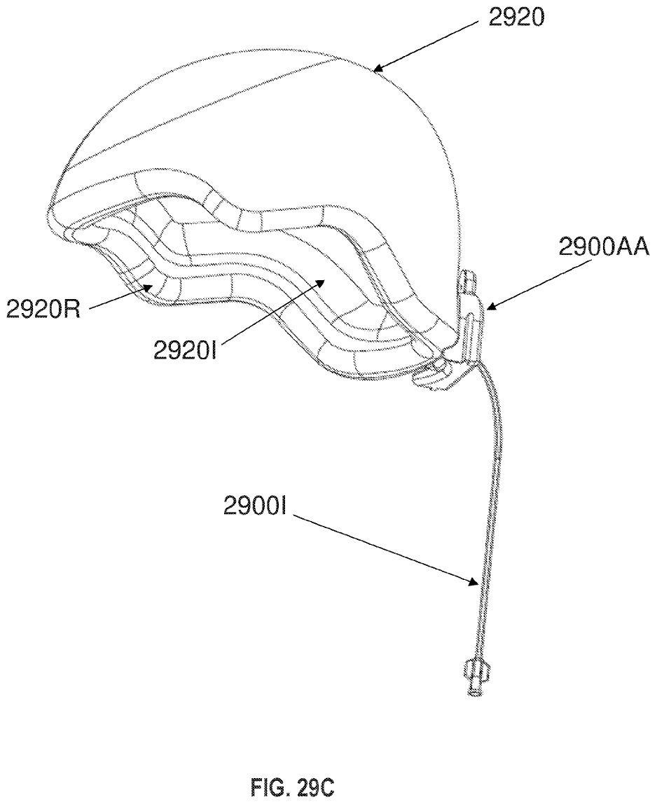

FIGS. 29A-29B is an inferior perspective and left perspective view of a balloon liner, respectively. As shown here there is a balloon liner 2920 having a rim 2920R and interior 2920I. Also shown is air adaptor 2900AA.

FIG. 29C is a left perspective view of a balloon liner. As shown here there is balloon liner 2920, rim 2920R, and interior 2920I. An inflation/air adaptor 2900AA and connection such as inflation line 2900I for adjusting the pressure within the balloon liner 2920 is also shown at the rear of the balloon liner 2920.

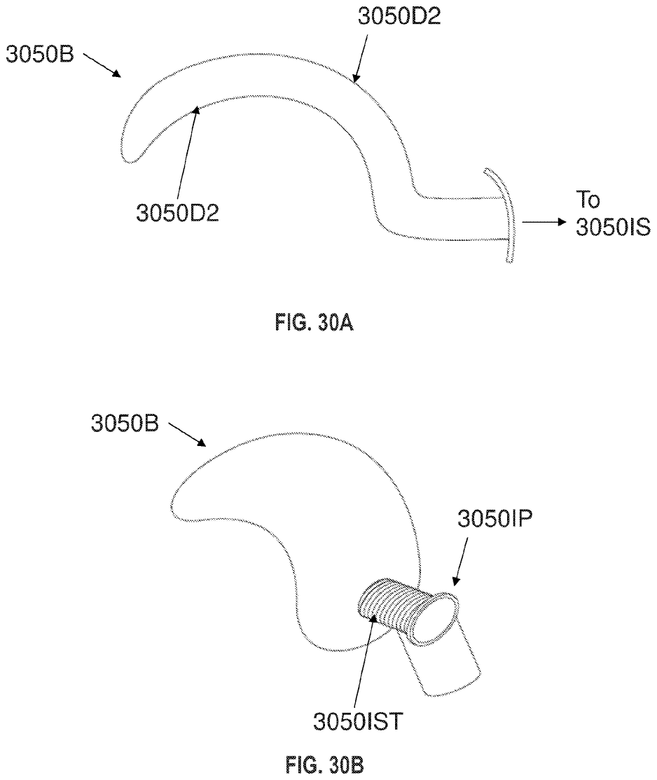

FIG. 30A is a cross section of an inflatable bladder. In one aspect, the inflatable bladder 3050B is formed from an elastomer having a first durometer 3050D1 adjacent to the inner wall of the head covering and a second durometer 3050D2 adjacent to the hair/scalp of the patient. The bladder material may be selected from Latex, Silicone, Polyurethane, or any other flexible polymer. In some embodiments, the second durometer is lower than the first durometer. In still other embodiments, the durometer range is from 5-55 Shore A. Additionally, or optionally, the thickness of the inflatable bladder 3050B adjacent to the inner wall of the head covering is greater than the thickness of the inflatable bladder 3050B adjacent to the patient's scalp. In one embodiment, the thickness of the inflatable bladder 3050B adjacent to the patient's scalp is from 20-80 microns. In still other embodiments, the inflatable bladder 3050B is a one-piece bladder with various wall thicknesses, for example, from 30-100 micrometer wall thickness at the scalp side and then from 100-300 micrometer wall thickness at any other sides. According to certain embodiments, inflatable bladder 3050B is connected to an inflation source 3050IS.

FIG. 30B is a is a rear perspective view of the inflatable bladder in FIG. 30A. As shown here, there is an outer surface of inflation bladder 3050B coupled to an inflation stem 3050IST and inflation port 3050IP.

FIG. 31 is a cross section view of an inflatable bladder joint to a helmet with an inflation port. As shown here, there is a side wall 3100SW of an interior of inflation bladder 3151B. Further shown is an inner block 3100IB, liner 3100L on inner block 3100IB to engage bladder side wall 3100SW. There is also inflation line 3100IL, and outer block 3100OB to engage with inner block 3100I3 via locking screw 3100LS which may also anchor a neck 3151N of inflatable bladder 3151B for passage of inflation line 3100IL.

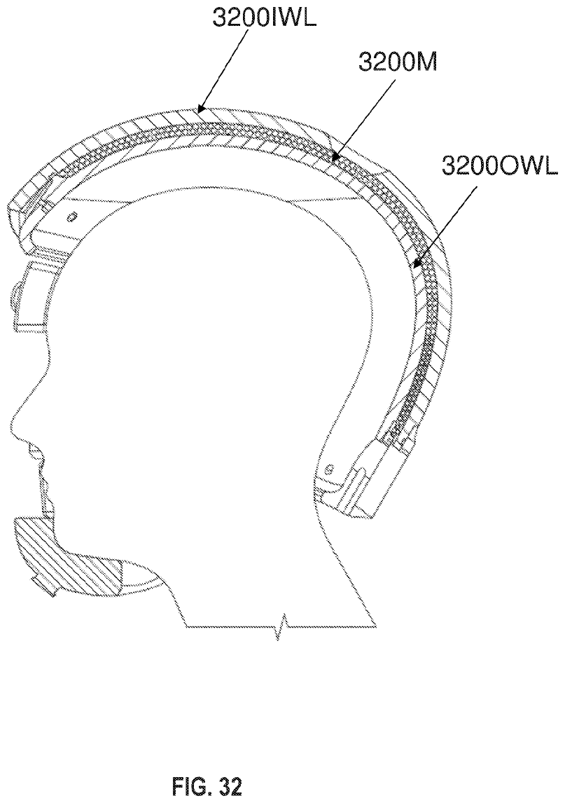

FIG. 32 is a cross section view of a head covering for inducing vasocompression embodiment similar to FIG. 19C having the tile cushions in place. The detailed section view of the shell shows that there is a secondary layer of material between the outer wall of the inflatable liner 3200OWL and the inner wall of the shell 3200IWL. The material 3200M may be any suitable solution, particle suspension, composite material, gel, aerogel or other material suited for force distribution or absorption of the vasocompression force between inner wall of shell 3200IWL and outer wall of inflatable liner 3200OWL.

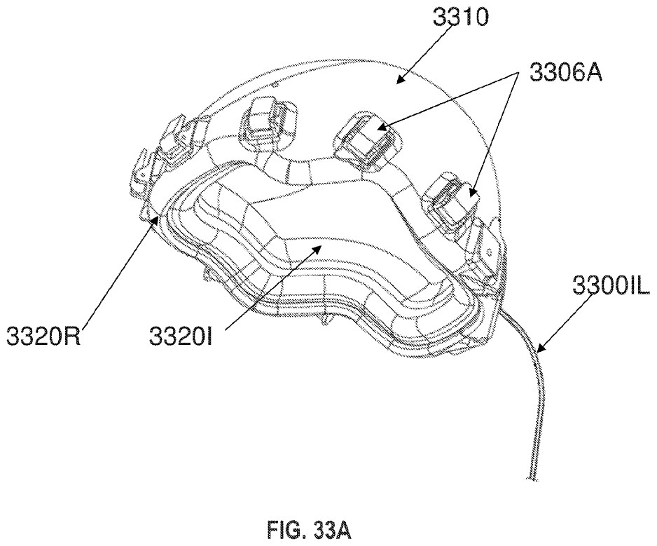

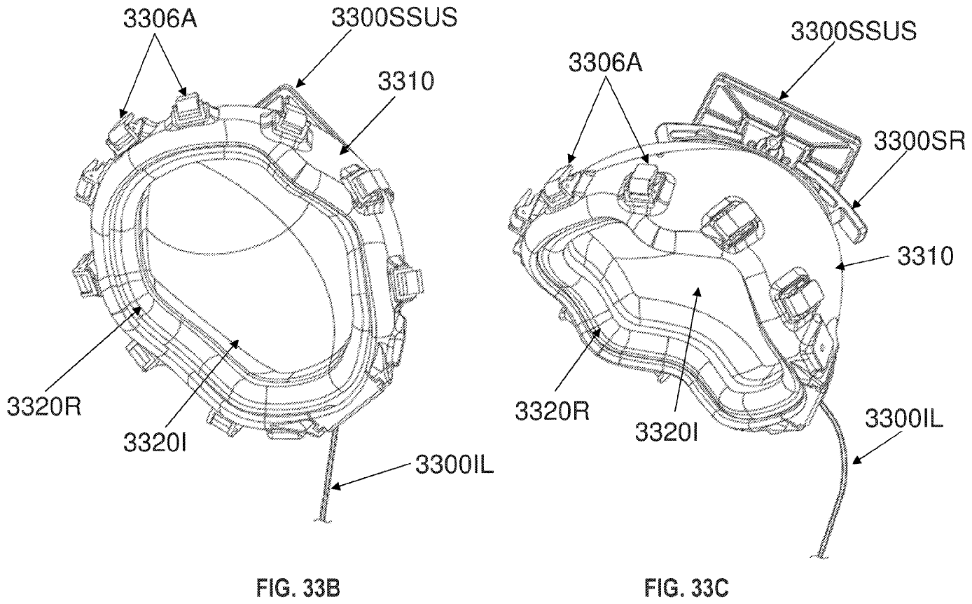

FIG. 33A is a left-side perspective view of the balloon liner inside a helmet. As shown here, there is a rim 3320R and interior 3320I of the balloon liner, helmet 3310 with strap connectors 3306A. Also shown in inflation line 3300IL.

FIGS. 33B-33C is an inferior and left-side perspective view of a balloon liner inside a helmet having a head rest, respectively. As shown here, there is a rim 3320R and interior 3320I of the balloon liner, helmet 3310 with strap connectors 3306A, as well as inflation line 3300I. A head support/head rest 3300SSUS is shown on an adjustable, sliding mount 3300SR, which may be used to adjust head rest angle.

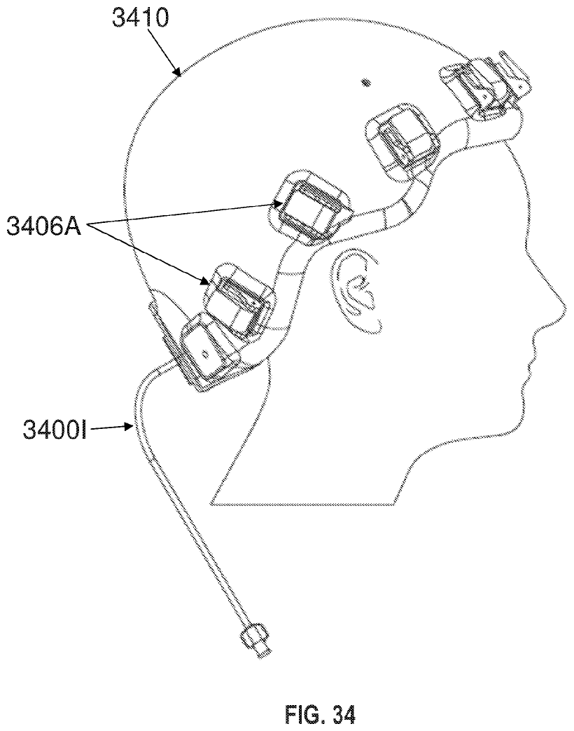

FIG. 34 is a right-side view of an embodiment of the head covering of FIGS. 33A-33C shown in position on a head. Shown here is helmet 3410 with strap connectors 3406A, as well as inflation line 3400I.

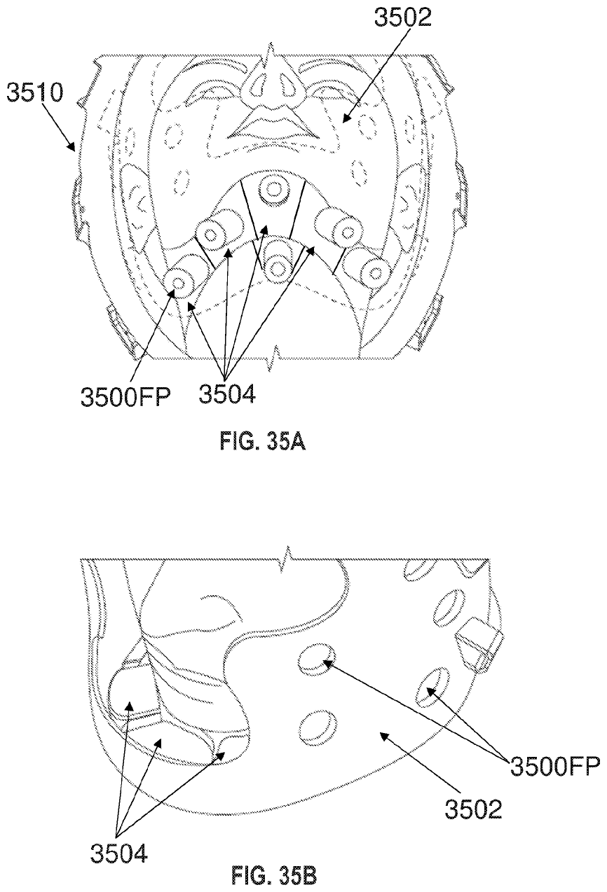

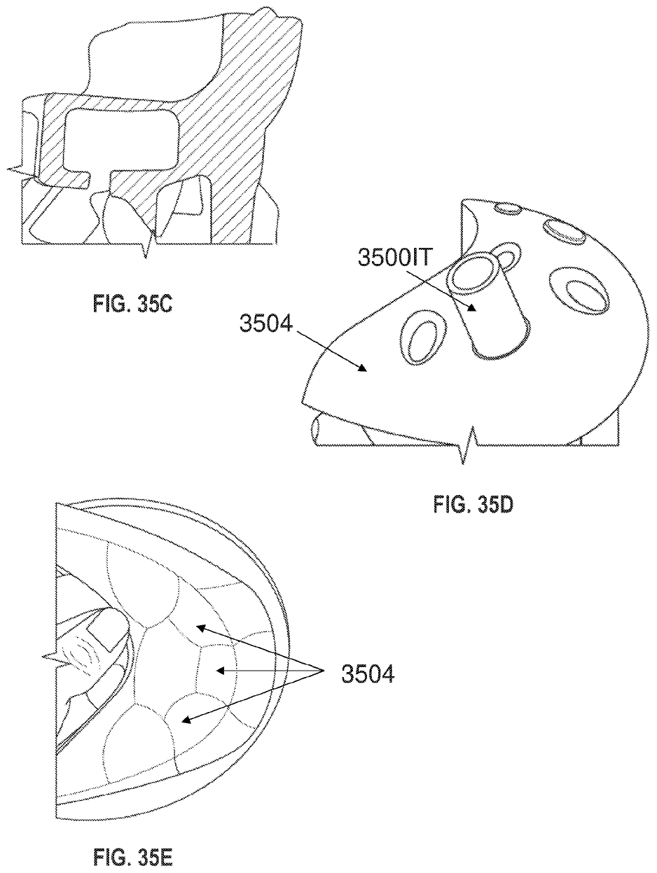

FIGS. 35A-E is a variate of facepiece tile construction using variable density materials or a structure for using air pressure to increase or decrease individual tiles. This may include an all-foam interior or a composite of foam, gel and structure for variable firmness, or air filled, or adjustable air filled via a control valve and air/inflation system or part of an air/inflation system used to control balloon liner pressure, inflation and deflation. According to certain embodiments, tiles may be coupled or contoured to pads, and some or all tiles may be mechanically adjusted or pneumatically adjusted to offset pressure on the bones of the face, and tiles may be divided into various zones of tiles to correspond with various bony structures of the face.

FIG. 35A is a bottom-up view of six air cells around chin. Shown here is helmet 3510, fill ports/connectors 3500FP, and air inflated pads 3504, and a transparent rigid face covering 3502 relative to air pads 3504 and alignment of fill ports 3500FP.

FIG. 35B is a top-down view of the air cells in FIG. 35A. Other air cells are omitted for clarity. Shown here are fill ports/connectors 3500FP inside openings, air inflated pads 3504, and a rigid face covering 3502.

FIG. 35C is a cross section view of an air cell within support structure in FIG. 35A.

FIG. 35D is a side view of the air cells/pads 3504 in FIG. 35A with an exemplary inflation tube 3500IT connected to one of the air cells/pads 3504.

FIGS. 35A and 35D may illustrate tubes, ports, etc. to inflate or deflate individual tiles.

FIG. 35E is a bottom-up view of exemplary size and arrangement of inflated air cells/pads 3504 around the chin.

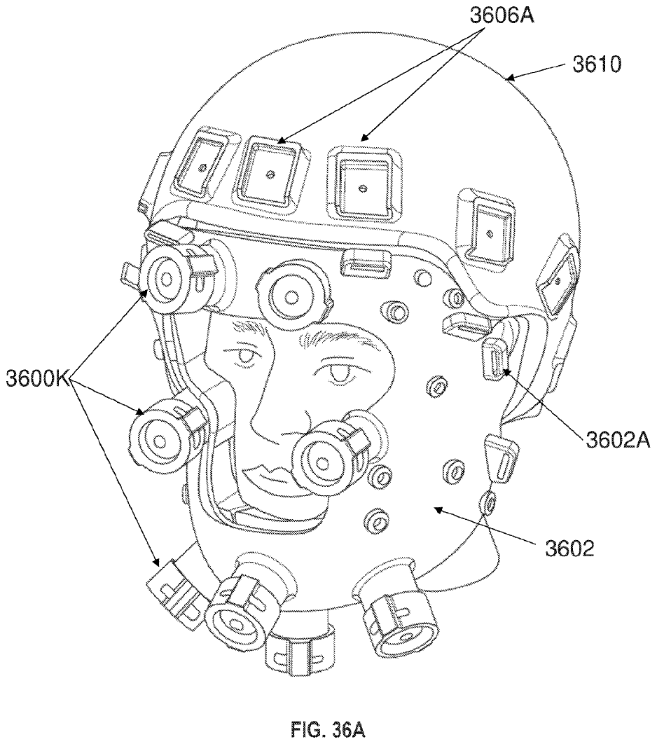

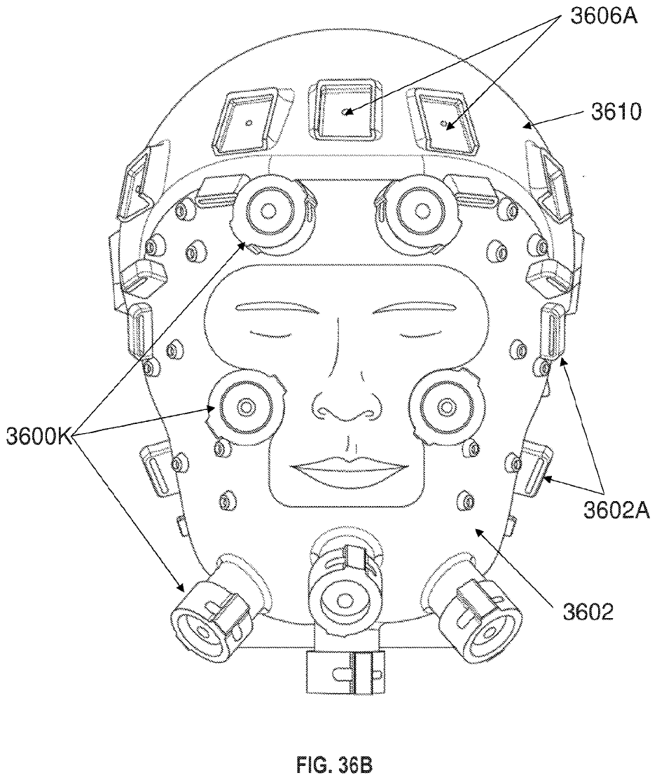

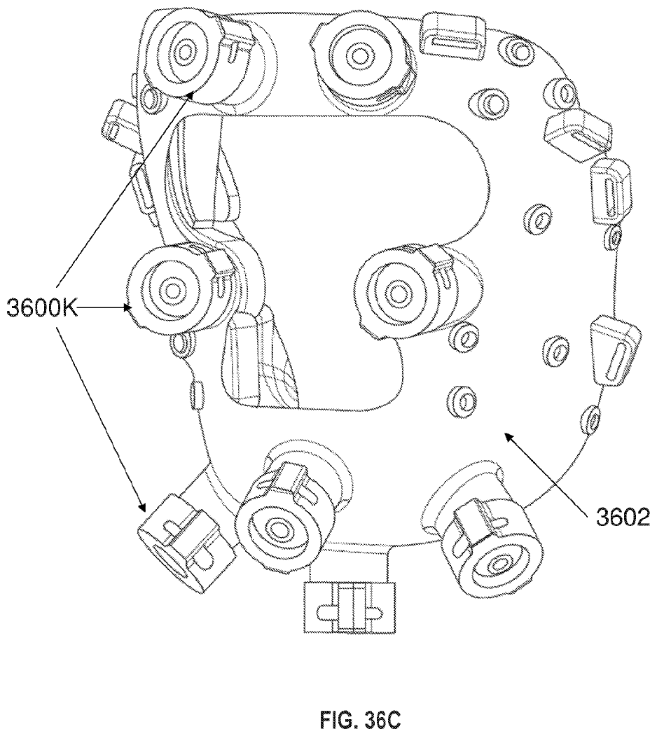

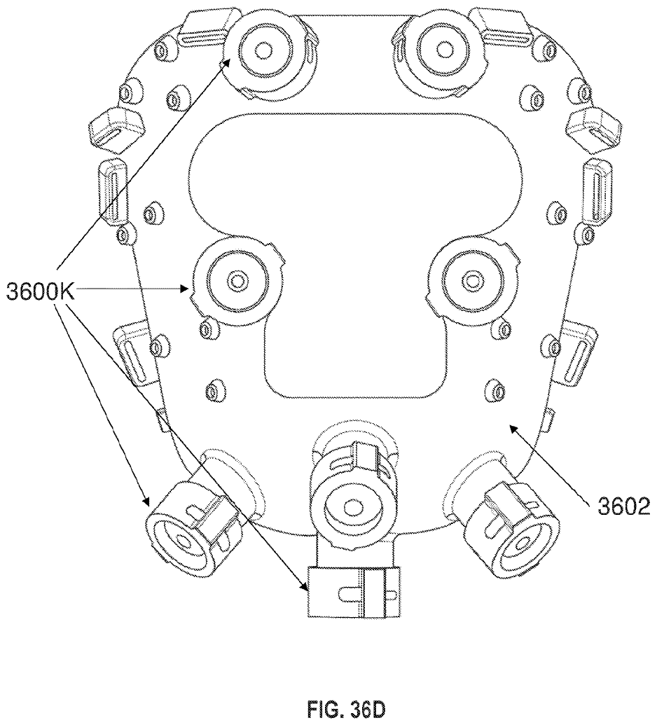

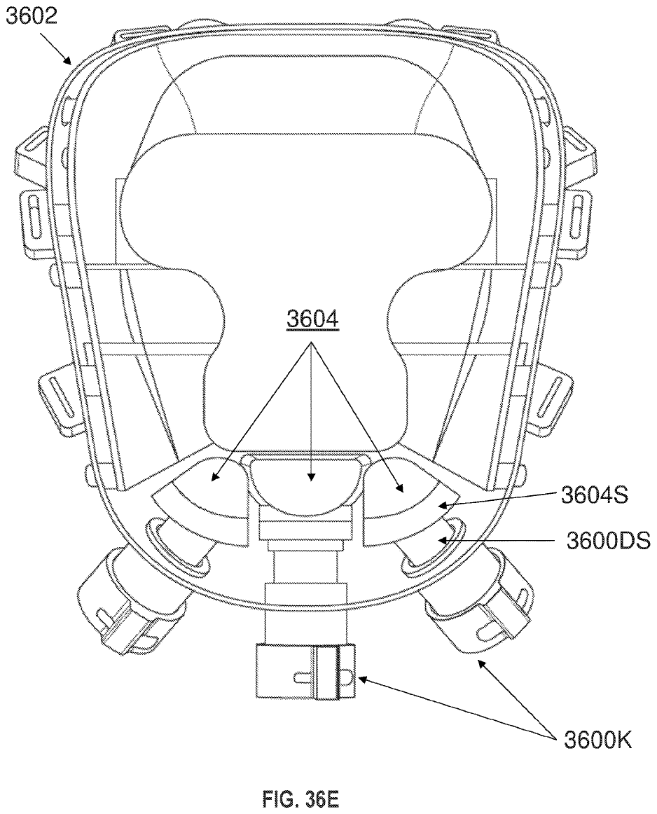

FIG. 36A is a front isometric view of a face shield having eight individual pad adjustment zones with knobs 3600K on the face shield 3602 in relation to the helmet/shell 3610. Straps have been removed for clarity. Also shown is strap connection point 3606A on helmet/shell 3610, strap connection point 3602A on face shield 3602 which according to some embodiments, may be a facepiece support 3602.

FIG. 36B is a front view of the face shield of FIG. 36A showing the same features.

FIG. 36C is a front isometric view of only the face shield of FIG. 36A without the helmet/shell 3610. Shown here are rigid facepiece support 3602 and pad adjustment knob 3600K.

FIG. 36D is a front view of the face shield/facepiece support 3602 of FIG. 36A without the helmet. Patient head and straps have also been removed for clarity.

FIG. 36E is a rear (interior) view of the face shield of FIG. 36D showing the individual pad shapes and positions each one controllable by operation of the dedicated pad adjustment knob 3600K, which may be coupled to a drive shaft 3600DS. Pads 3604 have a pad support 3604S. According to certain embodiments, each pad 3604 or groups of pads 3604 may be sized, contoured, and structured with variable hardness, durometer, and/or stiffness for specific locations on the face and its bony structures.

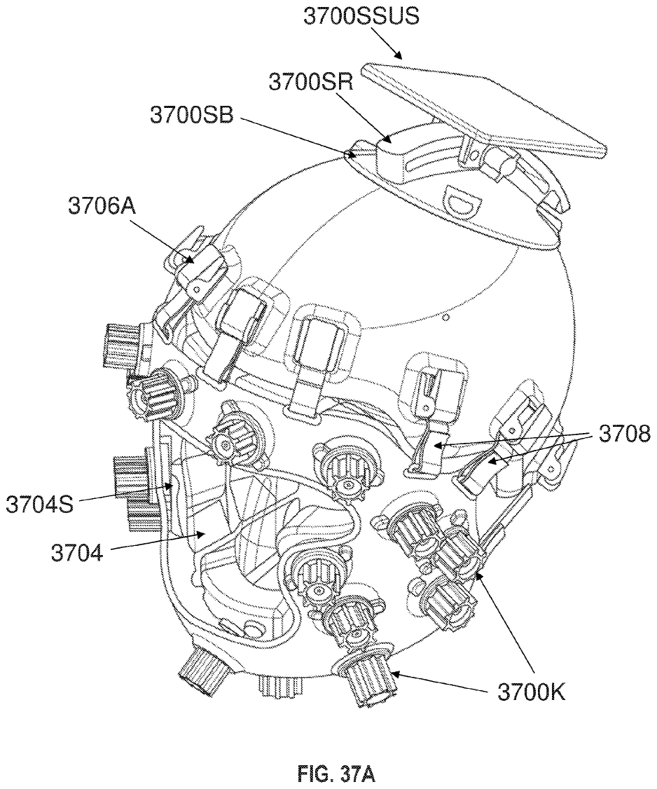

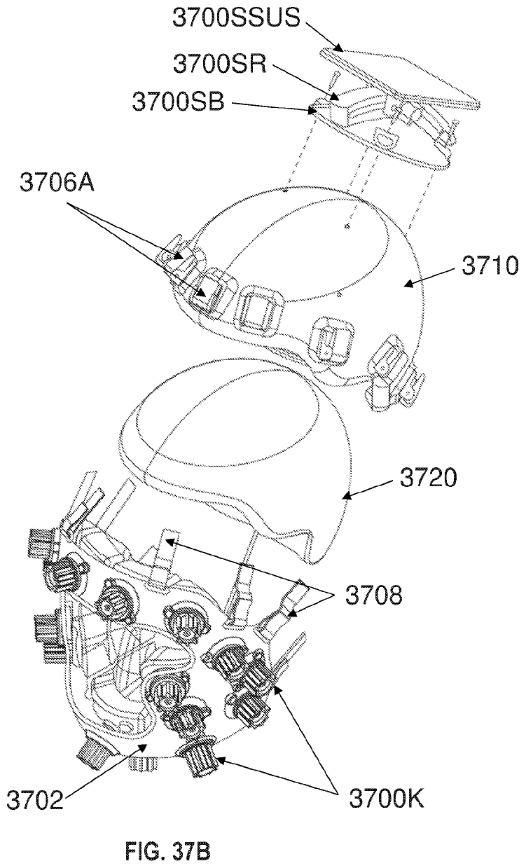

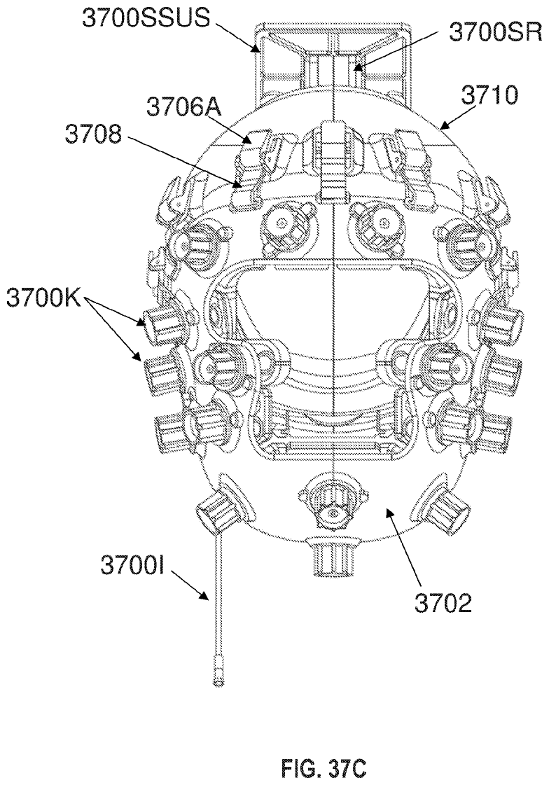

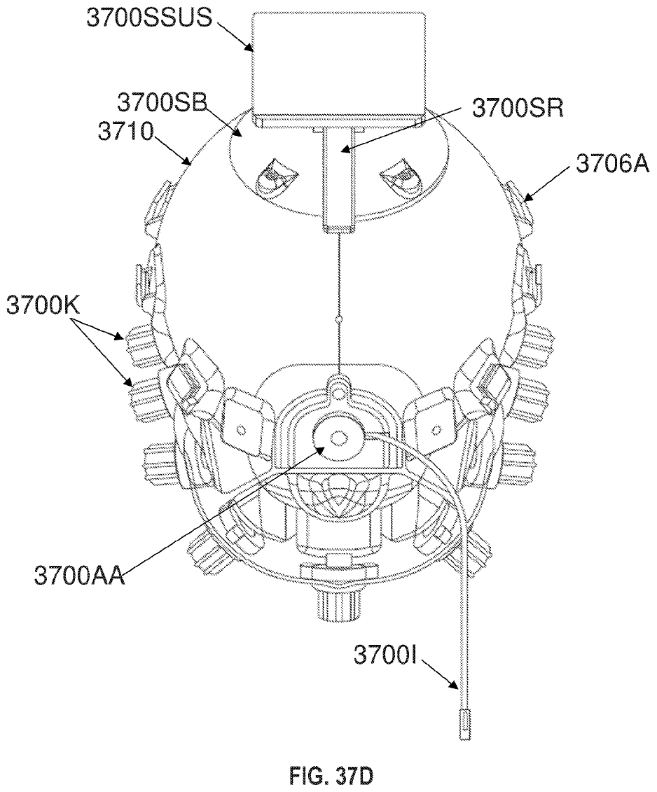

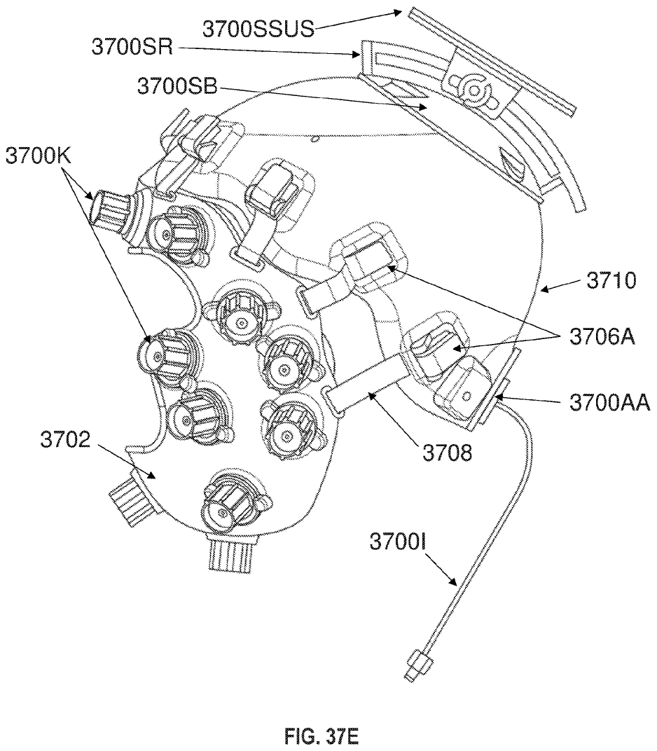

FIG. 37A is an isometric view of a helmet with moveable support and dynamic pressure adjustment facepiece. Facepiece 3702 has pads 3704 with pad support 3704S as well as pad adjustment knob 3700K. Helmet/shell 3710 has a moveable support base 3700SB, a head support/head rest or moveable support 3700SSUS on an adjustable, sliding mount/slide rail for moveable support 3700SR which may be used to adjust head rest angle.

FIG. 37B is an exploded view of the vasocompression device of FIG. 37A. As shown here, there are pad adjustment knobs on 3700K on dynamic facepiece 3702, straps 3708, helmet/shell 3710 with helmet strap clips 3706A. Shown again is helmet/shell 3710 having a head support/head rest or moveable support 3700SSUS on an adjustable, sliding mount/slide rail for moveable support 3700SR which may be used to adjust head rest angle. While illustrated with the sliding support structure of FIGS. 26A and 26B, it is to be appreciated that the helmet/shell 3710 may include various mounts so that a range of different support structures (see FIGS. 22-25) or other additional attachment or connection points may be interchangeably used.

FIG. 37C is a front view of the vasocompression device of FIG. 37A. Vasocompression device has a full helmet/shell 3710 with moveable support 3700SB and dynamic facepiece 3702. As shown here, there are pad adjustment knobs on 3700K on dynamic facepiece 3702, straps 3708, helmet/shell 3710 with helmet strap clips 3706A. Also shown again is a moveable support 3700SSUS on an adjustable, sliding mount/slide rail for moveable support 3700SR which may be used to adjust head rest angle.

FIG. 37D is a rear view of the vasocompression device of FIG. 37A. Facepiece 3702 has pad adjustment knob 3700K. Helmet/shell 3710 has a moveable support base 3700SB, a head support/head rest or moveable support 3700SSUS on an adjustable, sliding mount/slide rail for moveable support 3700SR which may be used to adjust head rest angle. Also shown are helmet strap clips 3706A, air adaptor 3700A, and inflation line 3700I.

FIG. 37E is a side view of the vasocompression device of FIG. 37A. Facepiece 3702 has pad adjustment knob 3700K. Helmet/shell 3710 has a moveable support base 3700SB, a head support/head rest or moveable support 3700SSUS on an adjustable, sliding mount/slide rail for moveable support 3700SR which may be used to adjust head rest angle. Also shown are helmet strap clips 3706A, straps 3708, air adaptor 3700A, and inflation line 3700I.

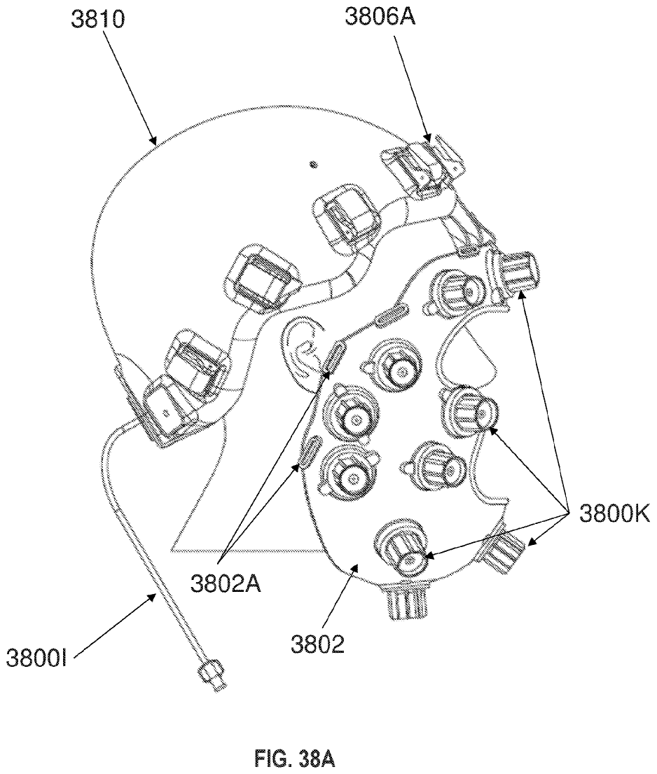

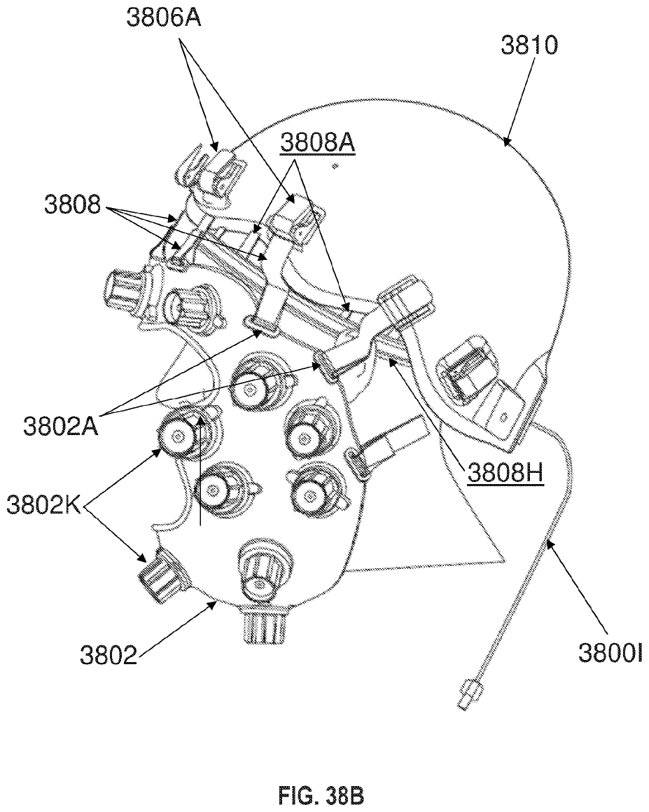

FIG. 38A is a right-side view of a head covering and face support for inducing vasocompression before straps connect the two pieces. As shown here, there are strap support connectors 3806A on the head covering/shell/helmet 3810, an inflation line 1900I, and strap connectors 3802A and knobs 3800K on face support 3802.

FIG. 38B is a left-side view of a head covering and face support for inducing vasocompression with straps connecting the two pieces and an optional headband. As shown here, there are strap support connectors 3806A on the head covering/shell/helmet 3810, an inflation line 3800I, and strap connectors 3802A and knobs 3800K on face support 3802, with straps 3808 connecting strap support connectors 3806A on helmet 3810 to strap connectors 3802A on face support 3802.

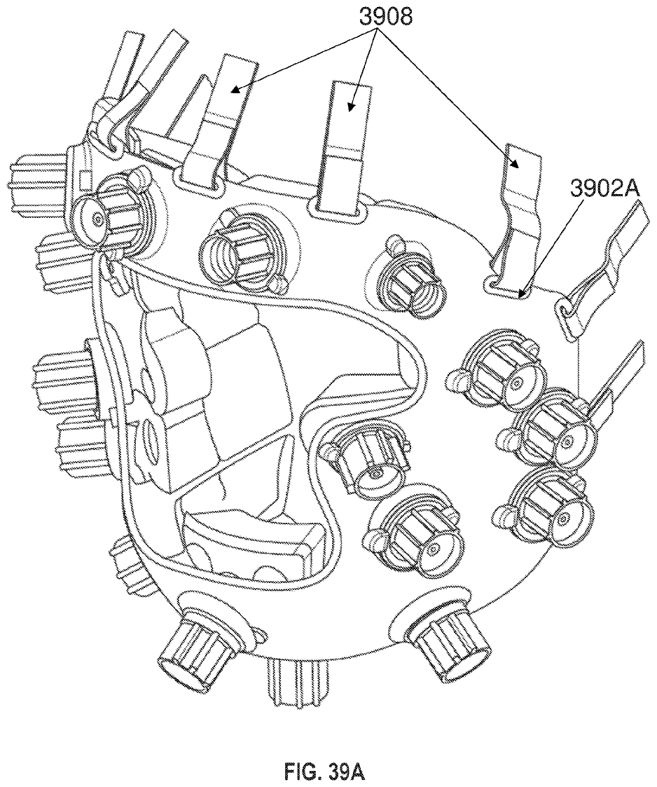

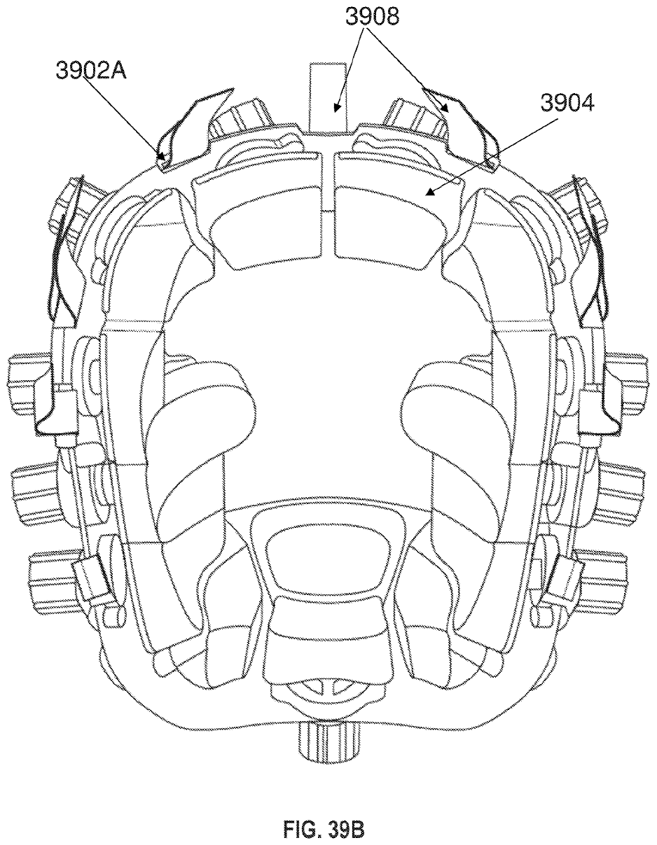

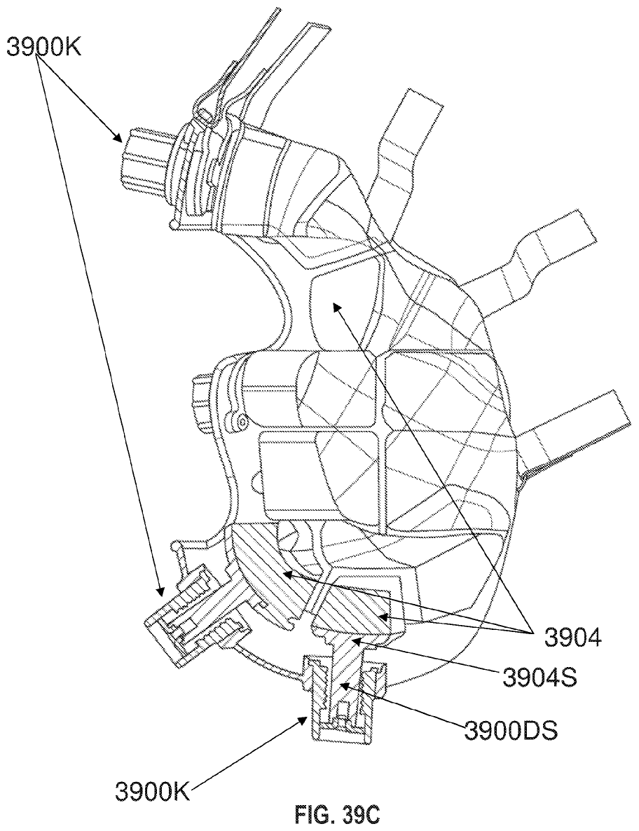

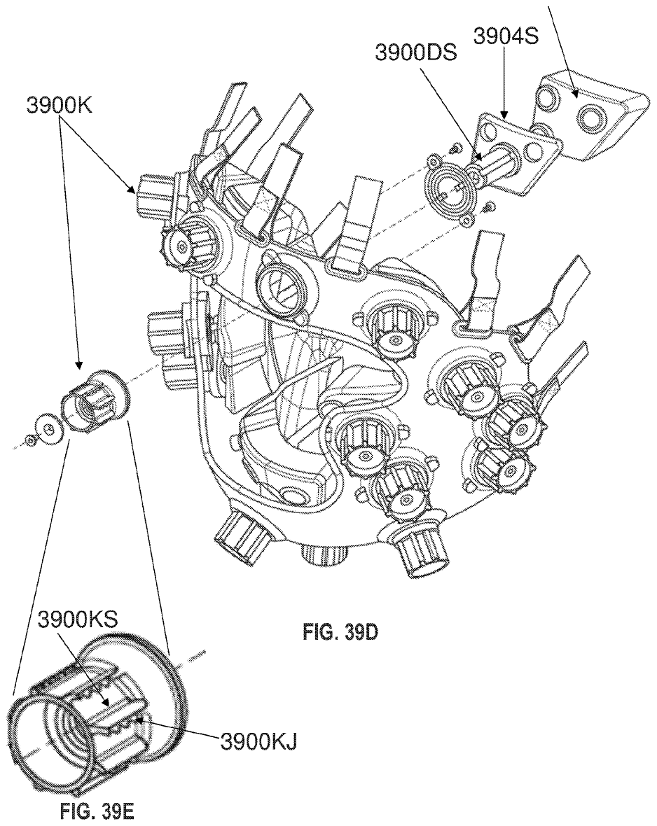

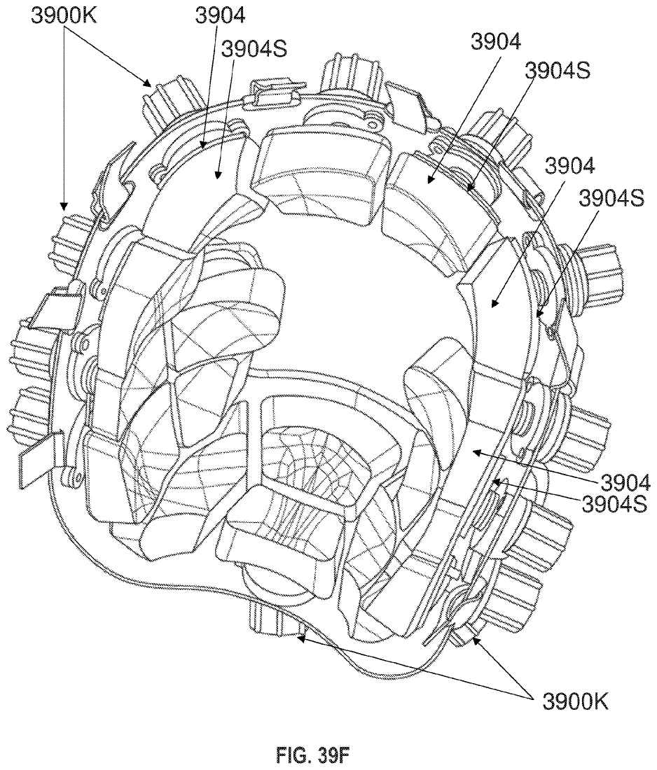

FIG. 39A is an isometric view of a dynamic facepiece having 18 independently controlled support pads. Also shown is strap 3908 and strap connection point on facepiece 3902A.

FIG. 39B is an interior view of the dynamic facepiece of FIG. 39A. Shown here are the independently controlled support pads 3904. As previously mentioned, each pad 3904 or groups of pads 3904 may be sized, contoured, and structured with variable hardness, durometer, and/or stiffness for specific locations on the face and its bony structures.

FIG. 39C is a section view of the dynamic facepiece of FIGS. 39A and 39B with moveable support and various shaped pads. Again shown are pads 3904, pad support 3904S, and pad adjustment knob 3900K coupled to drive shaft 3900DS.

FIG. 39D is an isometric front view showing an exploded view of a pad adjustment knob 3900K in relation to a pad 3904 and pad support 3904S, with a threaded shaft 3900TS, which may be part of drive shaft 3900DS from FIG. 39C, coupled to pad support 36904S.

FIG. 39E illustrates an exploded view of a haptic feedback embodiment of a pad adjustment knob. According to certain embodiments, there may be a haptic feedback feature on one or more or all pad adjustment knobs 3900K, including a smooth edge 3900KS which may correspond to a direction of tightening or increasing pressure of the pads 3904 on a face of the patient via drive shaft 3900DS, and a jagged edge 3900KJ which may correspond to a direction of releasing or decreasing pressure of the pads 3904 on a face of the patient via drive shaft 3900DS. According to yet other embodiments, these directions corresponding to smooth edge 3900KS and jagged edge 3900KJ may be reversed.

FIG. 39F is an isometric interior view of the dynamic facepiece of FIG. 39B. Shown here are the independently controlled support pads 3904, pad supports 3904S and control knobs 3900K. Pairs of specific pad 3904/pad support 3904S combinations vary depending on location.

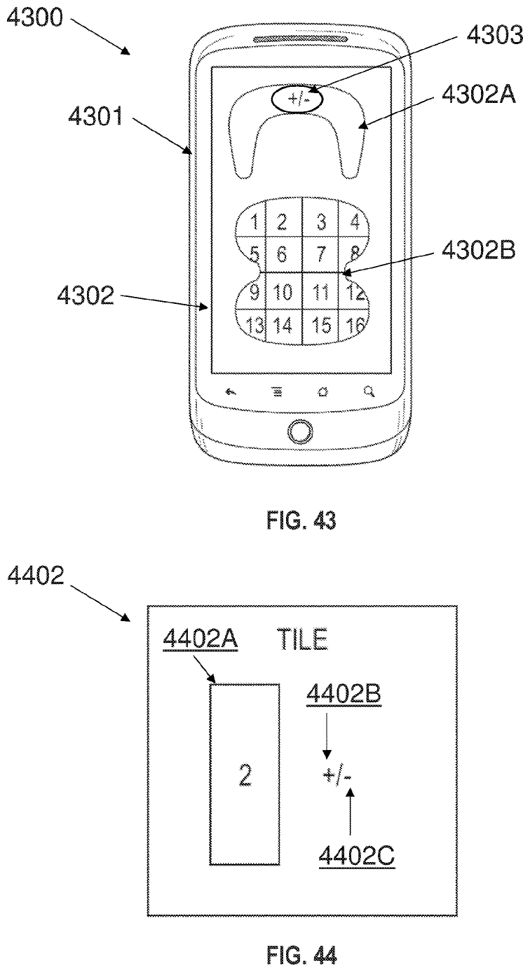

In use, the individual tiles may be adjusted individually by adjusting the pad control knob to increase or decrease pressure of the tile on the face. In additional embodiments, the pad adjustment may be accomplished using any of a variety of mechanized, motorized, pneumatic, electro-mechanical direct, remote, wired or wireless control schemes. Additionally, electronic user interfaces and computer controllers may also be used as described below with regard to FIGS. 43, 44 and 45.

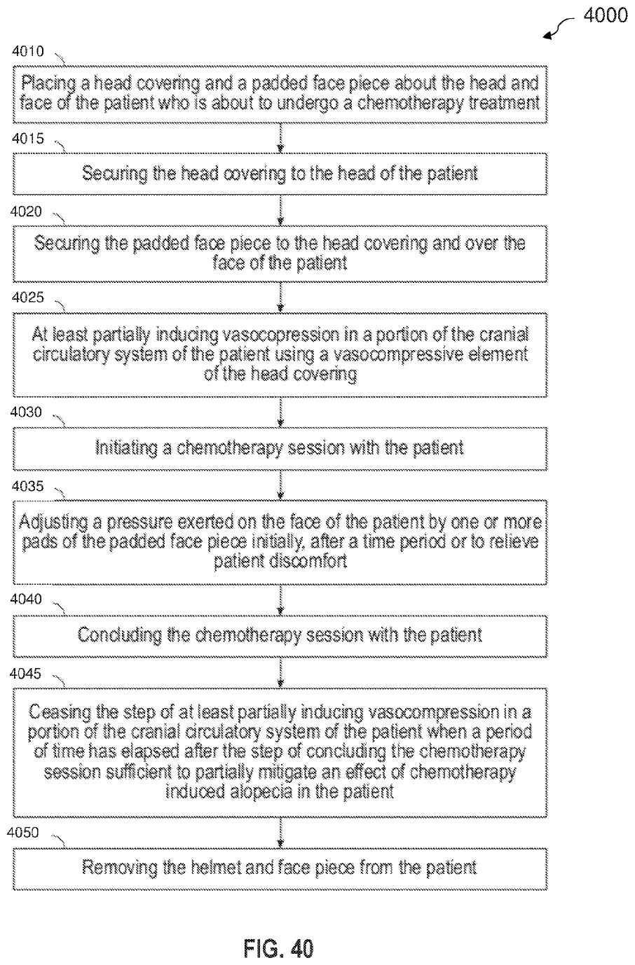

FIG. 40 is a flow chart 4000 of a method of delivering chemotherapy having vasocompression and controllable, adjustable pressure relief of a rigid facepiece.

First, at step 4010 there is placing a head covering on and a facepiece about the head of a patient who is about to undergo a chemotherapy treatment.

Next, at step 4015, there is a step of securing the head covering to the head of the patient.

Next, at step 4020, there is a step of securing the padded face piece to the head covering and over the face of the patient.

Thereafter, there is step 4025, At least partially inducing vasocompression in a portion of the cranial circulatory system of the patient using a vasocompressive element of the head covering.

Then there is the step of Initiating a chemotherapy session with the patient (step 3730).

During the step of providing vasocompression, there is a step of adjusting a pressure exerted on the face of the patient by one or more pads of the padded face piece initially, after a time period or to relieve patient discomfort. This step may be performed according to a program or on patient response for relief of pressure or on other clinical or patient-based indication. (step 4035).

Thereafter, there is the step of concluding the chemotherapy session with the patient (step 4040).

Next, at step 4045, there is the process of ceasing the step of at least partially inducing vasocompression in a portion of the cranial circulatory system of the patient when a period of time has elapsed after the step of concluding the chemotherapy session sufficient to partially mitigate an effect of chemotherapy induced alopecia in the patient.

Finally, at step 4050, there is the process of removing the helmet and face piece from the patient.

FIG. 41 is a perspective view of a representation of the anatomical landmarks and structure of a mandible. The Ramus 4102 joins the mandible body 4104 at the angle of the mandible 4104A also known as the gonial angle. The left and the right mandible body 4104 are joined by the mental protuberance 4106, commonly referred to as the chin. Also shown is the mental tubercle 4106A, mental foramen 4106B, alveolar process 4106C, base of the mandible body 4104B, and oblique line 4104C. Also shown is the head of the mandible 4152, pterygoid fossa 4154, neck of the mandible 4156, mandibular foramen 4158, condylar process 4160, mandibular notch 4162, coronoid process 4164, lingula 4166, mylohyoid groove 4168, sublingual fossa 4170, mylohyoid line 4171, submandibular fossa 4172.

FIGS. 42A-42B illustrate a left side and front view of the bones of the face and skull, respectively. It is to be appreciated that various embodiments of support pads of different shapes, sizes, contours, construction and durometer may be designed to interact with one or more specific anatomical structures because of the ability of that structure to bear the forces produced during vasoconstriction therapy. By way of example and not limitation, as shown in FIG. 42B illustrating a front view of the bones of the face and skull, one or more or an array of support pads may be adapted, contoured, and configured to engage with one or more of a frontal bone 4201, a zygomatic bone 4203, a maxilla 4206 with infraorbital foramen 4206A and anterior nasal spine 4206B, a mandible 4204 with mental foramen 4204A and ramus 4202, and a sphenoid bone 4208 with greater wing 4208A and lesser wing 4208B, lacrimal bone 4210, temporal bone 4212, nasal bone 4214, vomer 4216, inferior nasal concha 4218, and ethmoid bone 4220 including orbital plate 4220A, middle nasal concha 4220B, and perpendicular plate 4220C. Other combinations are possible, and a pad may be configured to engage with and distribute forces across one or more regions of bone shown in FIG. 41 or FIG. 42A-B. For example, FIG. 42A illustrates a left side view of the bones of the face and skull, shown here is the occipital bone 4254, temporal bone 4212 with external acoustic meatus 4212A, parietal bone 4252, coronal suture 4256, sphenoid bone 4208 with greater wing 4208A, frontal bone 4201, ethmoid bone 4220 including orbital plate 4220A, nasal bone 4214, lacrimal bone 4210, zygomatic bone 4203, maxilla 4206 with infraorbital foramen 4206A and anterior nasal spine 4206B, mandible 4204 with mental foramen 4204A, ramus 4202, and coronoid process 4204B. The support pads may also be contoured and customized to variations of the facial bones, facial muscles, and fat pads of patients.

FIG. 43 is an exemplary Graphical User Interface (GUI) for controlling pressure in a head covering and face covering 4300. The user may interact with the GUI 4300 via a display 4301 of a smartphone or controller 4302 in an intuitive manner to select either the inflatable liner 4302A or one or more face piece tiles 4302B. Then, using a +/− or up arrow, down arrow, or increase, decrease icon 4303, the user may adjust the amount of offsetting pressure applied by the vasocompression system. There may also be settings to control the range or amount of increase or decrease for patient safety. Additionally, the system may control amount of adjustment to ensure that vasocompression is maintained to the scalp and eyebrow areas.

FIG. 44 is an exemplary Graphical User Interface (GUI) 4402 for controlling an individual tile on a face covering. In this example, the user has selected tile 2 4402A. On this interface, the + icon 4402B may be used to increase the pressure applied by tile 2 4402A. The − icon 4402C may be used to decrease the pressure applied by tile 2 4402A.

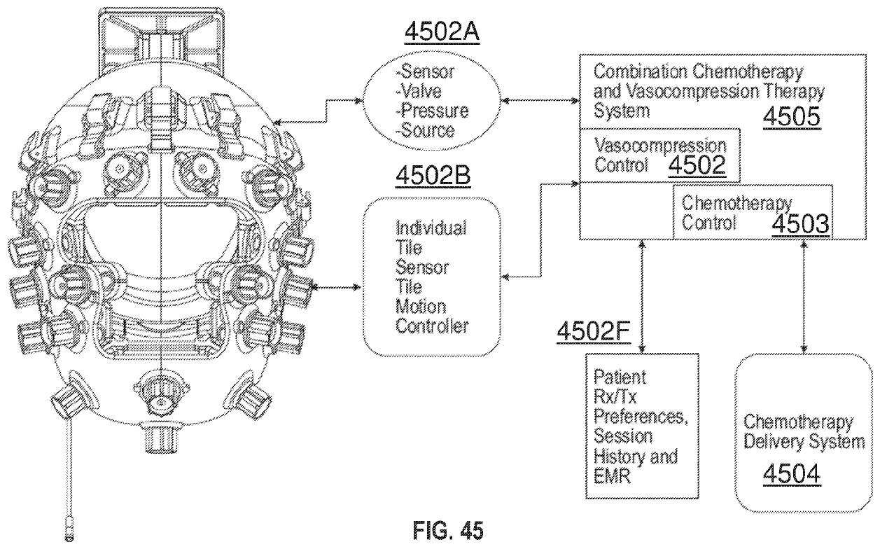

FIG. 45 is a block diagram of an exemplary computer controller for operating a combination chemotherapy and vasocompression therapy system. The vasocompression control system 4502 includes computer components, memory and interfaces suited for interaction and control of the sensors, valves, pressure source 4502A and other elements of the inflatable liner. Additionally, this system will also interact with and control the positioning of each of the tile elements on the face piece, for example via individual tile sensors and tile motion controllers 4502B. It is to be appreciated that the system may be operated in a mode that follows a prior user session and settings/session history 4502F or in a mode that allows user control throughout the session (user preferences) 4502F. Additionally or optionally, the system may operate in a combination mode where an automatic program is running with patient interaction for fine tuning of pressure zones or to indicate where an adjustment is needed which may then be factored into an upcoming zone adjustment. The chemotherapy control system 4503 will interact with the chemotherapy delivery system 4504 as described above in FIGS. 1A-3B to deliver the prescribed agent according to the desired protocol. The system may also be used to automate all or part of the steps of method 1500 or 3700. A patient or healthcare worker may interact with the system of FIG. 45 using the exemplary interfaces as in FIGS. 43 and 44 or other suitable interface including voice, mouse or other computer interface suited to the purpose of providing interaction between the patient and the combination vasocompression and chemotherapy system 4505.

Additionally, the system described in FIG. 45 may provide to and receive from a patient electronic medical record information (EMR) 4502F about the patient prescription 4502F, therapy 4502F, session duration and session history 4502F or other information used by the control system to correctly deliver the chemotherapy agent and preform the chemotherapy procedure.

FIGS. 37A, 38B, 44 and 45 are some examples of embodiments providing a range of variations to adjust the fit and provide comfort. One or more of the following adjustable or user determined aspects may be used to used alone or in a number of combinations to provide comfort or adjust the application of forces used to induce vasocompression: the variable position platform on the top of the helmet, the pressure within the balloon liner/bladder, individual straps around the perimeter of the facepiece as well as user/patient customized and adjustable tiles within the facepiece (see FIGS. 20-25) and overall shape and size of the facepiece. Other adjustable structures may include an additional band 3808A (see FIG. 38B) along with the adjustments via a controller (see FIGS. 43, 44) or manually (see FIG. 45) or combinations thereof.

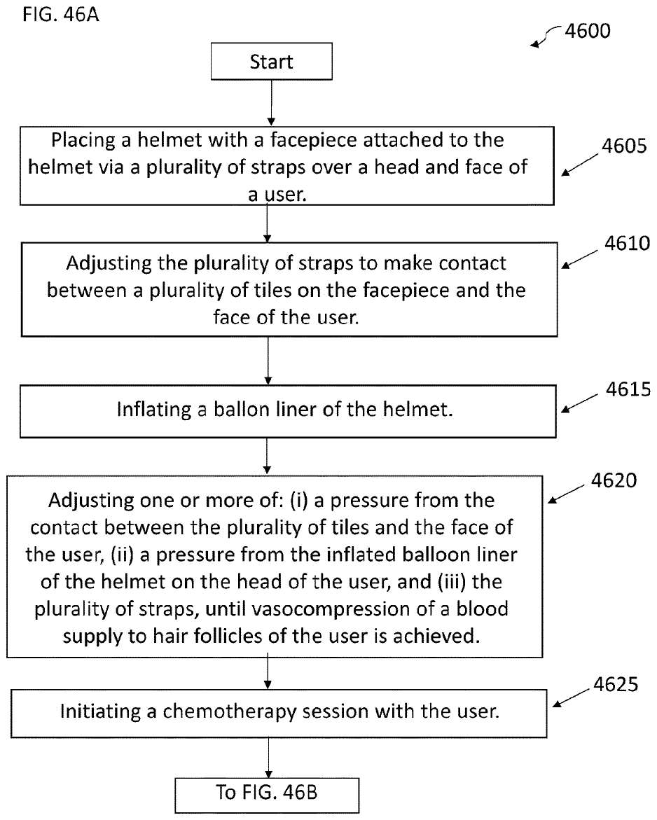

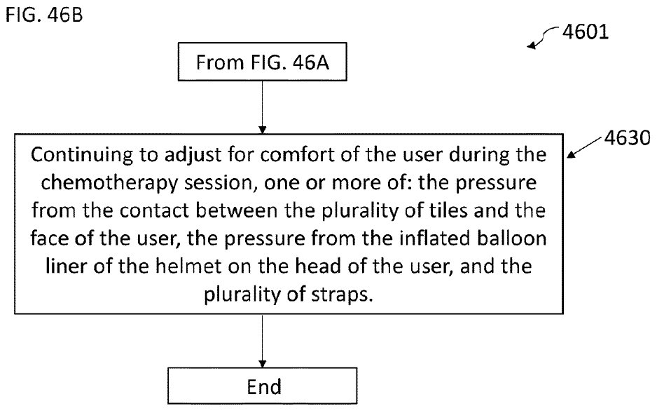

FIGS. 46A-46B is a block diagram illustrating a method for comfort wear of headgear for protecting against hair loss during chemotherapy 4600-4601.

Method 4600-4601 begins at block 4605 of FIG. 46A with placing a helmet with a facepiece attached to the helmet via a plurality of straps over a head and face of a user.

Next, at block 4610, method 4600-4601 continues with adjusting the plurality of straps to make contact between a plurality of tiles on the facepiece and the face of the user.

Next, at block 4615, the balloon liner of the helmet is inflated.

Method 4600-4601 continues at block 4620 with adjusting one or more of: (i) a pressure from the contact between the plurality of tiles and the face of the user, (ii) a pressure from the inflated balloon liner of the helmet on the head of the user, and (iii) the plurality of straps, until vasocompression of a blood supply to hair follicles of the user is achieved.

At block 4625, method 4600-4601 continues with initiating a chemotherapy session with the user.

Method 4600-4601 continues at block 4630 of FIG. 46B with continuing to adjust for comfort of the user during the chemotherapy session, one or more of: the pressure from the contact between the plurality of tiles and the face of the user, the pressure from the inflated balloon liner of the helmet on the head of the user, and the plurality of straps.

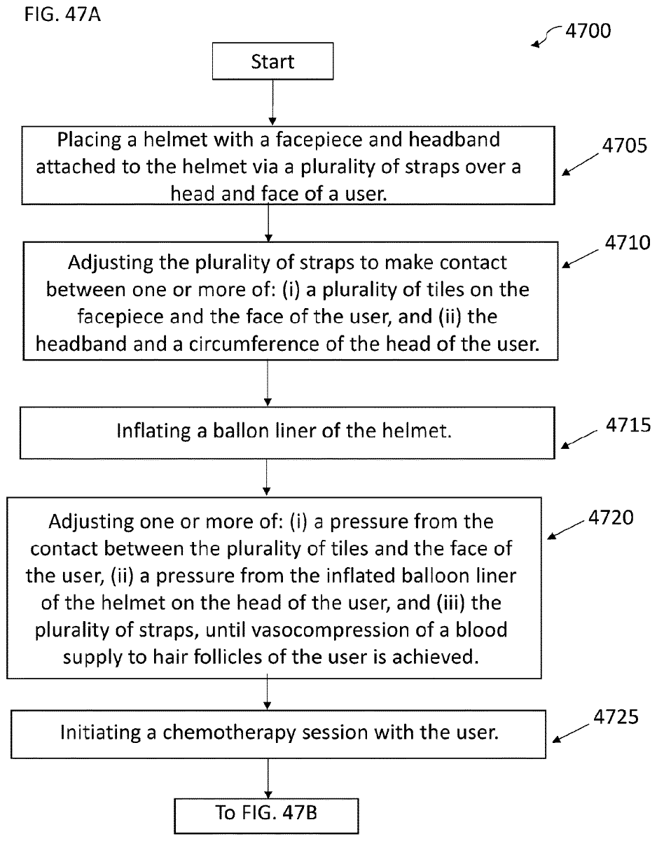

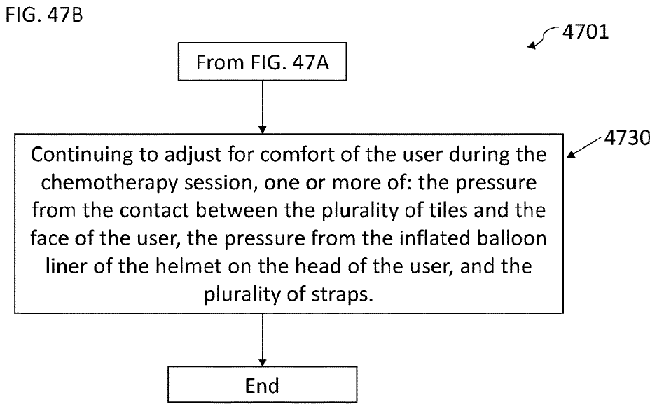

FIGS. 47A-47B are a block diagram illustrating another method for comfort wear of headgear for protecting against hair loss during chemotherapy 4700-4701.

Method 4700-4701 begins at block 4705 of FIG. 47A with placing a helmet with a facepiece and headband attached to the helmet via a plurality of straps over a head and face of a user.

Next, at block 4710, method 4700-4701 continues with adjusting the plurality of straps to make contact between one or more of: (i) a plurality of tiles on the facepiece and the face of the user, and (ii) the headband and a circumference of the head of the user.

Next, at block 4715, the balloon liner of the helmet is inflated.

Method 4700-4701 continues at block 4720 with adjusting one or more of: (i) a pressure from the contact between the plurality of tiles and the face of the user, (ii) a pressure from the inflated balloon liner of the helmet on the head of the user, and (iii) the plurality of straps, until vasocompression of a blood supply to hair follicles of the user is achieved.

At block 4725, method 4700-4701 continues with initiating a chemotherapy session with the user.

Method 4700-4701 continues at block 4730 of FIG. 47B with continuing to adjust for comfort of the user during the chemotherapy session, one or more of: the pressure from the contact between the plurality of tiles and the face of the user, the pressure from the inflated balloon liner of the helmet on the head of the user, and the plurality of straps. In additional alternative embodiments, any of the vasocompression head coverings illustrated and described with regard to FIGS. 9, 10A, 10B, 10C, 11A, 11B, 12A, 12D may be used according to the method of applying a vasocompression counterforce as in FIGS. 21A-21D. Additionally or optionally, these embodiments may also be adapted for use with a rigid mandible support as in FIGS. 16 and 17, a full face support as in FIGS. 18-20 or an embodiment of a counter force support base as in FIGS. 22-25.

For convenience, many different embodiments have been described as using a nylon strap that is connected between two components and then adjusted using a conventional D-ring arrangement to adjust for patient comfort. The various embodiments of the inventive embodiments are not so limited. It is to be appreciated that a strap as used herein may include other configurations of a connector used to maintain relative position between two components of the system. As such, a strap may also include an elastic band, an inelastic band, a spring along or in combination with another element along with different forms of connecting to and bridging the gaps between two components.

Additional details of potentially suitable materials, details of construction of a head covering, a shell, a head covering retention feature or of one or more vasocompression elements may be appreciated by reference to U.S. Pat. No. 3,242,500 to Derr.

Additional details of potentially suitable materials, details of construction of a head covering, a shell, a head covering retention feature or of one or more vasocompression elements may be appreciated by reference to U.S. Pat. No. 9,743,701 to Javorek. By way of illustration and example, there is described herein additional aspects and alternatives for use with one or more head covering retention system embodiments with details related to, for example, a hook and loop fastener, a buckle, a quick release mechanism or an adjustment feature.

Additional details of potentially suitable materials, details of construction of one or more vasocompression elements may be appreciated by reference to US Design Patent 267,287 to Gooding.

Additional details of potentially suitable materials, details of construction of a head covering, a shell, a head covering retention feature or of one or more vasocompression elements may be appreciated by reference to US Patent Publication US 2006/0070170 to Copeland.

Additional details of potentially suitable complimentary compounds which may be used in combination with the devices and methods described herein as well as information related to the treatment of chemotherapy induced alopecia may be appreciated by reference to US Patent Publication US 2019/0240192 to Georgopoulos.

In addition to those listed above, still further additional alternative embodiments and combinations of the innovative features described herein are possible to deliver the active, personalized vasocompressive therapy described herein, such as for example:

Selective vasocompression of the key portions of the blood supply to the scalp—including programmed sequences of applying compression—for example to empty the capillary bed or block the main blood vessels of the scalp/skull.

In one aspect, a head covering is configured to include a set of separate vasocompression elements such as one along the periphery of the head covering or shell and other vasocompression elements within the interior of the shell. In one example, this arrangement would be a peripheral vasocompression zone and a central vasocompression zone. It is to be appreciated that these zones may be operated at different levels of pressure or amount of vasocompression by either manual control or via use of controller 980/1090 (from FIGS. 9-10A, respectively). These exemplary peripheral and central zones may be further divided, or additional other zones may be provided and incorporated into the head covering in order to more precisely deliver vasocompression therapy. Additionally or optionally, these zones may be operated at different pressures or levels of vasocompression as needed for the delivery of a pre-selected or patient specific vasocompression therapy profile.

A single device for protective vasocompression of all hair on the head—advantageously protecting both the hair on the scalp and eyebrows. Inclusion of eyebrows may be provided by additional vasocompressive elements positioned, sized and adapted for this purpose (SEE, e.g., FIGS. 10A and 11A).

A custom fit vasocompression device adapted and configured to patient specific head shape AND hair style. Additional embodiments include use of 3D imaging or scans of the patient head and hair styles, including hair styles to be used during chemotherapy sessions to ensure liner and shell fit for patient comfort and performance of the vasocompression therapy session.

Advanced active control and/or feedback vasocompression control system 980/1090 (from FIGS. 9-10A, respectively) with integrated pressure sensors and other detection devices within/integrated with the vasocompression device, including integration into the liner, expandable layer or shell, including for example stretch, strain or other measurement systems integrated into one or more components of the head covering 1000 (from FIG. 10A) for inducing vasocompression in a patient.

The personalized vasocompression device embodiment may include one or more pressure sensors within an interior portion of a head covering. The one or more pressure sensors can provide an indication of pressure to the control system 180/190. The one or more pressure sensors can be adapted to indicate an amount of pressure applied to a patient undergoing chemotherapy when the patient is wearing the head covering. The head covering can further include one or more sensors to measure skin perfusion (O₂ saturation) or Doppler blood flow monitor or infra-red pulse oximeter sensor.

The inner liner (next to patient) can be a rigid or semi-elastic layer configured for an exact fit for the patient to fit exactly the shape of the patient's head and hair. The head covering retaining system can include a tightening system. The head covering can further include one or more selective pressure points positioned to compress one or more vessels of the cranial circulatory system of a patient. The head covering can further include a vibrating element coupled to the liner or the at least one vasocompression element. The liner and the at least one vasocompression elements can be sized, shaped, or positioned relative to the head covering based on the hair style of the patient.

The head covering can further include a fluid source as used herein to include a liquid or a gas, in communication with the liner or the at least one vasocompression element for inflation of a portion of the liner or a portion of the at least one vasocompression element. The head covering can further include at least one relief valve to prevent over pressurization of an inflatable element of the head covering. The fluid source can be a manual pump mechanism, a bulb, an electric pump, or pressurized tank, cartridge or reservoir. The head covering can further include a control system 980/1090 (from FIGS. 9-10A, respectively) for adjusting the flow of fluid from a fluid source to maintain a desired pressure within the head covering. The desired pressure within the head covering can be sufficient to produce a vasocompression result while minimizing discomfort or side effects to the patient or harm to surrounding structures.

The head covering can further include a cooling fluid source in communication with the liner or the at least one vasocompression element wherein a portion of the liner or a portion of the at least one vasocompression element is adapted to provide cooling to an interior portion of the head covering. Additionally, the fluid used for inflation of a vasocompression element may also be cooled. The head covering can further include one or more pressure sensors within an interior portion of the head covering. The one or more pressure sensors can provide an indication of pressure to the control system 980/1090 (from FIGS. 9-10A, respectively). The one or more pressure sensors can be adapted to indicate an amount of pressure applied to a patient undergoing chemotherapy when the patient is wearing the head covering.

The personalized vasocompression system may also include advanced communication connectivity. As a result of this feature, a user may employ the personal vasocompression device with the functionality found on smart devices while undergoing therapy. Additionally, noise cancellation headphones or ear bud functionally may also be provided to enhance the user experience. The system may also include a display and Bluetooth, near field or other wireless or wired communication capabilities. Still further, the system may communicate status, programming, patient information or data, and/or any vasocompression device data related to all and any other aspects of vasocompression therapy or operations to an onboard storage device, a mobile phone, a communications network, remote network or health information system. In still other aspects, the display may be positioned on the head covering for providing information to a health care provider or the patient. In additional aspects, the head covering may also include ear buds, headphones, speakers or other audio components adjacent to the ears of the patient. In these embodiments, the system is also configured to provide noise cancellation, music, media or other entertainment to the patient while undergoing chemotherapy or using the head covering. Embodiments of the head covering may be externally powered by plugging into a suitable power source or may be powered wirelessly such as by use of an onboard battery system or an onboard rechargeable battery system.

Since it is the intention that hair is protected and preserved by the use of the various vasocompression devices and techniques, it is to be appreciated that aspects of the head covering 1000 (from FIG. 10A) components are modified and adapted for the presence of patient hair 107 (from FIG. 1A). In additional or alternative embodiments, an additional liner or modified vasocompression element may include additional areas of compliance specific to be conformal with and accommodate elevated areas in the skull as well as those areas where the hair is collected or arranged during use of the head covering 1000. In particular, the design and use of the head covering 1000 in the delivery of vasocompression therapy factors in the circumstances that the patient hair, amount, style, placement in relation to the one or more vasocompression elements/manifold 1330 (from FIG. 13D) will compress the hair (which is not compressible) and the hair then will compress the scalp thereby also contributing to the vasocompression of the vasculature.

Additionally or optionally, on or more surfaces of the one or more vasocompression elements may be modified with raised features of a range of different cross section shapes and tips or to have different length protrusions so as to accommodate different regions or points in the specific shape of the patient skull will be appropriately treated even in those regions where it is not on an even surface with the surrounding regions. Additionally, combinations of liner features, vasocompression element features as well as arrangement and placement of the patient's hair may also be used to accommodate patient specific cranial topography and shape to improve operation of the head covering as well as patient specific performance and comfort.

According to various embodiments, an adjustment cycle during chemotherapy treatment is a tradeoff between pressure in balloon liner/bladder against the different parts of the face and head. The user/patient or an assistant or pressure compensation program (FIG. 43, 44, 45) may be used alone or in combination with tightening or loosening of one or more straps between the helmet and the facepiece or the helmet and facepiece and an intermediate pressure strap such as a strap 3808A connected to a headband 3808H (FIG. 38B), along with adjustment of one or more face tiles or the firmness or softness of an adjustable face tile (see FIGS. 20-25).

The various embodiments are intended to provide relief and comfort to the patient undergoing treatment and wearing the helmet. Discomfort created by inducing vasocompression may be provided in a variety of different ways by manipulation of different components of the system, movement or adjustment of the helmet relative to a support structure (see FIGS. 21A-22D) as well as individual or localized adjustment of helmet straps, tiles or modulation of pressure.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected,” “directly attached,” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A head covering for inducing vasocompression in the blood supply of a patient receiving chemotherapy, comprising:
a shell having a front edge, a back edge, a top, a left side edge and a right side edge;
a liner within the shell shaped to at least partially accommodate the hair of the patient;
at least one vasocompression element within the shell or the liner;
a head covering retention system comprising:
a full-face support structure that encircles the mandible body and Ramus, extends across and at least partially covering the Zygomatic, Maxilla and Frontal bones of the skull of the patient with an opening in the full-face support structure for a pair of eyes, a nose and a mouth of the patient;
a cushion along an inner wall of the full-face support between an inner wall of the full-face support and the patient; and
one or more straps between the head covering and the full-face support.

2. The head covering of claim 1, the cushion further comprising:
a plurality of tile face cushions arranged along the inner wall of the full-face support wherein the size, shape, firmness, compression modulus characteristics of each tile face cushion of the plurality of tile face cushions may be selected based on a location within the full-face support and a loading characteristic of the location while vasocompression therapy is being delivered.

3. The head covering of claim 1, further comprising:
a plurality of tile face cushions arranged along the inner wall of the full-face support, wherein each tile face cushion of the plurality of tile face cushions comprises a post; and
a plurality of apertures within the full-face support wherein the location of each aperture is adapted and configured to correspond to a post of a tile face cushion proximate to the aperture.

4. The head covering of claim 1, wherein the one or more straps further comprises
at least two front support straps extending from the front edge of the shell to a top edge of the full-face support; or
at least two right side support straps extending from a right side of the full-face support structure to right side of the shell; or
at least two left side support straps extending from a left side of the full-face support structure to left side of the shell; or any of a combination of the above.

5. The head covering of claim 1, wherein the liner is an inflatable liner.

6. The head covering of claim 5, wherein the inflatable liner has an inner zone against the liner and an outer zone to engage the patient.

7. The head covering of claim 6, wherein a separation zone is provided between the inner zone and the outer zone, the separation zone forming a perimeter that corresponds to the perimeter of the shell.

8. The head covering of claim 6, wherein the separation zone retains its shape during inflation and deflation of the inflatable liner.

9. The head covering of claim 3, wherein each of the plurality of tile face cushions comprises an inner surface contoured to a portion of a face and an outer surface contoured to engage with a portion of the full-face support and a mid-portion between the inner surface and the outer surface.

10. The head covering of claim 9, wherein the mid-portion of each face tile is one or a combination of a gel, a foam, a silicon, a uniform durometer material, a mixed durometer material or an inflation zone.

11. The head covering of claim 9, wherein each face tile has a post for alignment of each face piece tile to a corresponding opening in the full face covering.

12. The head covering of claim 9, wherein each face tile has a shaft and a knob for mechanical adjustment of the spacing of the inner surface of the face tile from the inner surface of the full face covering or a valve for adjusting a pressure within a face tile.

13. A head covering for inducing vasocompression in the blood supply of a patient receiving chemotherapy, comprising:
a shell having a front edge, a back edge, a top, a left side edge and a right side edge;
an inflatable liner within the shell;
a head covering retention system adapted to maintain the position of the shell and the inflatable liner relative to a head of the patient;
a face covering coupled to the shell; and
at least one pad support within the face covering.

14. The device of claim 13, wherein the face covering includes a plurality of connection sites providing access for a plurality of support pads within the face covering.

15. The device of claim 14, wherein the at least one pad support is a plurality of individual pads adapted and configured to at least partially conform to a bony structure of the patient.

16. The device of claim 15, wherein the plurality of individual pads is controllable in applying pressure to a portion of the patient's face using a mechanical adjustment system.

17. The device of claim 15, wherein the plurality of individual pads is controllable in applying pressure to a portion of the patient's face using an electrotechnical adjustment system.

18. The device of claim 17, wherein the electrotechnical adjustment system further comprising a time based or used selected program of increasing or decreasing the pressure applied by a pad to a portion of the patient's face.

19. The device of claim 18, wherein the user selected program is controlled using a control interface, remote device, smart device or app selected by the user based on pre-set pressure settings, prior chemotherapy sessions or based on time-based adjustments to relieve intermittent fatigue while using the vasocompression device.

20. The head covering of any of claims 13-19, further comprising a vasocompression counterforce structure is a ring having an inner diameter from 1 inch to 4 inches.

21. The head covering of claim 20, wherein the vasocompression counterforce structure has a frustoconical shape with a base having an outer diameter of 6 to 8 inches and an upper surface having a ring with a diameter ranging from 1 inch to 4 inches.

22. The head covering of claim 20, wherein the vasocompression counterforce structure has a base, a stem and an upper support surface, wherein a diameter of the base is from 1-3 inches.

23. The head covering of claim 20, wherein the vasocompression counterforce structure has a base, an elongated stem and a rectangular upper support surface.

24. The head covering of claim 20, wherein the vasocompression counterforce structure has a frustoconical shape with a base having an outer diameter of 6 to 8 inches and an upper ring supporting a base, an elongated stem and an upper support surface.

25. A head covering for inducing vasocompression in the blood supply of a patient receiving chemotherapy, comprising:
a shell having a front edge, a back edge, a top, a left side edge and a right side edge;
at least one vasocompression element within the shell;
a head covering retention system adapted to maintain the position of the shell and liner relative to a head of the patient; and
a vasocompression counterforce structure coupled to the shell.

26. The head covering of claim 25, wherein the vasocompression counterforce structure is a ring having an inner diameter from 1 inch to 4 inches.

27. The head covering of claim 25, wherein the vasocompression counterforce structure has a frustoconical shape with a base having an outer diameter of 6 to 8 inches and an upper surface having a ring with a diameter ranging from 1 inch to 4 inches.

28. The head covering of claim 25, wherein the vasocompression counterforce structure has a base, a stem and an upper support surface, wherein a diameter of the base is from 1-3 inches.

29. The head covering of claim 25, wherein the vasocompression counterforce structure has a base, an elongated stem and a rectangular upper support surface.

30. The head covering of claim 25, wherein the vasocompression counterforce structure has a frustoconical shape with a base having an outer diameter of 6 to 8 inches and an upper ring supporting a base, an elongated stem and an upper support surface.

31. A head covering for inducing vasocompression in the blood supply of a patient receiving chemotherapy, comprising:
a shell having a front edge, a back edge, a top, a left side edge and a right side edge;
a liner within the shell shaped to at least partially accommodate the hair of the patient;
at least one vasocompression element within the shell;
a head covering retention system comprising:
a submandibular rigid support extending from a left side of a mandible body around the mental protuberance to a right side of a mandible body;
a cushion on submandibular rigid support;
a front left support strap extending from the front edge of the shell to the submandibular rigid support;
a front right support strap extending from the front edge of the shell to the submandibular rigid support;
a left-side support strap extending from the left-side edge of the shell to the left end of the submandibular rigid support;
a right-side support strap extending from the right-side edge of the shell to the right end of the submandibular rigid support.

32. The head covering of claim 31, wherein the cushion extends above the submandibular rigid support and beyond the left and right ends of the submandibular rigid support.

33. The head covering of claim 31, wherein the cushion is a foam filler within the structure of the submandibular rigid support.

34. The head covering of claim 31, wherein the submandibular rigid support is extending from a position proximate to a left side angle of the mandible, along the left side of a mandible body around the mental protuberance, along the right side of a mandible body to a position proximate to a right-side angle of the mandible.

35. The head covering of any one of claims 31-34, further comprising a vasocompression counterforce structure of any one of claims 25-30, with a dynamic facepiece of any one of claims 13-24.

36. The head covering of any one of claims 1-12, further comprising a vasocompression counterforce structure of in any one of claims 25-30, or of a dynamic facepiece of any one of claims 13-24.

37. The head covering of any one of claims 1-34, wherein the head covering retention system comprises one or more of a rear strap, a front strap or a neck strap connected to the shell.

38. The head covering of claim 37, wherein the shell retention system comprises one or more adjustment elements.

39. The head covering of claim 37, wherein the at least one vasocompression element is an expandable bladder within the liner.

40. The head covering of claim 37, wherein the at least one vasocompression element is coupled to the shell.

41. The head covering of claim 39, further comprising a pressure source in communication with the expandable bladder.

42. The head covering of claim 37, further comprising a continuous perimeter connecting the front edge, the back edge, the left side edge and the right side edge.

43. The head covering of claim 37, the at least one vasocompression element further comprising an expandable feature responsive to a pressure source to expand away from the shell.

44. The head covering of claim 37, further comprising an expandable layer coupled to the liner wherein the at least one vasocompression element is on the expandable layer.

45. The head covering of claim 37, the at least one vasocompression element further comprising one or more raised features.

46. The head covering of claim 37, the liner within the shell shaped to at least partially accommodate the hair of the patient further comprising one or more features on the liner to engage with the hair of the patient.

47. The head covering of claim 37, wherein the liner or the at least one vasocompression element are smooth.

48. The head covering of claim 37, wherein the liner or the at least one vasocompression element comprise an array of evenly distributed protrusions having straight or circular inflatable structures.

49. The head covering of claim 37, wherein the head covering retention system further comprising: a hook and loop fastener, a buckle, a quick release mechanism or an adjustment feature.

50. The head covering of any one of claims 1-34, further comprising a gas source in communication with the liner or the at least one vasocompression element for inflation of a portion of the liner or a portion of the at least one vasocompression element.

51. The head covering of claim 50, further comprising at least one relief valve to prevent over pressurization of an inflatable element of the head covering.

52. The head covering of claim 50, wherein the gas source is a manual pump mechanism, a bulb, an electric pump, or pressurized tank, cartridge or reservoir.

53. The head covering of claim 50, further comprising a control system for adjusting the flow of gas from the gas source to maintain a desired pressure within the head covering.

54. The head covering of claim 53, wherein the desired pressure within the head covering is sufficient to produce a vasocompression result while minimizing discomfort or side effects to the patient or harm to surrounding structures.

55. The head covering of claim 50, further comprising a cooling gas source in communication with the liner or the at least one vasocompression element wherein a portion of the liner or a portion of the at least one vasocompression element is adapted to provide cooling to an interior portion of the head covering.

56. The head covering of claim 50, further comprising one or more pressure sensors within an interior portion of the head covering.

57. The head covering of claim 56, wherein the one or more pressure sensors provide an indication of pressure to the control system of claim 19.

58. The head covering of claim 56, wherein the one or more pressure sensors are adapted to indicate an amount of pressure applied to a patient undergoing chemotherapy when the patient is wearing the head covering.

59. The head covering of claim 50, further comprising one or more sensors to measure skin perfusion (O₂ sat) or Doppler blood flow monitor or infra-red pulse oximeter sensor or for confirmation that blood flow in vessels undergoing vasocompression has stopped or slowed sufficiently.

60. The head covering of any one of claims 1-34, further comprising a continuous structure from the occiput area to the eyebrows or to the forehead.

61. The head covering of claim 60, further comprising a structure or an inflatable structure to compress the eyebrows for a vasocompression effect on the eyebrows.

62. The head covering of claim 60, further comprising a timer and a control system adapted to provide an automatic deflation protocol when the timer has elapsed or to cease operation of any active vasocompression element.

63. The head covering of claim 60, further comprising a timer and a control system adapted for programmed automatic inflation and deflation of the liner or the one or more vasocompression elements or the activation and deactivation of the one or more vasocompression elements.

64. The head covering of claim 60, wherein the liner is a rigid or semi-elastic layer configured for an exact fit for the patient to fit exactly the shape of the patient's head and hair.

65. The head covering of claim 64, wherein the head covering retaining system comprises a tightening system.

66. The head covering of claim 60, further comprising one or more selective pressure points positioned to compress one or more vessels of the cranial circulatory system of a patient.

67. The head covering of claim 60, further comprising a vibrating element coupled to the liner or the at least one vasocompression element.

68. The head covering of claim 60, wherein the liner and the at least one vasocompression elements are sized, shaped, or positioned relative to the head covering based on the hair style of the patient.

69. A head covering for inducing vasocompression in the blood supply of a patient receiving chemotherapy, comprising:
a cap having a front edge, a back edge, a top, a left side edge and a right side edge;
a liner within the cap shaped to at least partially accommodate the hair of the patient; and
at least one vasocompression element within the cap.

70. The head covering of claim 69, wherein the liner further comprises a first layer adjacent to the inner surface of the cap and a second layer adapted and configured to engage with the patient's head and a separation zone separating the first layer from the second layer wherein the separation zone has a perimeter that corresponds to the edges of the cap.

71. The head covering of claim 69, wherein the cap comprises a flexible structure, a partially flexible structure, a combination of semi-rigid structure and flexible structure or an adjustable structure sized to remain in place on the head when the at least one vasocompression element is active.

72. The head covering of claim 69, wherein the at least one vasocompression element is an expandable bladder within the cap or the liner.

73. The head covering of claim 69, wherein the at least one vasocompression element is coupled to the cap or the liner.

74. The head covering of any one of claims 69-73, further comprising a pressure source in communication with the expandable bladder or the at least one vasocompression element.

75. The head covering of claim 69, further comprising a continuous perimeter connecting the front edge, the back edge, the left side edge and the right side edge.

76. The head covering of claim 69, the at least one vasocompression element further comprising an expandable feature responsive to a pressure source to expand away from the cap.

77. The head covering of claim 69, further comprising an expandable layer coupled to the liner wherein the at least one vasocompression element is on the expandable layer.

78. The head covering of claim 69, the at least one vasocompression element further comprising one or more raised features.

79. The head covering of claim 69, the liner within the shell shaped to at least partially accommodate the hair of the patient further comprising one or more features on the liner to engage with the hair of the patient.

80. The head covering of claim 69, wherein the liner or the at least one vasocompression element are smooth.

81. The head covering of claim 69, wherein the liner or the at least one vasocompression element comprise an array of evenly distributed protrusions having straight or circular inflatable structures.

82. The head covering of claim 69, further comprising a gas source in communication with the liner or the at least one vasocompression element for inflation of a portion of the liner or a portion of the at least one vasocompression element.

83. The head covering of claim 82, further comprising at least one relief valve to prevent over pressurization of an inflatable element of the head covering.

84. The head covering of claim 82, wherein the gas source is a manual pump mechanism, a bulb, an electric pump, or pressurized tank, cartridge or reservoir.

85. The head covering of claim 82, further comprising a control system for adjusting the flow of gas from the gas source to maintain a desired pressure within the head covering.

86. The head covering of claim 85, wherein the desired pressure within the head covering is sufficient to produce a vasocompression result while minimizing discomfort or side effects to the patient or harm to surrounding structures.

87. The head covering of claim 69, further comprising a cooling gas source in communication with the liner or the at least one vasocompression element wherein a portion of the liner or a portion of the at least one vasocompression element is adapted to provide cooling to an interior portion of the head covering.

88. The head covering of claim 69, further comprising one or more pressure sensors within an interior portion of the head covering.

89. The head covering of claim 88, wherein the one or more pressure sensors provide an indication of pressure to the control system of claim 50.

90. The head covering of claim 88, wherein the one or more pressure sensors are adapted to indicate an amount of pressure applied to a patient undergoing chemotherapy when the patient is wearing the head covering.

91. The head covering of claim 69, further comprising one or more sensors to measure skin perfusion (O₂ sat) or Doppler blood flow monitor or infra-red pulse oximeter sensor.

92. The head covering of claim 69, further comprises a continuous structure from the occiput area to the eyebrows or to the forehead.

93. The head covering of any one of claims 69-72, further comprising a structure or an inflatable structure to compress the eyebrows for a vasocompression effect on the eyebrows.

94. The head covering of claim 69, further comprising a timer and a control system adapted to provide an automatic deflation protocol when the timer has elapsed or to cease operation of any active vasocompression element.

95. The head covering of claim 69, further comprising a timer and a control system adapted for programmed automatic inflation and deflation of the liner or the activation and deactivation of the one or more vasocompression elements.

96. The head covering of claim 69, wherein the liner is a rigid or semi-elastic layer configured for an exact fit for the patient to fit exactly the shape of the patient's head and hair.

97. The head covering of claim 96, wherein the head covering retaining system comprises a tightening system.

98. The head covering of any one of claims 69-97, further comprising one or more selective pressure points positioned to compress one or more vessels of the cranial circulatory system of a patient.

99. The head covering of claim 98, further comprising a vibrating element coupled to the liner or the at least one vasocompression element.

100. A method of inducing vasocompression in a cranial circulatory system of a patient receiving chemotherapy, comprising:
placing a head covering about the head of the patient;
at least partially inducing vasocompression in a portion of the cranial circulatory system of the patient using a vasocompression element of the head covering;
inducing a counter force applied to the head covering sized and directed to offset at least some of the forces introduced to the head of the patient as a result of the at least partially inducing vasocompression step;
initiating a chemotherapy session with the patient;
concluding the chemotherapy session with the patient; and
ceasing the step of at least partially inducing vasocompression in a portion of the cranial circulatory system of the patient when a period of time has elapsed after the step of concluding the chemotherapy session sufficient to partially mitigate an effect of chemotherapy induced alopecia in the patient.

101. The method of claim 100, wherein the step of inducing a counter force applied to the head covering is performed by the force acting directly on the shell of the head covering.

102. The method of claim 100, wherein the step of inducing a counter force applied to the head covering is performed by the force acting on a vasocompression counterforce structure coupled to the shell of the head covering.

103. The method of claim 100, further comprising engaging a head covering retention system to immobilize the head covering relative to the head of the patient.

104. The method of claim 100, wherein the period of time that has elapsed after the step of concluding the chemotherapy session up to 24 hours.

105. The method of claim 100, wherein the period of time that has elapsed after the step of concluding the chemotherapy session is at least one hour.

106. The method of claim 100, wherein the period of time that has elapsed after the step of concluding the chemotherapy session is at least 48 hours.

107. The method of claim 100 wherein the vasocompression element of the head covering is a liner wherein a step of expanding the liner or a step of contracting the liner is performed during the step of at least partially inducing vasocompression in a portion of the cranial circulatory system of the patient.

108. The method of claim 107, wherein there is an increase in the amount of vasocompression during the step of expanding the liner corresponding to an increase of the force of the liner against the scalp of the patient.

109. The method of claim 107, wherein there is a decrease in the amount of vasoconstriction during the step of contracting the liner corresponding to a decrease of the force of the liner against the scalp of the patient.

110. The method of claim 100, wherein the vasocompression element of the head covering is a liner having one or more features wherein a step of expanding the liner or a step of contracting the liner is performed during the step of at least partially inducing vasocompression in a portion of the cranial circulatory system of the patient.

111. The method of claim 110, wherein there is an increase in the amount of vasocompression during the step of expanding the liner corresponding to an increase of the force of the one or more features on the liner against the scalp of the patient.

112. The method of claim 110, wherein there is a decrease in the amount of vasocompression during the step of contracting the liner corresponding to a decrease of the force of the one or more features on the liner against the scalp of the patient.

113. The method of claim 100, wherein the vasocompression element of the head covering is an inflatable bladder wherein the degree of vasocompression during the step of at least partially inducing vasocompression in a portion of the cranial circulatory system of the patient corresponds at least in part to the amount of pressure within the inflatable bladder.

114. The method of claim 100, wherein the vasocompression element of the head covering is an inflatable bladder wherein the degree of vasocompression during the step of at least partially inducing vasocompression in a portion of the cranial circulatory system of the patient is increased or decreased by raising or lowering the pressure in the inflatable bladder against the skull.

115. The method of claim 100, wherein the vasocompression element of the head covering is one or more features arranged on an interior surface of the head covering which are urged into contact with a portion of the patient's scalp having hair follicles during the step of at least partially inducing vasocompression in a portion of the cranial circulatory system of the patient.

116. The method of claim 100, wherein the vasocompression element of the head covering is one or more features arranged on an interior surface of the head covering which correspond to an arrangement of the patient's hair, wherein the one of more features act on the arrangement of the patient's hair to further the performance of the step of least partially inducing vasocompression in a portion of the cranial circulatory system of the patient.

117. The method of claim 100, wherein during the at least partially inducing vasocompression step the vasocompression element of the head covering compresses a portion of the scalp containing a plurality of hair follicles against a portion of the cranium to at least partially vasocompress the blood supply to the plurality of hair follicles.

118. The method of claim 117, wherein the portion of the cranium is one or more of a superior temporal line, an inferior temporal line, a parietal bone, a squamous suture, a temporal bone, a lambdoid suture, an occipital bone, a mastoid process, a sphenoid, a portion in proximity to the supraorbital foramen, a frontal bone, a coronal suture.

119. The method of claim 100, wherein during the at least partially inducing vasocompression step the vasocompression element of the head covering is positioned to at least partially vasocompress an artery or a vein of the cranial circulatory system of the patient by compressing the artery or the vein against a portion of the cranium.

120. The method of claim 119, wherein the artery or the vein of the cranial circulatory system is a branch of an external carotid artery or a branch of the internal carotid artery.

121. The method of claim 119, wherein the artery or the vein of the cranial circulatory system is an ophthalmic artery.

122. The method of claim 119, wherein the artery or the vein of the cranial circulatory system is a superficial temporal artery.

123. The method of claim 119, wherein the artery or the vein of the cranial circulatory system is a posterior auricular artery.

124. The method of claim 119, wherein the artery or the vein of the cranial circulatory system is an occipital artery.

125. The method of claim 119, wherein the artery or the vein of the cranial circulatory system is a supraorbital artery or a supratrochlear artery.

126. The method of claim 119, wherein the portion of the cranium is one or more of a superior temporal line, an inferior temporal line, a parietal bone, a squamous suture, a temporal bone, a lambdoid suture, an occipital bone, a mastoid process, a sphenoid, a portion in proximity to the supraorbital foramen, a frontal bone, a coronal suture.

127. The method of claim 100 wherein the step of at least partially inducing vasocompression in a portion of the cranial circulatory system results from compression of a portion of the scalp containing a blood vessel of the cranial circulatory system of the patient against a portion of the periosteum of the patient.

128. The method of claim 100, wherein the step of at least partially inducing vasocompression in a portion of the cranial circulatory system is produced by interaction between the vasocompression element of the head covering and a blood vessel of the cranial circulatory system of the patient.

129. The method of claim 100, the step of at least partially inducing vasocompression in a portion of the cranial circulatory system further comprising compressing a portion of the scalp against a portion of the periosteum to induce vasocompression in a portion of a vascular bed of a hair follicle.

130. The method of claim 100, wherein the vasocompression element of the head covering is positioned with respect to the patient's head wherein during the engaging step the vasocompression element is positioned in proximity a specific vein or a specific artery of the cranial circulatory system of the patient to at least partially vasocompress the specific vein or the specific artery.

131. The method of claim 130, wherein the specific vein or the specific artery is any of the blood vessels in any one of the claims 120-125.

132. The method of claim 100, further comprising delivery of a vasoconstrictor medication to the patient before, during or after any step of claim 66.

133. The method of claim 130, wherein the vasoconstrictor medication is one of an alpha-adrenoceptor agonist, a vasopressin analog, an epinephrine, a norepinephrine, a phenylephrine, a dopamine, a dobutamine, a migraine medication, a headache medication, a serotonin 5-hydroxytryptamine agonist, and a triptans.

134. The method of any one of claims 100-133, wherein the patient is receiving chemotherapy by an injection, an infusion, a targeted therapy, a hormone therapy or an immunotherapy.

135. The method of claim 134, wherein the patient is receiving chemotherapy by an intravenous method, an intrathecal method, an intra-arterial method, an intracavitary method, an intramuscular method, an intralesional method, or an intravesical method.

136. The method of claim 134 or claim 135, wherein the patient is receiving chemotherapy in a home, a health care provider office, a clinic, an outpatient infusion center, a hospital infusion center, or a hospital in-patient room.

137. A method for comfort wear of headgear to protect against hair loss during chemotherapy, the method comprising:
placing a helmet with a facepiece attached to the helmet via a plurality of straps over a head and face of a user;
adjusting the plurality of straps to make contact between a plurality of tiles on the facepiece and the face of the user;
inflating a balloon liner of the helmet;
adjusting one or more of: (i) a pressure from the contact between the plurality of tiles and the face of the user, (ii) a pressure from the inflated balloon liner of the helmet on the head of the user, and (iii) the plurality of straps, until vasocompression of a blood supply to hair follicles of the user is achieved;
initiating a chemotherapy session with the user; and
continuing to adjust for comfort of the user during the chemotherapy session, one or more of: the pressure from the contact between the plurality of tiles on the face and the user, the pressure from the inflated balloon liner of the helmet on the head of the user, and the plurality of straps.

138. The method of claim 137, wherein the plurality of tiles have an outer surface facing an interior of the facepiece and an inner surface facing the face of the user.

139. The method of claim 137, wherein the balloon liner is inflated via an air adaptor coupled to an inflation line and inflation source.

140. The method of claim 137, wherein adjusting the pressure from the contact between the plurality of tiles on the facepiece comprises engaging a knob having a plurality of ridges each having a smooth edge and a jagged edge, wherein the smooth edge is configured to indicate a first direction of rotation to increase the pressure of the contact between the plurality of tiles and the face of the user by moving one or more of the plurality of tiles towards the face of the user, wherein the jagged edge is configured to indicate a second direction of rotation to decrease the pressure of the contact between the plurality of tiles and the face of the user by moving one or more of the plurality of tiles away from the face of the user.

141. The method of claim 137, wherein the knob interfaces with padding and a support piece to increase or decrease one or more of: (i) a distance between the padding/support piece and the face of a patient, and (ii) a pressure on the face of the patient, wherein the padding is a low durometer silicon pad having 5-20 units Shore A.

142. The method of claim 137, wherein the inflatable ballon liner has a first zone configured to interface with the head of the patient, a second zone configured to interface with an interior surface of the helmet and a boundary layer between the first zone and the second zone, wherein the second zone and the boundary layer each have a higher durometer than a durometer of the first zone of the balloon liner.

143. The method of claim 137, wherein the first zone has a thickness of 10-100 microns, wherein the second zone has a thickness of 50-300 microns.

144. The method of claim 143, wherein the boundary layer is of a consistent width in a range of 20-40 mm around a perimeter of the helmet between the first zone and the second zone.

145. The method of claim 137, wherein one or more of the facepiece and a position of the plurality of tiles is pre-contoured to a facial topography of the user prior to putting the facepiece on the face of the user.

146. The method of claim 137, further comprising a waiting period to confirm vasocompression of the blood supply to the hair follicles of the user before initiating the chemotherapy session with the user.

147. The method of claim 137, further comprising offsetting load and pressure on the head or face of the user by resting one or more of the helmet and facepiece being worn by the user against a support surface to generate a desired counter force vector.

148. The method of claim 137, further comprising adjusting one or more of the pressure from the contact between the plurality of tiles on the face and the user, the pressure from the inflated balloon liner of the helmet (inflation state) on the head of the user, and the plurality of straps before or during the chemotherapy session manually or automatically via a graphical user interface (GUI) of a computer implemented application or a web application, wherein the GUI adjusts one or more of the plurality of tiles, one or more of the plurality of straps, and an inflation state of the balloon liner.

149. The method of claim 148, wherein the GUI adjusts the one or more of the plurality of tiles, one or more of the plurality of straps, and the inflation state of the balloon liner via interfacing with a combination chemotherapy and vasocompression therapy control and delivery system via sensors and valves on the helmet, balloon liner, facepiece, plurality of tiles, and pressure sources, further wherein the combination chemotherapy and vasocompression therapy control and delivery system is responsive to user prescription and treatment preferences, chemotherapy session history, and electronic medical records (EMR) of the user.

150. A method for comfort wear of headgear to protect a user against hair loss during chemotherapy, the method comprising:
placing a headband about the head of the user;
placing a helmet with a facepiece and headband attached to the helmet via a plurality of straps over a head and face of the user so that the headband is between the helmet and the facepiece;
adjusting the plurality of straps to make contact between one or more of: (i) a plurality of tiles on the facepiece and the face of the user, and (ii) the headband and a circumference of the head of the user;
inflating a balloon liner of the helmet;
adjusting one or more of: (i) a pressure from the contact between the plurality of tiles and the face of the user, (ii) a pressure from the inflated balloon liner of the helmet on the head of the user, and (iii) the plurality of straps, until vasocompression of a blood supply to hair follicles of the user is achieved;
initiating a chemotherapy session with the user; and
continuing to adjust for comfort of the user during the chemotherapy session, one or more of: the pressure from the contact between the plurality of tiles and the face of the user, the pressure from the inflated balloon liner of the helmet on the head of the user, and the plurality of straps.

151. The method of claim 150, wherein the plurality of straps include one or more of straps, bands, and adhesive, wherein the headband is configured to perform one or more of: (i) stabilizing the helmet during inflation and deflation of the balloon liner, (ii) offsetting a load on the helmet, and (iii) further restricting circulation of blood to hair follicles of the patient.

152. The method of claim 150, wherein the headband is separately put on the forehead of the user and positioned to be located at a gap between the helmet and the facepiece.

153. A device for protecting against hair loss during chemotherapy, the device comprising: a helmet having an inflatable balloon liner and a plurality of helmet connection points, a facepiece having a plurality of facepiece connection points at a perimeter of the facepiece, a plurality of straps to connect the facepiece to the helmet via the plurality of helmet connection points and facepiece connection points.

154. The device of claim 153, further comprising a headband coupled to the helmet.

155. The device of claim 154, further comprising the inflatable ballon liner having:
a first zone configured to interface with the head of the patient, and
a second zone configured to interface with an inner surface of the helmet, wherein the first zone has a lower durometer than a durometer of the second zone of the balloon liner and a boundary zone between the first zone and the second zone wherein the boundary zone follows a perimeter of the helmet.

156. The device of claim 155, wherein the facepiece has a plurality of tiles, wherein the tiles are comprised of materials including one or more of foam, foam composite, gel, air, wherein one or more of the tiles are configured to be adjusted via mechanical or pneumatic mechanisms.

157. The method of any one of claims 100-136 or claims 137-149 or claims 150-152 performed using a device as in any one of claim 36 or claim 36 or claim 37 or claim 50 or any one of claims 69-99 or any one of claims 153-156.

158. A computer controller adapted and configured to control the pressure applied to the scalp and face a patient using any of the above described methods using any one or a combination of the above devices before, during and after a chemotherapy session.

159. A graphical user interface adapted and configured for interaction with the computer controller of claim 158 that enables selection of one or more of an inflatable element or pressure applying element that may be increased or decreased.

160. The computer controller of claim 158 or the graphical user interface of claim 159 further configured to control, monitor, initiate, suspend or otherwise control delivery a chemotherapy protocol to a patient.

161. The computer controller or graphical user interface of any of claims 158, 159 or 160 adapted and configured for use with a device or implement a method as in claim 157.
